(19) United States
(12) Patent Application Publication
(10) Pub. No.: US 2023/0149562 A1
(43) Pub. Date: May 18, 2023

(54) CARRIERS FOR EFFICIENT NUCLEIC ACID DELIVERY

(71) Applicant: TIBA Biotech LLC, Cambridge, MA (US)

(72) Inventors: Poulami TALUKDER, Woburn, MA (US); Jasdave CHAHAL, Melrose, MA (US); Jin HUANG, Brighton, MA (US); Karl RUPING, Cambridge, MA (US)

(73) Assignee: TIBA Biotech LLC, Cambridge, MA (US)

(21) Appl. No.: 17/917,365

(22) PCT Filed: Apr. 2, 2021
(86) PCT No.: PCT/US2021/025542

§ 371 Date: Oct. 6, 2022

Related U.S. Application Data
(60) Provisional application No. 63/145,086, filed on Feb. 3, 2021.
(60) Provisional application No. 63/005,853, filed on Apr. 6, 2020.

(51) Int. Cl. A61K 47/69; C12N 15/88; A61K 47/54; A61K 47/59

(52) U.S. Cl. CPC A61K 47/6935; C12N 15/88; A61K 47/543; A61K 47/593

(57) ABSTRACT

Nanoparticle compositions for delivery of nucleic acids to subjects including carriers comprising polyester (PE) dendrimers or dendrons, and therapeutic or immunogenic nucleic acid agents enclosed within the PE are described. Methods for treating or preventing diseases or conditions in a subject by administering the nanoparticle compositions that provide immune responses and synergistic therapeutic or preventive effects are provided.

### CROSS REFERENCE TO RELATED APPLICATION

This application claims the benefit of U.S. provisional application No. 63/005,853, which was filed Apr. 6, 2020 and is titled CARRIERS FOR EFFICIENT NUCLEIC ACID DELIVERY, and of U.S. provisional application No. 63/145,086, which was filed Feb. 3, 2021 and is titled CARRIERS FOR EFFICIENT NUCLEIC ACID DELIVERY, both of which are incorporated herein by reference as if fully set forth.

### FIELD

The disclosure relates to carriers for efficient delivery of nucleic acids to a subject for treating or preventing diseases and/or disorders, and nanoparticle compositions comprising the carriers and nucleic acids. The disclosure also relates to methods of formulating the nanoparticle compositions and methods of treating diseases and/or disorders in the subjects with such nanoparticle compositions.

### BACKGROUND

In recent years, nucleic acid vaccines and therapeutics have emerged as a promising approach to prevent and treat several diseases or conditions, including applications in gene therapy. However, nucleic acids are large hydrophilic molecules which cannot penetrate through cell membranes and are susceptible to enzymatic degradation in the bloodstream. (Mendes et al., 2017, Molecules 22(9), 1401; Jones et al., 2013, Mol. Pharmaceutics 10, 4082-4098; and Nishikawa and Huang, 2001 Hum. Gene Ther. 12, 861-870).

Therefore, most of the proposed nucleic acid strategies depend on delivery vectors that, ideally, should overcome different extra- and intracellular barriers to efficiently deliver nucleic acids into cells with minimal toxicity (Jones et al., 2013, Mol. Pharmaceutics 10, 4082-4098; Gomes et al., 2014 MRS Bull. 39, 60-70; and Nishikawa and Huang, 2001 Hum. Gene Ther. 12, 861-870).

The obstacles toward a successful nucleic acid therapy include the following: nucleic acid degradation by endonucleases, cellular internalization, endosomal escape, payload release from the vector and access to the desired target, and vector intra- and extracellular accumulation. (Jones et al., 2013, Mol. Pharmaceutics 10, 4082-4098; Nishikawa and Huang, 2001 Hum. Gene Ther. 12, 861-870; Gomes et al., 2014 MRS Bull. 39, 60-70; and Dufes et al., 2005, Adv. Drug Delivery Rev. 57, 2177-2202).

Nonviral vectors, such as lipids, polymers, and dendrimers, have recently gained much attention (Jones et al., 2013, Mol. Pharmaceutics 10, 4082-4098; Nishikawa and Huang, 2001 Hum. Gene Ther. 12, 861-870; and Mintzer and Simanek, 2009, Chem. Rev. 109, 259-302). A common feature among these has been their cationic or ionizable nature. Among the non-viral vectors, dendrimer-based vectors have drawn great interest for over two decades as potential nucleic acid delivery vehicles. However accessing a biodegradable unimolecular carrier has remained a challenge (Mintzer and Grinstaff, 2010, Chem. Soc. Rev. 40, 173-190; Raviña et al., 2010, Macromolecules 43, 6953-6961; Nouri et al., 2012, J. Mater. Sci. Mater. Med. 23, 2967-2980; Pandita et al., 2011, Biomacromolecules 12, 472-481; Rodrigues et al., 2011, New J. Chem. 35, 1938-1943; Santos et al., 2010, J. Controlled Release 144, 55-64; Santos et al., 2009, J. Controlled Release 134, 141-148; Santos et al., 2010, Mol. Pharmaceutics 7, 763-774; and Duncan and Izzo, 2005, Adv. Drug Delivery Rev. 57, 2215-2237).

The ideal nucleic acid delivery vehicle should be biodegradable to prevent accumulation and subsequent cytotoxicity (Duncan and Izzo, 2005, Adv. Drug Delivery Rev. 57, 2215-2237). Polyester dendrimers called ‘biodendrimers’ have been reported, which have building blocks known to be biocompatible or degradable to natural metabolites in vivo (Carnahan and Grinstaff, 2001, J. Am. Chem. Soc. 123, 2905; Carnahan and Grinstaff, 2001, Macromolecules 34, 7648; and Carnahan and Grinstaff, 2006, Macromolecules 39, 609). While validated as vehicles for small molecule delivery, they are not suitable for nucleic acid delivery. Among other requirements, to be an efficient nucleic acid delivery carrier, a dendrimer should form complexes with the nucleic acid, preferably self-assembling into a nanoparticle composition that protects the nucleic acid from degradation while ensuring transport to the cell.

### SUMMARY

In an aspect, the invention relates to a nucleic acid carrier having the formula Ia or Ib:

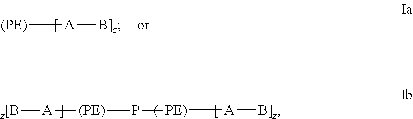

wherein PE is a polyester dendrimer or dendron which includes a core and a plurality of monomeric polyester units that form one or more generations, A is an amine linker, B is a hydrophobic unit, z is the number of surface groups, and P is the linker connecting two polyester dendrons.

In an aspect, the invention relates to a nanoparticle composition comprising any one of the nucleic acid carriers disclosed herein, a therapeutic or immunogenic nucleic acid agent enclosed therein, and a conjugated lipid.

In an aspect, the invention relates to a nanoparticle composition comprising any one of the nucleic acid carriers disclosed herein, a therapeutic or immunogenic nucleic acid agent enclosed therein, one conjugated lipid (e.g PEG-Lipid) disclosed herein, and a mixture of a phospholipid and cholesterol or a derivative thereof to improve with intracellular delivery as well as nanoparticle stability in vivo.

In an aspect, the invention relates to a method for treating or preventing a disease or condition in a subject. The method involves providing any one of the nanoparticle compositions disclosed herein and administering a therapeutically effective amount of these nanoparticle compositions to a subject.

### BRIEF DESCRIPTION OF THE DRAWINGS

The following detailed description of preferred embodiments of the present invention will be better understood when read in conjunction with the appended drawings. For the purpose of illustrating the invention, particular embodiments are shown in the drawings. It is understood, however, that the invention is not limited to the precise arrangements and instrumentalities shown. In the drawings:

FIG. 1 is a schematic drawing of generation 1 modified polyester dendrimer with fatty acid side chains (B) that were used for modification. In this figure, the fatty acid side chain B can be selected from any one of C₄-C₂₈ fatty acids.

FIG. 2 illustrates a process for preparing a nanoparticle composition designed for improved self-assembly which includes a modified dendrimer (PE-stearic), 1,2 dimyristoyl-sn-glycero-3-phosphoethanolamine-N-[methoxy(polyethylene glycol)-2000], and mRNA.

FIG. 3 illustrates the distribution of the nanoparticle compositions measured as the intensity based on size (d) of the nanoparticles.

FIG. 4 illustrates a photograph of the agarose gel showing the binding of the modified dendrimer with RNA. The gels were stained with ethidium bromide (EB) and gel images were taken on a Syngene G Box imaging system (Syngene, USA).

FIG. 5 shows the stability of dendrimers at neutral pH and pH 5.0 that was used in the process of formulating RNA.

FIGS. 6A-6E show the stability of RNA-PE stearic nanoparticles in PBS as measured by DLS and by agarose gel retention assay:

FIG. 6A illustrates the distribution of the nanoparticle compositions (right after dialysis) measured as the intensity based on size (d) of the nanoparticles;

FIG. 6B shows the stability of PE-Stearic PR8 HA mRNA as measured by DLS following storage for 3 weeks at 4° C.;

FIG. 6C shows the stability of PE-Stearic PR8 HA mRNA as measured by DLS following storage for 3 days at room temperature (RT);

FIG. 6D shows the stability of PE-Stearic PR8 HA mRNA as measured by DLS following storage for 2 hours at 37° C.; and

FIG. 6E shows the stability results based on the gel retention assay: lane 1—PR8 HA mRNA, lane 2—PE-Stearic PR8 HA mRNA, 4 deg, 3 weeks, lane 3—PE-Stearic PR8 HA mRNA, rt, 3 days, lane 4—PE-Stearic PR8 HA mRNA, 37° C., 1 hour, and lane 5—PE-Stearic PR8 HA mRNA, 37° C., 2 hours.

FIGS. 7A and 7B show luciferase expression in cell culture from luciferase mRNA delivered into mammalian cells by modified polyester dendrimer-based nanoparticles and measured by quantification of intracellular luciferase activity using a luminescence assay:

FIG. 7A illustrates cell culture expression of luciferase mRNA delivered by nanoparticles containing PE-linoleic, PE-stearic, PE-palmitic compared to the naked luciferase mRNA (negative control); and

FIG. 7B illustrates cell culture expression of luciferase mRNA delivered by nanoparticles containing PE-heptadecanoic, PE-stearic, PE-oleic, and PE-16-hydroxypalmitic compared to the naked luciferase mRNA.

FIG. 8 illustrates the Western blot analysis of HA expression in cell culture following delivery of PR8 HA mRNA in modified polyester dendrimer-based nanoparticles.

FIG. 9 illustrates HA specific antibody induced by intramuscular injection of PR8 HA mRNA-containing nanoparticles and assayed by Hemagglutinin Inhibition Assay (HAI).

FIG. 10 illustrates distribution of the DNA nanoparticle compositions measured as the intensity based on size (d) of the nanoparticles.

FIG. 11 shows in vitro SEAP expression via quantiblue assay for nanoparticles containing DNA and modified polyester dendrimer.

FIG. 12 illustrates the SEAP colorimetric signal for the replicon RNA expressing SEAP that was formulated into modified polyester dendron nanoparticles at a pH of 4.0 or 5.0.

FIG. 13 illustrates Western Blot analysis of Spike expression in cell culture following delivery of SARS-CoV-2 Spike Replicon RNA in polyester dendrimer and dendron based nanoparticles.

FIG. 14 illustrates endpoint dilution serum titer for mouse IgG specific to COVID-19 Spike protein in response to vaccination with Replicon Spike RNA formulated with PE dendron-G2-ricinoleic delivery material.

FIG. 15 illustrates the relative Luciferase expression, measured in relative light units (RLU) in heart and spleen at 6, 16, and 42 hours post-treatment from mice injected with 7.2 μg of Luciferase-encoding replicon RNA formulated with PE dendron G2-5A2-5 ricinoleic or 31.4 μg of the same RNA formulated as lipid nanoparticles (LNP).

FIG. 16 illustrates distribution of the nanoparticle compositions measured as the intensity based on size (d) of the nanoparticles.

FIG. 17 illustrates the SEAP colorimetric signal for the replicon RNA expressing SEAP that was formulated into, PE Dendron_G2-A1-Ricinoleic and (PE Dendron_G2-A1-Ricinoleic)2 PEG 200 nanoparticles.

### DETAILED DESCRIPTION

Certain terminology is used in the following description for convenience only and is not limiting.

The “nanoparticle composition” refers to a composition that includes a modified dendrimer and a nucleic acid payload molecule enclosed therein.

The term “substitute” refers to the ability to change one functional group, or a moiety included therein, for another functional group or moiety therein, provided that the valency of all atoms on the parent structure is maintained. The substituted group is interchangeably referred herein as “substitution” or “substituent.” When more than one position in any given structure is substituted with more than one substituent selected from a specified group, the substituent may be either the same or different at every position.

The term “amine linker” as used herein, refers to an amine containing linker that links or connects the hydrophobic tails (described as component “B” herein for convenience) with the terminal chemical groups present on the dendrimer or dendron surface. Amines as present in the amine linker are functional groups that contain a basic nitrogen atom with a lone pair. Amines are formally derivatives of ammonia, wherein one or more hydrogen atoms have been replaced by a substituent such as an alkyl group.

The term “alkyl as used herein, means a straight or branched chain hydrocarbon containing from 1 to 28, preferably 1 to 20, carbon atoms unless otherwise specified. Alkyl chain length may be used to control hydrophobicity and self-assembly properties of nucleic acid carrier. Representative examples of alkyl include, but are not limited to, methyl, ethyl, n-propyl. iso-propyl. n-butyl, sec-butyl, iso-butyl, tert-butyl, n-pentyl, isopentyl, neopentyl, n-hexyl, 3-methylhexyl, 2,2-dimethyl pentyl, 2,3-dimethylpentyl, n-heptyl, n-octyl, n-nonyl, and n-decyl. When an “alkyl group is a linking group between two other moieties, then it may also be a straight or branched chain. Examples of an alkyl group include, but are not limited to —CH2-, —CH2CH2-, —CHCHCHC(CH), and CHCH(CHCH)CH—.

The term “surface groups” as used herein, means terminal groups on the surface of nucleic acid carriers. The surface of the nucleic acid carriers herein are modified with hydrophobic tails (described as component “B” herein) in order to assist self-assembly properties.

The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C” or “A, B, and C” means any individual one of A, B or C as well as any combination thereof.

In an embodiment, a nucleic acid carrier having the formula Ia or Ibis provided:

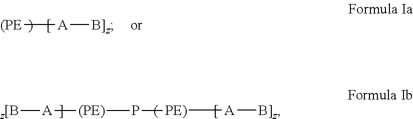

wherein PE is a polyester dendrimer or dendron, which includes a core and monomeric polyester units layered around the core to form a tree-like structure, where each layer is called a generation (G), A is an amine linker, B is a hydrophobic unit, z is the number of surface groups, and P is the linker connecting two polyester dendrons. The amine linker may contain protonated, charged, amine groups at physiologic pH.

In an embodiment, PE may have Formula II:

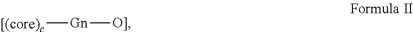

wherein c is the core multiplicity or number of wedges originating from the core, c values range from 1 to 6. The dendron may consist of a hyperbranched wedge emanating from a single chemically addressable focal point, also referred to herein as a core. Thus, for dendrons with unidirectional core, c equals 1. G is a layer or generation of dendrimer or dendron; n is a generation number, whose values range from 1 to 10; and the monomeric polyester unit may be 2,2-bis(hydroxymethyl) propionic acid or 2,2-Bis(hydroxymethyl)butyric acid; z has Formula III:

z=cbⁿ, Formula III

wherein bis branch point multiplicity, or number of branches at each branching point; c ranges from 1 to 6, and n is a generation number.

In the nucleic acid carrier that comprises 2,2-bis(hydroxymethyl) propionic acid or 2,2-Bis(hydroxymethyl)butyric acid as the monomeric polyester unit, branch point multiplicity or number of branches at each branching point, bis 2.

The structure of the core may influence the number of functional groups on the surface, amine and/or charge density, diameter, and flexibility of the resultant nucleic acid carrier which may modulate the carrier's physicochemical properties, interaction with nucleic acid, and gene transfer activity.

In an embodiment, the core may be a unidirectional, wherein c is 1 in Formula II. The unidirectional core may be a carboxylic acid or derivative thereof.

The unidirectional cores may be selected from the following scaffolds:

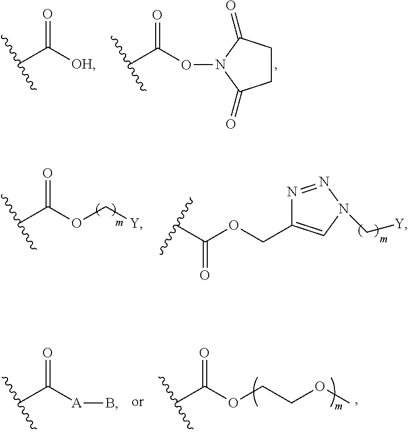

wherein Y is selected from methyl, iso-propyl, sec-butyl, iso-butyl, tert-butyl, isopentyl, neopentyl, 3-methylhexyl, 2,2-dimethylpentyl, 2,3-dimethylpentyl, azide (N3), halogen (Cl, Br, or I), acetylene (C₂H₂), hydroxyl (—OH), or thiol (—SH), pyranosyl, cycloalkyl, aryl, heteroaryl, and heterocycle, wherein each of the cycloalkyl, aryl, heteroaryl, and heterocycle may be substituted with halogen, hydroxyl (—OH) and alkyl group; A is an amine linker; B is a hydrophobic unit; and m is 1 to 20.

In an embodiment, the core may be a three directional core, wherein c is 3 in Formula II. The three directional core may be trimethylol propane, or 1,1, 1-tris(hydroxyphenylethane). For reference, the structures of the above cores are presented pictorially as the following scaffolds:

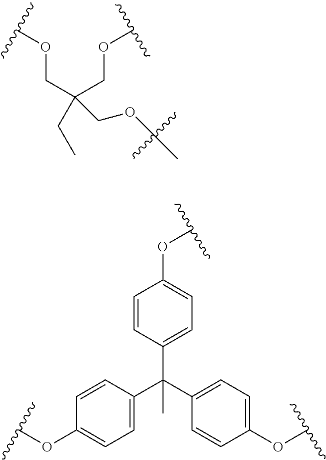 [SMILES: *OCC(CC)(CO*)CO* ; *Oc1ccc(C(C)(c2ccc(O*)cc2)c2ccc(O*)cc2)cc1]

In an embodiment, the core may be a four directional core, wherein c is 4 in Formula II.

The four directional core may be but is not limited to pentaerythritol, adamantane-1,3,5,7-tetraol, or 5,10,15,20-Tetrakis(4-hydroxyphenyl)-21H,23H-porphine, [1,1′-biphenyl]-3,3′,5,5′-tetraol, 2,3,6,7-tetrahydroxy-9,10-dimethyl-anthracene, 9,10-dimethyl-9,10-dihydro-9,10-ethanoanthracene-2,3,6,7-tetraol, 6,13-dihydro-pentacene-5,7,12,14-tetraol, Hexahydro-[1,4]dioxino[2,3-b][1,4]dioxine-2,3,6,7-tetraol, Anthracene-1,4,9,10-tetraol, pyrene-1,3,6,8-tetraol, or 3,3,3′,3′-tetramethyl-2,2′,3,3′-tetrahydro-1,1′-spirobi[indene]-5,5′,6,6′-tetrol. These four directional cores are illustrated as the following scaffolds:

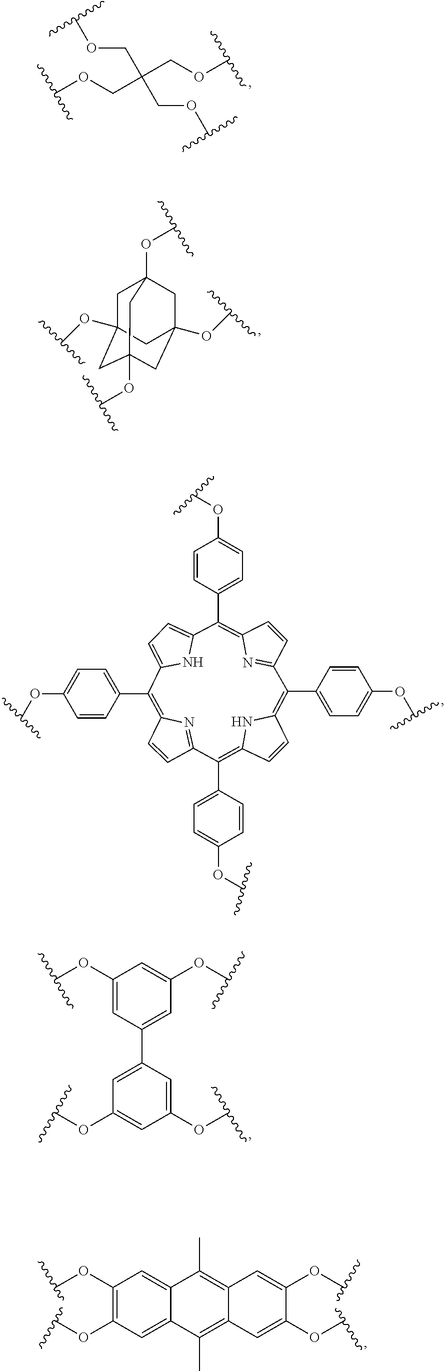 [SMILES: *OCC(CO*)(CO*)CO* ; *OC12CC3(O*)CC(O*)(C1)CC(O*)(C2)C3 ; *Oc1ccc(-c2c3nc(c(-c4ccc(O*)cc4)c4ccc([nH]4)c(-c4ccc(O*)cc4)c4nc(c(-c5ccc(O*)cc5)c5ccc2[nH]5)C=C4)C=C3)cc1 ; *Oc1cc(O*)cc(-c2cc(O*)cc(O*)c2)c1 ; *Oc1cc2c(C)c3cc(O*)c(O*)cc3c(C)c2cc1O*]

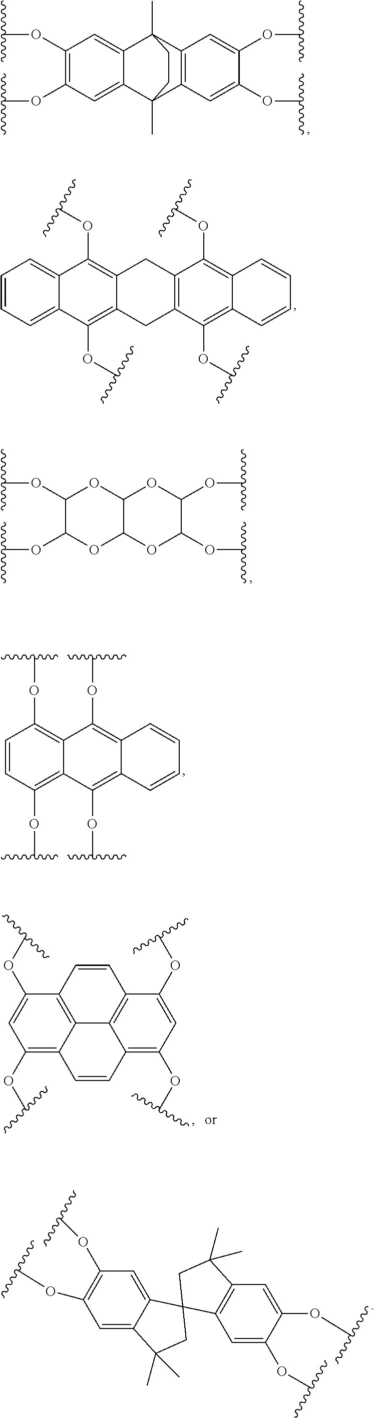 [SMILES: *Oc1cc2c(cc1O*)C1(C)CCC2(C)c2cc(O*)c(O*)cc21 ; *Oc1c2c(c(O*)c3ccccc13)Cc1c(c(O*)c3ccccc3c1O*)C2 ; *OC1OC2OC(O*)C(O*)OC2OC1O* ; *Oc1ccc(O*)c2c(O*)c3ccccc3c(O*)c12 ; *Oc1cc(O*)c2ccc3c(O*)cc(O*)c4ccc1c2c43 ; *Oc1cc2c(cc1O*)C1(CC2(C)C)CC(C)(C)c2cc(O*)c(O*)cc21]

The amine linker A is a moiety that imparts proton-accepting functionality to the nucleic acid carrier molecule by containing one or more nitrogen atoms with lone pairs. The amine linker is thus able to accept a free proton (H+) under acidic conditions. In preferred embodiments the nitrogen atom(s) are present in the form of secondary or tertiary amines. The amine linker may be derived from N1-(2-aminoethyl)ethane-1,2-diamine, N1-(2-aminoethyl)propane-1,3-diamine, N1-(3-aminopropyl)propane-1,3-diamine, N1,N1′-(ethane-1,2-diyl)bis(ethane-1,2-diamine), N1,N1′-(ethane-1,2-diyl)bis(N2-(2-aminoethyl)ethane-1,2-diamine), N1-(2-(4-(2-aminoethyl)piperazin-1-yl)ethyl)ethane-1,2-diamine, N1-(2-aminoethyl)-N1-methylethane-1,2-diamine, N1-(3-aminopropyl)-N1-methylpropane-1,3-diamine, N1-(3-aminopropyl)-N1-ethylpropane-1,3-diamine, 3-((3-aminopropyl)(methyl)amino)propan-1-ol, 3,3′-(methylazanediyl)bis(propan-1-ol), N1-(3-aminopropyl)-N1-methylbutane-1,4-diamine, 4-((3-aminopropyl)(methyl)amino)butan-1-ol, 4-((3-hydroxypropyl)(methyl)amino)butan-1-ol, 4-((3-hydroxypropyl)(methyl)amino)butan-1-ol, N1-(4-aminobutyl)-N1-methylbutane-1,4-diamine, 4-((4-aminobutyl)(methyl)amino)butan-1-ol, 4,4′-(methylazanediyl)bis(butan-1-ol), 3-((3-aminopropyl)(ethyl)amino)propan-1-ol, 3,3′-(ethylazanediyl)bis(propan-1-ol), N1-(3-aminopropyl)-N1-ethylbutane-1,4-diamine, 4-((3-aminopropyl)(ethyl)amino)butan-1-ol, 4-(ethyl(3-hydroxypropyl)amino)butan-1-ol, N1-(2-aminoethyl)-N1-methylpropane-1,3-diamine, N1-(4-aminobutyl)-N1-ethylbutane-1,4-diamine, 4,4′-(ethylazanediyl)bis(butan-1-ol), 3-((3-aminopropyl)amino)propan-1-ol, N1-(3-aminopropyl)butane-1,4-diamine, 4-((3-hydroxypropyl)amino)butan-1-ol, N1-(4-aminobutyl)butane-1,4-diamine, 3,3′-azanediylbis(propan-1-ol), 4-((3-aminopropyl)amino)butan-1-ol, 4,4′-azanediylbis(butan-1-ol), or N1,N1′-(butane-1,4-diyl)bis(propane-1,3-diamine). For reference, the structures of the above amines are presented pictorially in the following structures:

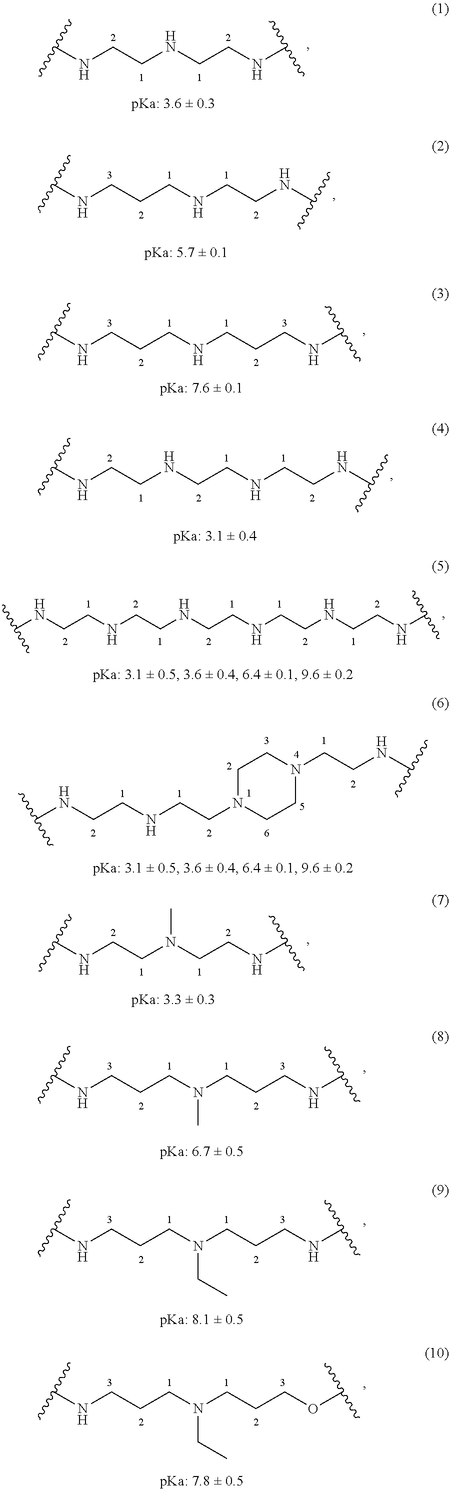

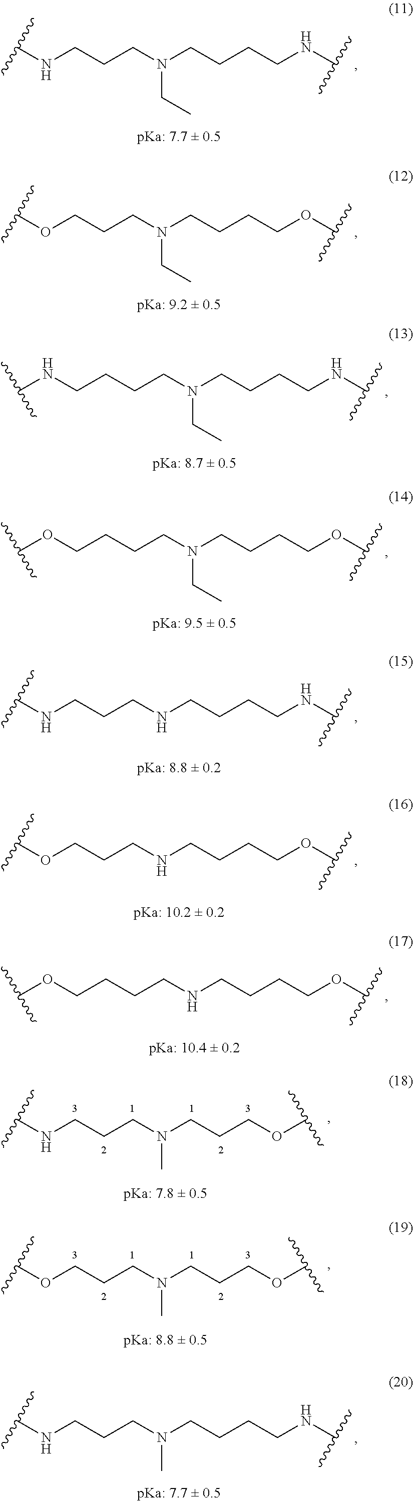

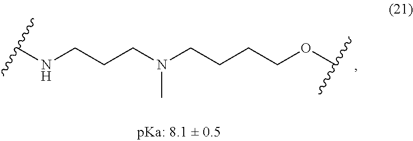 [SMILES: *NCCCN(C)CCCCO*]

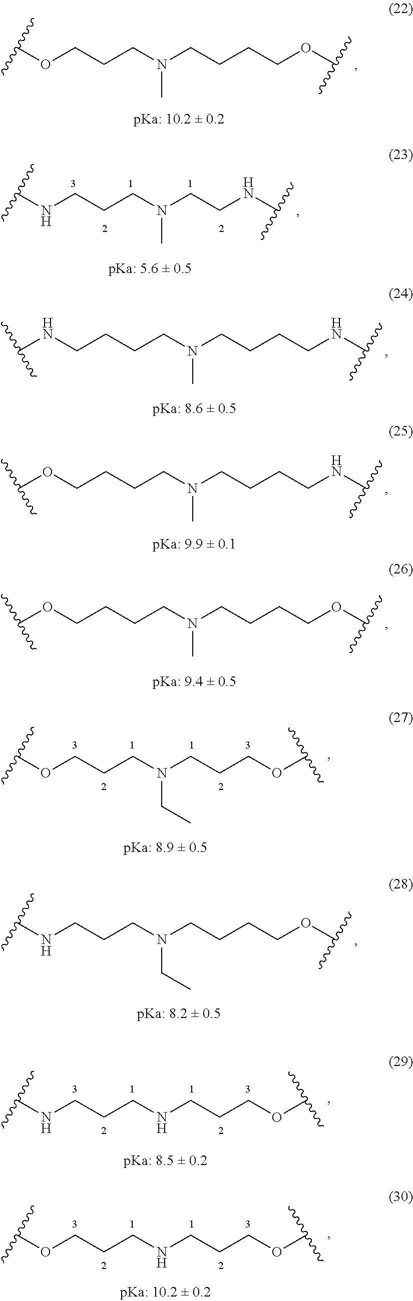

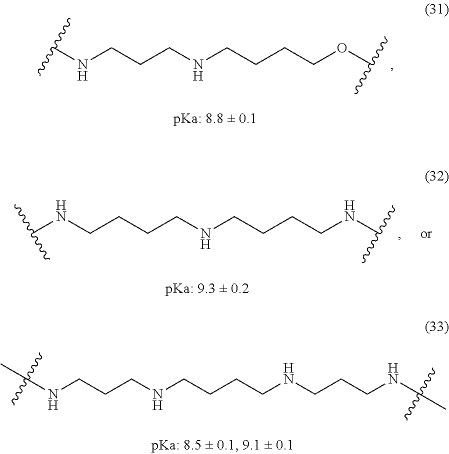 [SMILES: *NCCCNCCCCO* ; *NCCCCNCCCCN* ; *NCCCNCCCCNCCCN*]

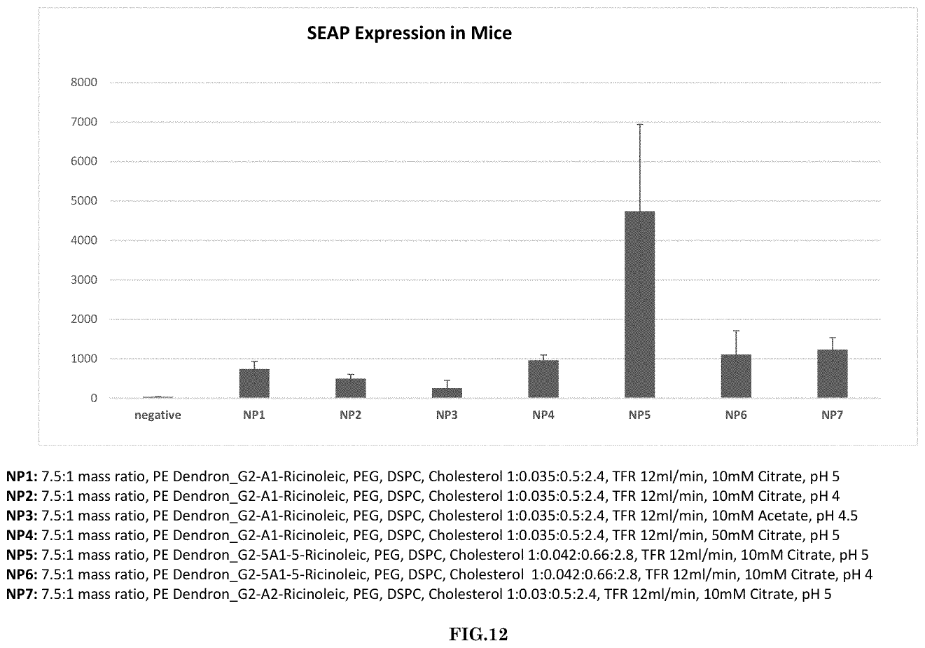

As listed above, protonatable dendrimers/dendrons may have an acid dissociation constant (pKa) of the protonatable group in the range of about 3.3 to about 10.4. These delivery molecules will be cationic during formulation with nucleic acids under acidic pH conditions. Under such conditions, ionic interaction will cause them to condense with the negatively charged nucleic acids. While analyzing the structure-activity relationship of ionizable polyester dendrimer/dendron molecules an unexpected relationship was discovered between the pKa of the ionizable delivery molecules with the ability of the nanoparticles to deliver functionally active Replicon RNA. As an example, delivery molecules containing Amine1 and Amine2 illustrated below with predicted pKa values of 6.7 and 7.7 respectively were able to deliver an mRNA to cells (as evidenced by SEAP expression in FIG. 12) whereas a dendrimer molecule containing Amine 3 (predicted pKa 3.3) was not able to deliver the payload. The pKa values were calculated using ACD/percepta pKa prediction tool.

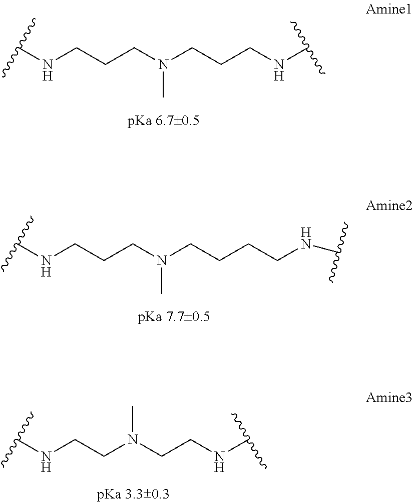

The hydrophobic unit B may be a C₁-C₂₂ alkyl or C₂-C₂₂ alkenyl group. Each of the C₁-C₂₂ alkyl or C₂-C₂₂ alkenyl. group may be optionally substituted with one to four substituents selected from halogen, —CN, —NO₂, —N₃, C₁-C₆ alkyl, halo(C₁-C₆ alkyl), —OR, —NR₂, —CO₂R, —OC(O)R, —CON(R)₂, —OC(O)N(R)₂, —NHC(O)N(R)₂, —NHC(NH)N(R)₂, C₃-C₈ cycloalkyl, C₃-C₈ cycloalkenyl, aryl, heteroaryl, or heterocycle. Each R may independently be selected from hydrogen, C₁-C₆ alkyl, halo(C₁-C₆ alkyl), C₃-C₈ cycloalkyl, C₃-C₈ cycloalkenyl, aryl, heteroaryl, or heterocycle. Each cycloalkyl, cycloalkenyl, aryl, heteroaryl, and heterocycle may be further optionally substituted with R′, wherein R′ may independently be selected from halogen, —CN, —NO₂, —N₃, C₁-C₆ alkyl, or halo(C₁-C₆ alkyl).

The hydrophobic unit B of Formula Ia and Formula Ib may be introduced by contacting the PE dendrimer or dendron with a functional reagent such as fatty acid or its derivatives. The fatty acid may be saturated or unsaturated fatty acid having C4-C28 chains. The fatty acid may be, but is not limited to, arachidonic acid, oleic acid, eicosapentaenoic acid, lauric acid, caprylic acid, capric acid, myristic acid, palmitic acid, stearic acid, linoleic acid, or linolenic acid. The fatty acid derivative may be, but is not limited to, 12-hydroxy-9-cis-octadecenoic acid, 12-methyltetradecanoic acid, 12-methyltridecanoic acid, 14-methylhexadecanoic acid, 14-methylhexadecanoic acid, 18-methylnonadecanoic acid, 19-methylarachidic acid, isopalmitic acid, isostearic acid, phytanic acid, (±)-2-hydroxyoctanoic acid, (±)-3-hydroxydecanoic acid, (±)-3-hydroxyoctanoic acid, 10-hydroxydecanoic acid, 12-hydroxyoctadecanoic acid, 15-hydroxypentadecanoic acid, 16-hydroxyhexadecanoic acid, 2-hydroxyhexadecanoic acid, 2-hydroxytetradecanoic acid, 2-hydroxydodecanoic acid, DL-α-hydroxystearic acid, DL-β-hydroxylauric acid, DL-β-hydroxymyristic acid, or DL-β-hydroxypalmitic acid.

The hydrophobic unit B may be a methyl, ethyl, propyl, butyl, pentyl, hexyl, heptyl, octyl, decyl, undecyl, dodecyl, tridecyl, tetradecyl, pentadecyl, hexadecyl, heptadecyl, octadecyl, but-3-en-1-yl, oct-7-en-1-yl, 12-tridecenyl, 14-pentadecenyl, 17-octadecenyl, oleyl, linoleyl, arachidoneyl, or 16-hydroxyhexadecyl group.

The hydrophobic unit B of the nucleic acid carrier of Formula I may be an unsaturated alkyl group. The presence of unsaturated alkyl groups in the nucleic acid carrier may prevent nanoparticle recycling when the carrier is formulated into nanoparticle composition. The unsaturated alkyl groups may be more fluid, and may have a lower crystallization temperature compared to the saturated alkyl groups, and thus, may have the ability to morphologically change nanoparticles containing the nucleic acid carrier into a fusogenic form when they interact with phospholipid bilayers of the cell membrane and to rupture the endosome. Thus, the nanoparticles may become restricted and may remain inside the cells at the site of injection only and may not be trafficked elsewhere.

In an embodiment, the nucleic acid carrier may comprise functional groups suitable for tracking the delivery material in vitro and in vivo. The nucleic acid carrier may have the fatty acids containing stable isotopes of carbon (C) or hydrogen (H), such as ¹³C or ²H (also referred to herein as deuterium, D or d). When the nucleic acid carrier is formulated into nanoparticles with nucleic acids, such as replicon RNA, the nanoparticles may be tracked in vitro and in vivo post-administration by techniques such as mass spectroscopy or nuclear magnetic resonance imaging. The inclusion of the stable isotopes may be beneficial for identification of the delivery molecules since these isotopes differ from the abundant in tissues ¹²C and ¹H isotopes. Tracking may be useful for identifying biodistribution, material clearance and molecular stability of nanoparticles post-administration, and related issues. The isotopically labeled fatty acids may be, but are not limited to, octanoic acid-1-¹³C, octanoic acid ¹³C, octanoic acid-8,8,8-²H₃, octanoic-²H15 acid, decanoic acid-1-¹³C, decanoic acid-10-¹³C, decanoic-10,10,10-²H3 acid, decanoic-²H19 acid, undecanoic acid ¹³C, lauric acid-12,12,12-²H₃, lauric-²H23 acid, lauric acid-1-¹³C, lauric acid-1,12-¹³C2, tridecanoic-2,2-²H2 acid, myristic acid-14-¹³C, myristic acid-1-¹³C, myristic acid-14,14,14-²H₃, myristic-d27 acid, palmitic acid-1-¹³C, palmitic acid-16-¹³C, palmitic acid-16-¹³C,16,16,16-²H₃, palmitic acid-²H31, stearic acid-1-¹³C, stearic acid-18-¹³C, stearic acid-18,18,18-²H₃, stearic-²H35 acid, oleic acid-1-¹³C, oleic acid-²H34, linolenic acid-1-¹³C, linoleic acid-²H32, arachidonic-5,6,8,9,11,12,14,15-²H8 acid, or eicosanoic-²H39 acid.

The linker unit P of Formula Ib may be a homobifunctional linker with two azide groups. It can be used for the synthesis of dimeric molecules. In an embodiment, P may have Formula IV:

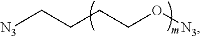

where m ranges from 1 to 20.

In an embodiment, a nanoparticle composition comprising any one of the nucleic acid carriers described herein is provided. A nanoparticle composition herein may be useful to introduce an agent into a cell. The agent may be a nucleic acid. A nanoparticle composition herein may be useful as a transfection agent. A nanoparticle composition herein may be useful in a method of treating.

In an embodiment, a nanoparticle composition may comprise a mixture of nucleic acid carriers, each one of them comprising different amine density or side chains. These nucleic acid carriers may be mixed at a fixed ratio. For an example of mixture with three dendrimers, a ratio of the first nucleic acid carrier to the second nucleic acid carrier and to the third nucleic acid carrier may be i:j:k where i, j and k

are independently selected from 1, 2, 3, 4, 5, 6, 7, 8, 9, 10, 11, 12, 13, 14, 15, 16, 17, 18, 19, or 20, or a value between any two of the foregoing.

In an embodiment, the nanoparticle composition may comprise one or more therapeutic or immunogenic nucleic acid agents. As used herein, the term “nucleic acid” refers to any natural or synthetic DNA or RNA molecules. A therapeutic or immunogenic nucleic acid agent of a composition herein may be complexed with or encapsulated in a nucleic acid carrier of the nanoparticle composition.

In an embodiment, the therapeutic or immunogenic nucleic acid agent may be an RNA or DNA molecule. The term “DNA” or “DNA molecule” or “deoxyribonucleic acid molecule” refers to a polymer of deoxyribonucleotides. The DNA molecule may be a polynucleotide, oligonucleotide, DNA, or cDNA. The DNA molecule may encode wild-type or engineered proteins, peptides or polypeptides, such as antigens. The term “RNA” or “RNA molecule” or “ribonucleic acid molecule” refers to a polymer of ribonucleotides (e.g., 2, 3, 4, 5, 10, 15, 20, 25, 30, or more ribonucleotides). The RNA molecule may be a replicon RNA (repRNA), small interfering RNA (siRNA), miRNA, single strand guide RNA (sgRNA), messenger RNA (mRNA), or transfer RNA (tRNA). Replicon RNA (repRNA) refers to a replication-competent, progeny-defective RNA virus genome that is incapable of producing infectious progeny virions. Viral genomes that are typically modified for use as repRNAs include “positive strand” RNA viruses. The modified viral genomes function as both mRNA and templates for replication. Small interfering RNA (siRNA) refers to an RNA (or RNA analog) comprising between about 10-50 nucleotides (or nucleotide analogs) which is capable of directing or mediating RNA interference. MicroRNAs (miRNAs) refers to small (20-24 nt) regulatory non-coding RNAs that are involved in post-transcriptional regulation of gene expression in eukaryotes by affecting either or both the stability and translation of coding mRNAs. Messenger RNAs (mRNAs) are usually single-stranded RNAs and define the amino acid sequence of one or more polypeptide chains. This information is translated during protein synthesis when ribosomes bind to the mRNA. The DNA or RNA molecules may be chemically modified.

The RNA molecule may be a monocistronic or polycistronic mRNA. A monocistronic mRNA refers to an mRNA comprising only one sequence encoding a protein, polypeptide or peptide. A polycistronic mRNA typically refers to two or more sequences encoding two or more proteins, polypeptides or peptides. An mRNA may encode a protein, polypeptide, or peptide that acts as an antigen.

In an embodiment, the DNA molecule may be a polynucleotide, oligonucleotide, DNA, or cDNA. The RNA molecule may be a replicon RNA (repRNA), small interfering RNA (siRNA), miRNA, single strand guide RNA (sgRNA), messenger RNA (mRNA), or transfer RNA (tRNA). The therapeutic or immunogenic nucleic acid agent may be non-covalently bound or covalently bound to the nucleic acid carrier. The therapeutic or immunogenic nucleic acid agent may be electrostatically bound to the charged nucleic acid carrier through an ionic bond.

In an embodiment, the nanoparticle compositions described herein may include immunogenic or therapeutic nucleic acid agents encoding antigens.

As used herein, “encapsulated” can refer to a nanoparticle that provides an active agent or therapeutic agent, such as a nucleic acid (e.g., a messenger RNA), with full encapsulation, partial encapsulation, or both. In a preferred embodiment, the nucleic acid is fully encapsulated in the nanoparticle. In the context of nucleic acid therapeutic agents, full encapsulation may be determined by a Ribogreen® assay. RiboGreen® is an ultra-sensitive fluorescent nucleic acid stain for quantitating oligonucleotides and single-stranded DNA or RNA in solution (available from Thermo Fisher Scientific—US).

“Antigen” as used herein is defined as a molecule that triggers an immune response. The immune response may involve either antibody production, or the activation of specific immunologically active cells, or both. The antigen may refer to any molecule capable of stimulating an immune response, including macromolecules such as proteins, peptides, or polypeptides. The antigen may be a structural component of a pathogen, or a cancer cell. The antigen may be synthesized, produced recombinantly in a host, or may be derived from a biological sample, including but not limited to a tissue sample, cell, or a biological fluid.

The antigen may be but is not limited to a vaccine antigen, parasite antigen, bacterial antigen, tumor antigen, environmental antigen, therapeutic antigen or an allergen. As used herein a nucleotide vaccine is a DNA- or RNA-based prophylactic or therapeutic composition capable of stimulating an adaptive immune response in the body of a subject by delivering antigen(s). The immune response induced by vaccination typically results in development of immunological memory, and the ability of the organism to quickly respond to subsequent encounter with the antigen or infectious agent.

The use of a “nucleic acid carrier” herein as a carrier of nucleic acids is preferred and the name “nucleic acid carrier” is applied for that reason. However, embodiments also include the combination of a nucleic acid carrier herein with an agent that contains negative or partially negative charges. The agent may be a drug, a protein, or a lipid conjugate.

In an embodiment, the nanoparticle composition may be formulated to include drugs that contain negative or partially negative charges. The nanoparticles may be formulated via the electrostatic association of the negative charge with the positive charge of protonated amine groups in the nucleic acid carrier. The negatively charged drugs may be ionic drugs. The term “ionic drug” refers to an electrically asymmetric molecule, which is water soluble and ionizable in solution of distilled water. The ionic drugs may contain phosphate, phosphonate, or phosphinate functional groups. The drugs including phosphate groups may be phosphate-containing nucleotide analogs, for example, drugs used for treating cancer and viral chemotherapy. The phosphate-containing drugs may be, but are not limited to, purine and pyrimidine nucleoside analogs, Arabinosylcytosine (ara-C), Ara-C monophosphate (ara-CMP), azidothymidine (AZT), AZT monophosphate (AZTMP), 2′3′-dideoxycytidine (ddCD), cyclic adenoside monophosphate (cAMP), tenofovir, or adefovir.

In an embodiment, the nanoparticle composition may comprise one or more proteins. Non-limiting examples of the one or more proteins include antibodies or antibody fragments, cytokines such as interferon (IFN)-alpha or interleukin (IL)-2, pathogen-derived antigens such as SARS-CoV Spike protein or the receptor-binding domain (RED) thereof, cancer-derived antigens such as mutant forms of Kirsten rat sarcoma 2 viral oncogene homolog (KRAS), or other therapeutic biologics such as insulin, factor VIII, or erythropoietin. A protein of a composition herein may be in the bulk composition, and/or complexed with or encapsulated in a nucleic acid carrier of the nanoparticle composition.

In an embodiment, the nanoparticle composition described herein may comprise a lipid conjugate. The lipid conjugate may be useful in that it may prevent the aggregation of particles. Lipid conjugates that may be in a composition herein include, but are not limited to, PEG-lipid conjugates. Non-limiting examples of PEG-lipids include, PEG coupled to lipids such as DMG-PEG 2000, PEG coupled to phospholipids such as phosphatidylethanolamine (PEG-PE), PEG conjugated to cholesterol or a derivative thereof, and mixtures thereof. In certain instances, the PEG may be optionally substituted by an alkyl, alkoxy, acyl, or aryl group.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Avanti Polar Lipids. The PEG moiety of the PEG-lipid conjugates described herein may comprise an average molecular weight ranging from about 550 daltons to about 10,000 daltons.

Phosphatidylethanolamines having a variety of acyl chain groups of varying chain lengths and degrees of saturation can be conjugated to PEG to form the lipid conjugate. Phosphatidylethanolamines are commercially available, or can be isolated or synthesized using conventional techniques. The phosphatidylethanolamines may comprise saturated or unsaturated fatty acids with carbon chain lengths in the range of C₁₀ to C₂₀. The phosphatidylethanolamines may comprise mono- or polyunsaturated fatty acids and mixtures of saturated and unsaturated fatty acids. The phosphatidylethanolamines contemplated include, but are not limited to, dimyristoylphosphatidylethanolamine (DMPE), dipalmitoyl-phosphatidylethanol amine (DPPE), dioleoylphosphatidylethanolamine (DOPE), and distearoyl-phosphatidylethanolamine (DSPE).

The PEG-lipid may comprise PEG conjugated to cholesterol or cholesterol derivative. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof.

The size, relative quantity and distribution of the PEG-lipid, included in the nanoparticle composition may affect physical properties of the nanoparticle composition. The physical properties that can be controlled may be, but are not limited to, diameter of the nanoparticle, the propensity of the nanoparticles to aggregate, the number of nucleic acid molecules inside each nanoparticle, or the concentration of the nanoparticles in the nanoparticle composition, the efficacy of the intra-cellular delivery of therapeutic and immunogenic nucleic acid agents, and/or the efficacy of uptake of the nanoparticles by cells.

The nanoparticle composition may contain 10 mol % or less of the PEG-lipid per nanoparticle composition. The nanoparticle composition may comprise about 10 mol %, about 9 mol %, about 8 mol %, about 7 mol %, about 6 mol %, about 5 mol %, about 4 mol %, about 3 mol %, about 2 mol %, or about 1 mol %, or any amount in between any two of the foregoing integers of the PEG-lipid per nanoparticle composition. The nanoparticle composition comprising the PEG-lipid may comprise nanoparticles with a smaller diameter than nanoparticles of the composition lacking the PEG-lipid. The nanoparticle composition may also comprise nanoparticles having a higher propensity of the nanoparticles to aggregate than nanoparticles of the composition lacking the PEG-lipid.

The nanoparticle composition may contain “amphipathic lipid.” As used herein, “amphipathic lipid” refers to any material having non-polar hydrophobic “tails” and polar “heads.” Polar groups may include phosphate, carboxylic, sulfato, amino, sulfhydryl, nitro, hydroxyl, or other like groups. Nonpolar groups may include, but are not limited to, long-chain saturated and unsaturated aliphatic hydrocarbon groups and such groups substituted by one or more cycloalkyl, cycloalkenyl, aryl, heteroaryl, or heterocycle group(s). Examples of amphipathic lipids include, but are not limited to, phospholipids, aminolipids, and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidyl choline, dioleoylphos-phatidylcholine, distearoylphosphatidylcholine, and dilinoleoylphosphatidylcholine.

The nanoparticle composition may contain the amphipathic lipid in the amount ranging from 10 mol % to 15 mol % of the amphipathic lipid per nanoparticle composition.

In an embodiment, the nanoparticle composition may include cholesterol or cholesterol derivative. Examples of cholesterol derivatives include, but are not limited to, cholestanol, 5,6-epoxy cholestanol, cholestanone, cholestenone, coprostanol, cholesteryl-2′-hydroxyethyl ether, cholesteryl-4′-hydroxybutyl ether, 24-ethyl cholesterol, 24-methyl cholesterol, cholenic Acid, 3-hydroxy-5-cholestenoic Acid, cholesteryl palmitate, cholesteryl arachidonate, cholesteryl arachidate, cholesteryl myristate, cholesteryl palmitoleate, cholesteryl lignocerate, cholesteryl oleate, cholesteryl stearate, cholesteryl erucate, cholesterol α-linolenate, cholesteryl linoleate, cholesteryl homo-γ-linolenate, 4-hydroxy cholesterol, 6-hydroxy cholesterol, 7-hydroxy cholesterol, 19-hydroxy cholesterol, 20-hydroxy cholesterol, 22-hydroxy cholesterol, 24-hydroxy cholesterol, 25-hydroxy cholesterol, 27-hydroxy cholesterol, 27-alkyne cholesterol, 7-keto cholesterol, 7-dehydro cholesterol, 8-dehydro cholesterol, 24-dehydro cholesterol, 5α-hydroxy-6-keto cholesterol, 20,22-dihydroxy cholesterol, 7,25-dihydroxy cholesterol, 7,27-dihydroxy cholesterol, 7-keto-25-hydroxy cholesterol, fucosterol, phytosterol, cholesteryl 11,14-eicosadienoate, dimethyl hydroxyethyl aminopropane carbamoyl cholesterol iodide and mixtures thereof. The cholesterol derivative may comprise a sugar moiety such as mannose, galactose. The cholesterol derivative may comprise a sugar moiety and/or amino acids such as serine, threonine, lysine, histidine, arginine or their derivatives. The nanoparticle composition may include the cholesterol or cholesterol derivative in an amount ranging from 50 mol % to 75 mol % of cholesterol or derivative thereof per nanoparticle composition.

Pharmaceutical compositions herein may be sterilized by conventional, well-known sterilization techniques. Aqueous solutions may be packaged for use or lyophilized. The lyophilized preparation may be combined with a sterile aqueous solution prior to administration.

In an embodiment, the nanoparticle composition may include a pharmaceutically acceptable carrier. As used herein, the term “pharmaceutically-acceptable carrier” means a pharmaceutically-acceptable material, composition or vehicle, for example a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier is “acceptable” in the sense of being compatible with the other ingredients of the formulation and not injurious to the patient. Some examples of materials which may serve as pharmaceutically-acceptable carriers include: (1) sugars, for example lactose, glucose, mannose and/or sucrose; (2) starches, for example corn starch and/or potato starch; (3) cellulose, and its derivatives, for example sodium carboxymethyl cellulose, methylcellulose, ethyl cellulose, microcrystalline cellulose and/or cellulose acetate; (4) powdered tragacanth; (5) malt; (6) gelatin; (7) lubricating agents, for example magnesium stearate, sodium lauryl sulfate and/or talc; (S) excipients, for example cocoa butter and/or suppository waxes; (9) oils, for example peanut oil, cottonseed oil, safflower oil, sesame oil, olive oil, corn oil and/or soybean oil; (10) glycols, for example propylene glycol; (11) polyols, for example glycerin, sorbitol, and/or mannitol; (12) esters, for example glycerides, ethyl oleate and/or ethyl laurate; (13) agar; (14) buffering agents, for example magnesium hydroxide and/or aluminum hydroxide; (15) alginic acid; (16) pyrogen-free water; (17) diluents, for example isotonic saline, and/or PEG400; (I8) Ringer's solution; (19) C2-C12 alcohols, for example ethanol; (20) fatty acids; (21) pH buffered solutions; (22) bulking agents, for example polypeptides and/or amino acids (23) serum component, for example serum albumin, HDL and LDL; (24) surfactants, for example polysorbates (Tween 80) and/or poloxamers; and/or (25) other non-toxic compatible substances employed in pharmaceutical formulations: for example, fillers, binders, wetting agents, coloring agents, release agents, coating agents, sweetening agents, flavoring agents, perfuming agents, preservatives and/or antioxidants. The terms “excipient,” “carrier,” “pharmaceutically acceptable carrier.” or the like are used interchangeably herein.

An embodiment comprises a method for treating or preventing a disease or condition in a subject. The method may comprise providing any one of the nanoparticle compositions described herein. The method may comprise administering a therapeutically effective amount of the nanoparticle composition to a subject.

As used herein, the term “therapeutically effective amount” refers to the amount of nanoparticle composition which is effective for producing a desired therapeutic effect. The therapeutic effect may be in at least a sub-population of cells in an animal. The therapeutic effect may be achieved at a reasonable benefit/risk ratio applicable to medical treatment. A “therapeutically effective amount” may refer to an amount sufficient to generate appearance of antigen-specific antibodies in serum. A “therapeutically effective amount” may refer to an amount sufficient to cause a decrease in disease symptoms. A “therapeutically effective amount” may refer to an amount sufficient to cause a disappearance of disease symptoms. When treating viral infection, a decrease of disease symptoms may be assessed by decrease of virus in faeces, in bodily fluids, or in secreted products. The nanoparticle compositions may be administered using an amount and by a route of administration effective for generating an immune response.

Therapeutic efficacy may depend on effective amounts of active agents and time of administration necessary to achieve a desired result. Administering a nanoparticle composition may be a preventive measure. Administering of a nanoparticle composition may be a therapeutic measure to promote immunity to the infectious agent, to minimize complications associated with the slow development of immunity especially in patients with a weak immune system, elderly or infants.

The exact dosage may be chosen by the physician based on a variety of factors and in view of individual patients. Dosage and administration may be adjusted to provide sufficient levels of the active agent or agents or to maintain the desired effect. For example, factors which may be taken into account may include the type and severity of a disease; age and gender of the patient; drug combinations; and an individual response to therapy.

Therapeutic efficacy and toxicity of active agents in a nanoparticle composition may be determined by standard pharmaceutical procedures, for example, by determining the therapeutically effective dose in 50% of the population (ED50) and the lethal dose to 50% of the population (LD50) in cells cultured in vitro or experimental animals. Nanoparticle compositions may be evaluated based on the dose ratio of toxic to therapeutic effects (LD50/ED50), called the therapeutic index, the large value of which may be used for assessment. The data obtained from cell and animal studies may be used in formulating a dosage for human use.

The therapeutically effective dose may be estimated initially from cell culture assays. A therapeutically effective dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage may be monitored by a suitable bioassay.

A therapeutically effective dose may be between 0.0001 μg and 1 mg of the therapeutic or immunogenic nucleic acid per kg body weight of the subject, or between 0.00001 μg and 1 mg (μg) units/dose/subject, and may be administered on a daily basis. However, doses greater than 1 mg may be provided. For example, the amount in a dose may be at least one milligram, or about 3×1 mg, or about 10×1 mg unit of nucleic acid/dose/subject. As nanoparticle vaccines may be readily produced and inexpensively engineered and designed and stored, greater doses for large animal subjects may be economically feasible. For an animal subject several orders of magnitudes larger than the experimental animals used in examples herein, the dose may be easily adjusted, for example, the amount in a dose may be about 3×10×1 μg, or about 3×20×1 μg, or about 3×30×1 μg for animals, for example humans or small agricultural animals. However, the amount in a dose may be about 3×40×1 μg, 3×50×1 jig or even about 3×60×1 μg, for example, for a high value zoo animal or agricultural animal, for example an elephant. For preventive immunization, or periodic treatment, or treatment of a small wild animal, the amount in a dose may be less than about 3×1 μg, less than about 1 μg, less than about 500 ng, less than about 250 ng, less than about 100 ng, less than about 50 ng, less than about 25 ng, less than about 10 ng, less than about 5 ng, less than about 1 ng, less than about 500 pg, less than about 250 pg, less than about 100 pg, per dose, or in a range between any of the foregoing. The therapeutic and immunogenic nucleic acid may be a combination of different nucleic acids used per treatment dose. The terms “subject” and “individual” are used interchangeably herein, and mean a human or animal. Preferably, the animal is a vertebrate such as a primate, rodent, domestic animal or game animal. Primates include chimpanzees, cynomolgus monkeys, spider monkeys, and macaques, e.g., Rhesus. The rodent may be selected from mice, rats, guinea pigs, woodchucks, ferrets, rabbits and hamsters. The domestic or game animals may be selected from cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. A patient or subject may be selected from the foregoing or a subset of the foregoing. A patient or subject may be selected from all of the above, but excluding one or more groups or species such as humans, primates or rodents. In an embodiment, the patient or subject may be a mammal, e.g., a primate, e.g., a human. The terms, “patient” and “subject” are used interchangeably herein. The terms, “patient” and “subject” are used interchangeably herein.

Preferably, the subject is a mammal. The mammal may be a human, non-human primate, mouse, rat, dog, cat, horse, or cow, but is not limited to these examples. Mammals other than humans may be subjects that represent animal models of a disease or disorder. In addition, the methods described herein may be directed to treating domesticated animals and/or pets. A subject may be male or female.

As used herein, the terms “administer,” “administering,” “administration,” or the like refer to the placement of a composition into a subject. The administration may be by a method or route which results in at least partial localization of the composition at a desired site such that desired effect is produced. A nanoparticle composition described herein may be administered by any appropriate route known in the art including, but not limited to, oral or parenteral routes, including intravenous, intramuscular, subcutaneous, transdermal, airway (aerosol), pulmonary, nasal, rectal, or topical (including buccal and sublingual) administration.

Exemplary modes of administration include, but are not limited to, injection, infusion, instillation, inhalation, or ingestion. “Injection” includes without limitation, intravenous, intramuscular, intraarterial, intrathecal, intraventricular, intracapsular, intraorbital, intracardiac, intradermal, intraperitoneal, trans tracheal, subcutaneous, subcuticular, intraarticular, sub capsular, subarachnoid, intraspinal, intracerebral, and intrasternal injection and infusion. In an embodiment, the compositions may be administered by intravenous infusion or injection.

The nanoparticle compositions may be used for delivery of therapeutic or immunogenic nucleic acids for gene targeting. The therapeutic or immunogenic nucleic acid may be an antisense oligonucleotide (AON) or a double-stranded small interfering RNA (siRNA). Typically, siRNAs are between 21 and 23 nucleotides in length. The siRNAs may comprise a sequence complementary to a sequence contained in an mRNA transcript of a target gene when expressed within the host cell. The antisense oligonucleotide may be a morpholino antisense oligonucleotide. The antisense oligonucleotide may include a sequence complementary to a sequence contained in an mRNA transcript of a target gene. The therapeutic or immunogenic nucleic acid may be an interfering RNA (iRNA) against a specific target gene within a specific target organism. The iRNA may induce sequence-specific silencing of the expression or translation of the target polynucleotide, thereby down-regulating or preventing gene expression. The iRNA may completely inhibit expression of the target gene. The iRNA may reduce the level of expression of the target gene compared to that of an untreated control. The therapeutic or immunogenic nucleic acid may be a micro RNA (miRNA). The miRNA may be a short RNA, e.g., a hairpin RNA (hpRNA). The miRNA may be cleaved into biologically active dsRNA within the target cell by the activity of the endogenous cellular enzymes. The RNA may be a double stranded RNA (dsRNA). The ds RNA may be at least 25 nucleotides in length or may be longer. The dsRNA may contain a sequence that is complementary to the sequence of the target gene or genes.

In an embodiment, the therapeutic or immunogenic nucleic acid may be or may encode an agent that totally or partially reduces, inhibits, interferes with or modulates the activity or synthesis of one or more genes encoding target proteins. The target genes may be any genes included in the genome of a host organism. The sequence of the therapeutic or immunogenic nucleic acid may not be 100% complementary to the nucleic acid sequence of the target gene.

In an embodiment, the nanoparticle composition may be used for targeted, specific alteration of the genetic information in a subject. An embodiment comprises targeted, specific alteration of the genetic information in a subject comprising administration of a nanoparticle composition herein. As used herein, the term “alteration” refers to any change in the genome in the cells of a subject. The alteration may be insertion or deletion of nucleotides in the sequence of a target gene. “Insertion” refers to addition of one or more nucleotides to a sequence of a target gene. The term “deletion” refers to a loss or removal of one or more nucleotides in the sequence of a target gene. The alteration may be correction of the sequence of a target gene. “Correction” refers to alteration of one or more nucleotides in the sequence of a target gene, e.g., by insertion, deletion or substitution, which may result in a more favorable expression of the gene manifested by improvements in genotype and/or phenotype of the host organism.

The alteration of the genetic information may be achieved via the genome editing techniques. As used herein, “genome editing” refers to the process of modifying the nucleotide sequence in the genome in a precise or controlled manner.

An exemplary genome editing system is a Clustered Regularly Interspaced Short Palindromic Repeats (CRISPR) system as described, for example, in WO 2018/154387, which published Aug. 30, 2018 and is incorporated herein by reference as if fully set forth. In general, “CRISPR system” refers to transcripts and other elements involved in the expression of CRISPR-associated (Cas) genes, including sequences encoding a Cas gene, a tracr (trans-activating CRISPR) sequence, a tracr-mate sequence, a guide sequence, or other sequences and transcripts from a CRISPR locus. One or more tracr mate sequences may be operably linked to a guide sequence before processing or crRNA after processing by a nuclease. The tracrRNA and crRNA may be linked and may form a chimeric crRNA-tracrRNA hybrid where a mature crRNA is fused to a partial tracrRNA via a synthetic stem loop to mimic the natural crRNA:tracrRNA duplex as described in Cong et al., Science, 15:339(6121):819-823 (2013) and Jinek et al., Science, 337(6096):816-21 (2012), which are incorporated herein by reference as if fully set forth. A single fused crRNA-tracrRNA construct is also referred herein as a guide RNA or gRNA, or single-guide RNA (sgRNA). Within an sgRNA, the crRNA portion is identified as the “target sequence” and the tracrRNA is often referred to as the “scaffold.” In an embodiment, the nanoparticle compositions described herein may be used to deliver an sgRNA.

In an embodiment, the nanoparticle compositions may be used to apply other exemplary genome editing systems including meganucleases, homing endonucleases, TALEN-based systems, or Zinc Finger Nucleases. The nanoparticle compositions may be used to deliver the nucleic acid (RNA and/or DNA) that encodes the sequences for these gene editing tools, and the actual gene products, proteins, or other molecules.

In an embodiment, the nanoparticle composition may be used for gene targeting in a subject in vivo or ex vivo, e.g., by isolating cells from the subject, editing genes, and implanting the edited cells back into the subject. An embodiment comprises a method comprising administering a nanoparticle composition herein to isolated cells from a subject. The method may include gene targeting. The method may comprising implanting the edited cells back into the subject.

An embodiment comprises a method for introducing an agent into a cell. The method may comprise exposing the cell to a nanoparticle composition herein. The agent may be a nucleic acid. The agent may be one described above. The method may be a method of transfection when the agent is a nucleic acid. The agent may be introduced into cells by mixing a solution of nanoparticles composed as described herein with the liquid medium in which the cells are cultured. Examples are provided below.

The following Embodiments List includes particular embodiments of the present invention. But the list is not limiting and does not exclude alternate embodiments, or embodiments otherwise described herein.

### Embodiments List

1. A nucleic acid carrier having a structure of formula Ia or formula

Ib

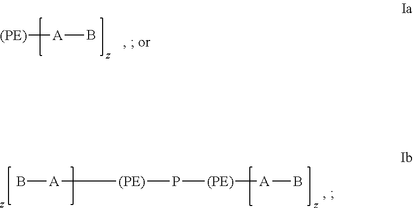

wherein PE is a polyester dendrimer or dendron which includes a core and a plurality of monomeric polyester units that form one or more generations, A is an amine linker, B is a hydrophobic unit, and z is the number of surface groups.

2. The nucleic acid carrier of embodiment 1, wherein PE has the Formula II:

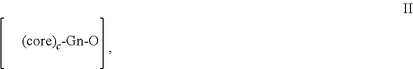

wherein c is the core multiplicity or number of wedges originating from the core, whose values independently range from 1 to 6, G is a layer or generation of dendrimer or dendron and n is a generation number and is in a range from 1 to 10.

3. The nucleic acid carrier of claim 1 or 2, wherein the monomeric polyester unit of the plurality is 2,2-bis(hydroxymethyl) propionic acid or 2,2-bis(hydroxymethyl)butyric acid.

4. The nucleic acid carrier of any one or more of embodiments 1-3, wherein z has Formula III:

z=cbⁿ, III

wherein bis branch point multiplicity, or number of branches at each branching point; c is the core multiplicity or number of wedges originating from the core and is in range from 1 to 6, and n is a generation number and is in a range from 1 to 10.

5. The nucleic acid carrier of any one or more of embodiments 1-4, wherein c is 1, and the core is a unidirectional core.

6. The nucleic acid carrier of embodiment 5, wherein the unidirectional core a carboxylic acid or derivative thereof.

7. The nucleic acid carrier of embodiment 5, wherein the core is selected from the group consisting of:

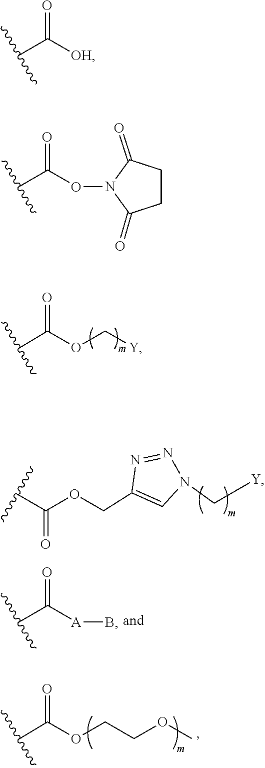

wherein Y is selected from methyl, iso-propyl, sec-butyl, iso-butyl, tert-butyl, isopentyl, neopentyl, 3-methylhexyl, 2,2-dimethylpentyl, 2,3-dimethylpentyl, azide (N3), halogen (Cl, Br, or I), acetylene (C₂H₂), hydroxyl (—OH), or thiol (—SH), -pyranosyl, cycloalkyl, aryl, heteroaryl, and heterocycle; A is an amine linker; B is a hydrophobic unit; and m is 1 to 20.

8. The nucleic acid of embodiment 7, wherein the cycloalkyl, aryl, heteroaryl, and heterocycle are substituted with at least one group selected from halogen, hydroxyl (—OH) and alkyl group.

9. The nucleic acid carrier of any or more of embodiments 1-5, wherein c is 3, and the core is a three directional core.

10. The nucleic acid carrier of embodiment 9, wherein the three directional core is trimethylol propane, or 1,1, 1-tris(hydroxyphenylethane), and has the structure of:

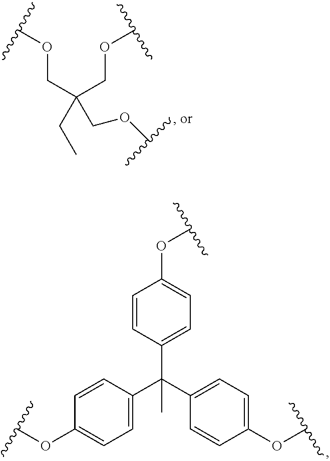 [SMILES: *OCC(CC)(CO*)CO* ; *Oc1ccc(C(C)(c2ccc(O*)cc2)c2ccc(O*)cc2)cc1]

respectively.

11. The nucleic acid carrier of any one or more of embodiments 1-5, wherein c is 4, and the core is a four directional core.

12. The nucleic acid carrier of embodiment 11, wherein the four directional core is selected from the group consisting of: pentaerythritol, adamantane-1,3,5,7-tetraol, or 5,10,15,20-Tetrakis(4-hydroxyphenyl)-21H,23H-porphine, [1,1′-biphenyl]-3,3′,5,5′-tetraol, 2,3,6,7-tetrahydroxy-9,10-dimethyl-anthracene, 3. 9,10-dimethyl-9,10-dihydro-9,10-ethanoanthracene-2,3,6,7-tetraol, 4. 6,13-dihydro-pentacene-5,7,12, 14-tetraol, Hexahydro-[1,4]dioxino[2,3-b][1,4]dioxine-2,3,6,7-tetraol, Anthracene-1,4,9,10-tetraol, pyrene-1,3,6,8-tetraol, and 3,3,3′,3′-tetramethyl-2,2′,3,3′-tetrahydro-1,1′-spirobi[indene]-5,5′,6,6′-tetrol, and has the structure of:

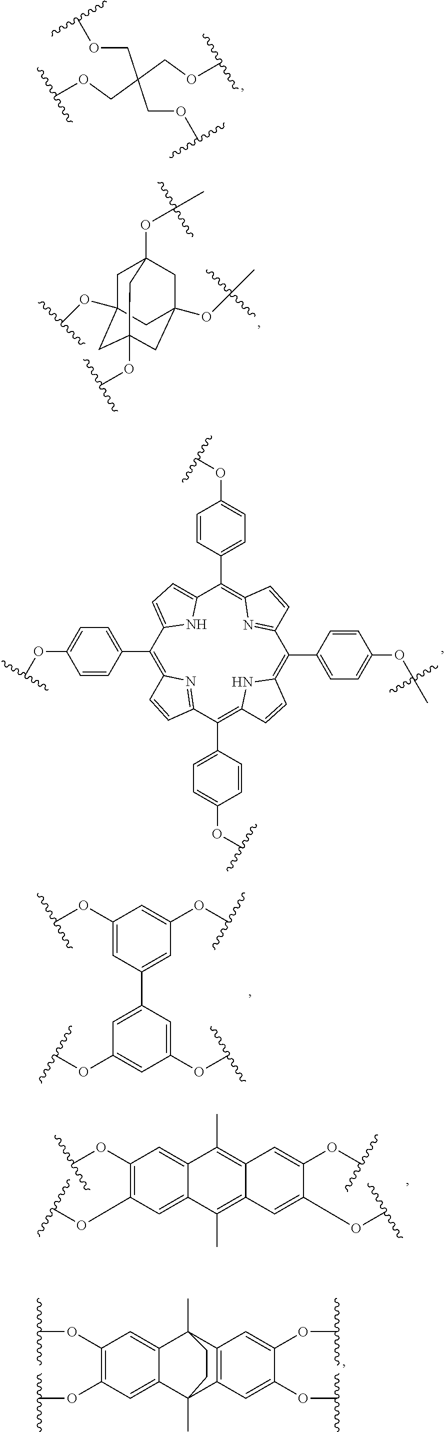

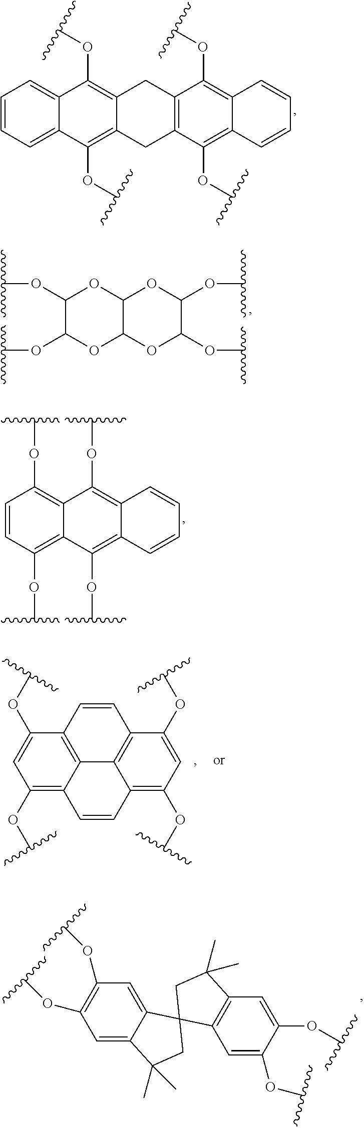 [SMILES: *Oc1c2c(c(O*)c3ccccc13)Cc1c(c(O*)c3ccccc3c1O*)C2 ; *OC1OC2OC(O*)C(O*)OC2OC1O* ; *Oc1ccc(O*)c2c(O*)c3ccccc3c(O*)c12 ; *Oc1cc(O*)c2ccc3c(O*)cc(O*)c4ccc1c2c43 ; *Oc1cc2c(cc1O*)C1(CC2(C)C)CC(C)(C)c2cc(O*)c(O*)cc21]

respectively.

13. The nucleic acid carrier of any one or more of embodiments 1-12, wherein A is derived from the group consisting of: N1-(2-aminoethyl)ethane-1,2-diamine. N1-(2-aminoethyl) propane-1,3-diamine, N1-(3-aminopropyl)propane-1,3-diamine, N1,N1′-(ethane-1,2-diyl)bis(ethane-1,2-diamine), N1,N1′-(ethane-1,2-diyl)bis(N2-(2-aminoethyl)ethane-1,2-diamine), N1-(2-(4-(2-aminoethyl)piperazin-1-yl)ethyl)ethane-1,2-diamine, N1-(2-aminoethyl)-N1-methylethane-1,2-diamine, N1-(3-aminopropyl)-N1-methylpropane-1,3-diamine, N1-(3-aminopropyl)-N1-ethylpropane-1,3-diamine, 3-((3-aminopropyl)(methyl)amino)propan-1-ol, 3,3′-(methylazanediyl)bis(propan-1-ol), N1-(3-aminopropyl)-N1-methylbutane-1,4-diamine, 4-((3-aminopropyl)(methyl)amino)butan-1-ol, 4-((3-hydroxypropyl)(methyl)amino)butan-1-ol, 4-((3-hydroxypropyl) (methyl)amino)butan-1-ol, N1-(4-aminobutyl)-N1-methylbutane-1,4-diamine, 4-((4-aminobutyl)(methyl)amino)butan-1-ol, 4,4′-(methylazanediyl)bis(butan-1-ol), 3-((3-aminopropyl) (ethyl)amino)propan-1-ol, 3,3′-(ethylazanediyl)bis(propan-1-ol), N1-(3-aminopropyl)-N1-ethylbutane-1,4-diamine, 4-((3-aminopropyl)(ethyl)amino)butan-1-ol, 4-(ethyl(3-hydroxypropyl)amino)butan-1-ol, N1-(2-aminoethyl)-N1-methylpropane-1,3-diamine, N1-(4-aminobutyl)-N1-ethylbutane-1,4-diamine, 4,4′-(ethylazanediyl) bis(butan-1-ol), 3-((3-aminopropyl)amino)propan-1-ol, N1-(3-aminopropyl)butane-1,4-diamine, 4-((3-hydroxypropyl)amino)butan-1-ol, N1-(4-aminobutyl)butane-1,4-diamine, 3,3′-azanediylbis(propan-1-ol), 4-((3-aminopropyl)amino)butan-1-ol, 4,4′-azanediylbis(butan-1-ol), and N1,N1′-(butane-1,4-diyl)bis(propane-1,3-diamine); and has the structure of:

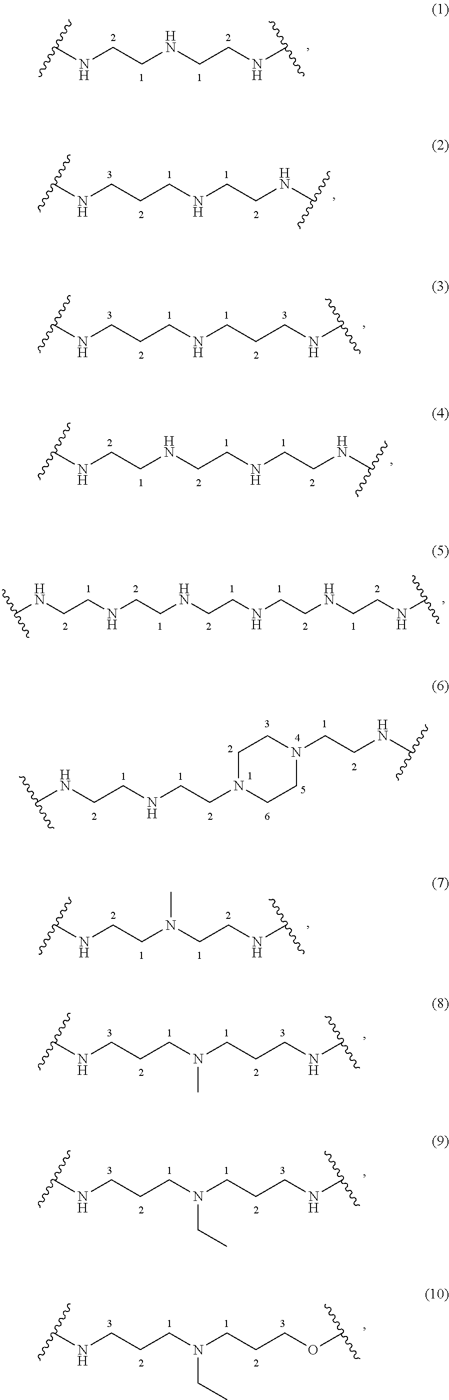

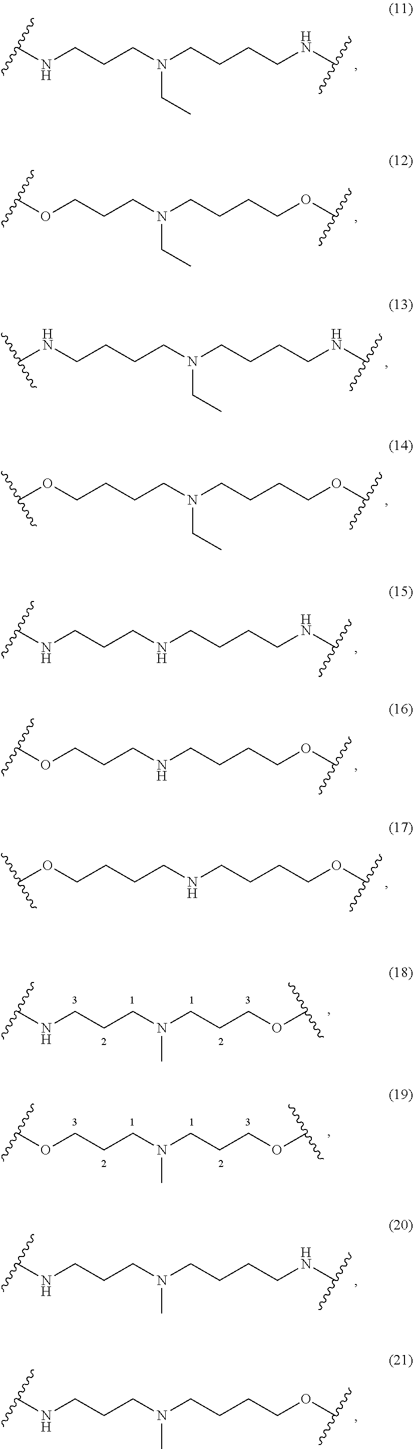

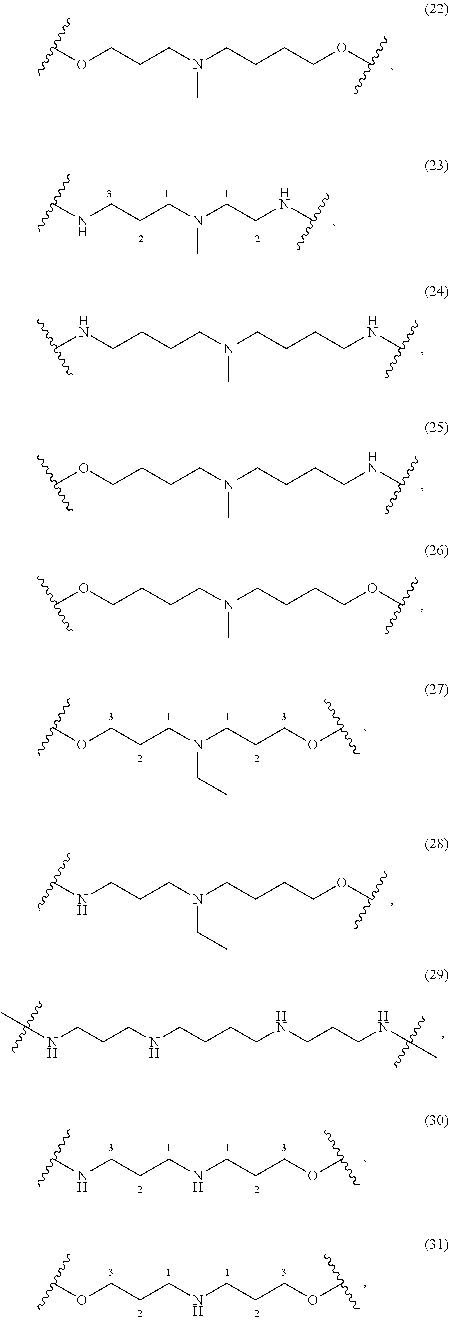

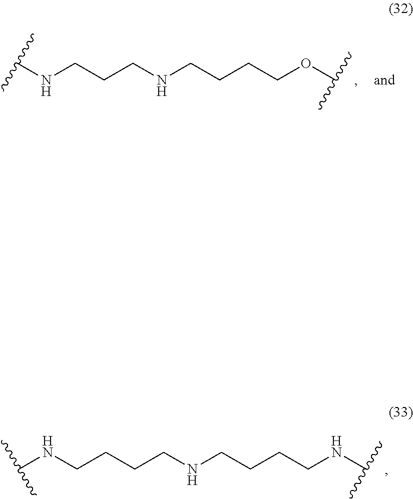 [SMILES: *NCCCNCCCCO* ; *NCCCCNCCCCN*]

respectively.

14. The nucleic acid carrier of any one or more of embodiments 1-12, wherein B is a C₁-C₂₂ alkyl or C₂-C₂₂ alkenyl group.

15. The nucleic acid carrier of embodiment 14, wherein the C₁-C₂₂ alkyl or C₂-C₂₂ alkenyl group is substituted with one to four substituents selected from the group consisting of: halogen, —CN, —NO₂, —N₃, C₁-C₆ alkyl, halo(C₁-C₆ alkyl), —OR, —NR₂, —CO₂R, —OC(O)R, —CON(R)₂, —OC(O)N(R)₂, —NHC(O)N(R)₂, —NHC(NH)N(R)₂, C₃-C₈ cycloalkyl, C₃-C₈ cycloalkenyl, aryl, heteroaryl, and heterocycle, and R is selected from the group consisting of: hydrogen, C₁-C₆ alkyl, halo(C₁-C₆ alkyl), C₃-C₈ cycloalkyl, C₃-C₈ cycloalkenyl, aryl, heteroaryl, and heterocycle.

16. The nucleic acid carrier of claim 15, wherein the one to four substituents are selected from the OR, —NR₂, —CO₂R, —OC(O)R, —CON(R)₂, —OC(O)N(R)₂, —NHC(O)N(R)₂, and —NHC(NH)N(R)₂.

17. The nucleic acid carrier of claim 15, wherein each cycloalkyl, cycloalkenyl, aryl, heteroaryl, and heterocycle is further substituted with R′ and R′ is independently selected from the group consisting of: halogen, —CN, —NO₂, —N₃, C₁-C₆ alkyl, and halo(C₁-C₆ alkyl).

18. The nucleic acid carrier of any one or more of embodiments 1-17, wherein B is an unsaturated alkyl group.

19. The nucleic acid carrier of claim 1, wherein B is selected from the group consisting of: methyl, ethyl, propyl, butyl, pentyl, hexyl, heptyl, octyl, decyl, dodecyl, tridecyl, tetradecyl, pentadecyl, hexadecyl, heptadecyl, octadecyl, but-3-en-1-yl, oct-7-en-1-yl, 12-tridecenyl, 14-pentadecenyl, 17-octadecenyl, oleyl, linoleyl, and arachidoneyl.

20. The nucleic acid carrier of any one or more of embodiments 1-17, wherein B is derived from a fatty acid or derivative thereof.

21. The nucleic acid carrier of embodiment 20, wherein the fatty acid is selected from the group consisting of: caprylic acid, capric acid, lauric acid, myristic acid, palmitic acid, stearic acid, oleic acid, linoleic acid, linolenic acid, arachidonic acid, and eicosapentaenoic acid.

22. The nucleic acid carrier of embodiment 20, wherein the fatty acid derivative is selected from the group consisting of: 12-hydroxy-9-cis-octadecenoic acid, 12-methyltetradecanoic acid, 12-methyltridecanoic acid, 14-methylhexadecanoic acid, 14-methylhexadecanoic acid, 18-methylnonadecanoic acid, 19-methylarachidic acid, isopalmitic acid, isostearic acid, phytanic acid, (±)-2-hydroxyoctanoic acid, (±)-3-hydroxydecanoic acid, (±)-3-hydroxyoctanoic acid, 10-hydroxydecanoic acid, 12-hydroxyoctadecanoic acid, 15-hydroxypentadecanoic acid, 16-hydroxyhexadecanoic acid, 2-hydroxyhexadecanoic acid, 2-hydroxytetradecanoic acid, 2-hydroxydodecanoic acid, DL-α-hydroxystearic acid, DL-β-hydroxylauric acid, DL-β-hydroxymyristic acid, and DL-β-hydroxypalmitic acid.

23. The nucleic acid carrier of any one or more of embodiments 20-22, wherein the fatty acid comprises one or more stable isotopes.

24. The nucleic acid carrier of embodiment 23, wherein the stable isotope is a stable isotope of carbon or hydrogen.

25. The nucleic acid carrier of embodiment 24, wherein the stable isotope of carbon is ¹³C.

26. The nucleic acid carrier of claim 24, wherein the stable isotope of hydrogen is ²H.

27. The nucleic acid carrier of any one or more of embodiments 23-26, wherein the fatty acid that comprises the stable isotope is selected from the group consisting of: octanoic acid-1-¹³C, octanoic acid-8-¹³C, octanoic acid-8,8,8-d3, octanoic-²H15 acid, decanoic acid-1-¹³C, decanoic acid-10-¹³C, decanoic-10,10,10-d3 acid, decanoic-d19 acid, undecanoic acid-1-¹³C, lauric acid-12,12,12-²H₃, lauric-²H23 acid, lauric acid-1-¹³C, lauric acid-1,12-¹³C₂, tridecanoic-2,2-²H2 acid, myristic acid-14-¹³C, myristic acid-1-¹³C, myristic acid-14,14,14-²H₃, myristic-²H27 acid, palmitic acid-1-¹³C, palmitic acid-16-¹³C, palmitic acid-16-¹³C,16,16,16-²H₃, palmitic acid-²H31, stearic acid-1-¹³C, stearic acid-18-¹³C, stearic acid-18,18,18-²H₃, stearic-²H35 acid, oleic acid-1-¹³C, oleic acid-²H34, linolenic acid-1-¹³C, linoleic acid-²H32, arachidonic-5,6,8,9,11,12,14,15-²H8 acid, and eicosanoic-²H39 acid.

28. The nucleic acid carrier of any one or more of embodiments 1-27, wherein P is homobifunctional linker with two azide groups, and has the structure of Formula IV:

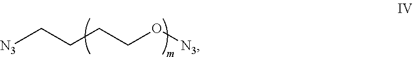

where m is the number ranging from 1 to 20.

29. A nanoparticle composition comprising the nucleic acid carrier of any one of embodiments 1-29, and a therapeutic or immunogenic nucleic acid agent enclosed therein.

30. The nanoparticle composition of embodiment 29, wherein the therapeutic or immunogenic nucleic acid agent is selected from the group consisting of: a polynucleotide, oligonucleotide, DNA, cDNA, RNA, repRNA, siRNA, miRNA, sgRNA, and mRNA.

31. The nanoparticle composition of embodiment 29 or 30, wherein the therapeutic or immunogenic nucleic acid agent encodes one or more antigens selected from the group consisting of infectious disease, pathogen, cancer, autoimmunity disease and allergenic disease.

32. The nanoparticle composition of embodiment 29 or 30, wherein the therapeutic or immunogenic nucleic acid agent comprises an RNA or DNA capable of silencing, inhibiting or modifying the activity of a gene.

33. The nanoparticle composition of any one of embodiments 29-32 further comprising a PEG-lipid.

34. The nanoparticle composition of embodiment 33, wherein the PEG-lipid is 1,2-dimyristoyl-sn-glycero-3-phosphoethanolamine-N-[methoxy (poly-ethylene glycol)-2000] or 1,2-dimyristoyl-rac-glycero-3-methoxypolyethylene glycol-2000.

35. The nanoparticle composition of embodiment 33 or 34, wherein the nanoparticle composition comprises the PEG-lipid in a range from 1 mol % to 10 mol % of the PEG-lipid per nanoparticle composition.

36. The nanoparticle composition of any one of embodiments 33-35 further comprising a phospholipid and cholesterol or derivative thereof.

37. The nanoparticle composition of embodiment 36, wherein the phospholipid is dioleoylphosphatidylcholine (DOPC) or distearoylphosphatidylcholine (DSPC).

38. The nanoparticle composition of embodiment 37, wherein the nanoparticle composition comprises the phospholipid in a range from 10 mol % to 15 mol % of the phospholipid per nanoparticle composition.

39. The nanoparticle composition of embodiment 36, wherein the nanoparticle composition comprises the cholesterol or derivative thereof in a range from 50 mol % to 75 mol % of the cholesterol or derivative thereof per nanoparticle composition.

40. A method for treating or preventing a disease or condition in a subject comprising: administering a therapeutically effective amount of the nanoparticle composition of any one of embodiments 29-39 to a subject in need thereof.

41. The method of embodiment 40, wherein the therapeutically effective amount of the nanoparticle composition comprises the therapeutic or immunogenic nucleic acid agent in a range from 0.01 mg nucleic acid to 10 mg nucleic acid per kg body weight of the subject.

42. The method of embodiment 41, wherein the subject is a mammal.

43. The method of embodiment 42, wherein the mammal is selected from the group consisting of: a chicken, a rodent, a canine, a primate, an equine, a high value agricultural animal, and a human.

### EXAMPLES

The following non-limiting examples are provided to illustrate particular embodiments. The embodiments throughout may be supplemented with one or more details from one or more examples below, and/or one or more elements from an embodiment may be substituted with one or more details from one or more examples below.

### Example 1. Nanoparticle Compositions Containing Biodegradable Dendrimers Modified with Fatty Acids

The characteristics of the surface groups of the dendrimers determine their physicochemical properties, biological activity and biocompatibility. The ideal gene delivery vehicle should be biodegradable to prevent bioaccumulation and subsequent cytotoxicity. Large dendrimers have higher packaging and thus difficult to biodegrade. There is a need for low generation dendrimers that can complex with nucleic acids and translocate across cellular membranes while maintaining biodegradability and avoiding cytotoxicity.

Preferably, the chemical bonds present on the monomeric unit are ester bonds and the terminal layer of low generation polyester dendrimers were substituted with endogenous/essential fatty acid side chains through amide bonds, rendering them susceptible to hydrolysis in plasma by esterases and amidases, and thus biodegradable. Such low generation dendrimers with fatty acid chains can be noncovalently combined with nucleic acids to form nanoparticles through their dynamic equilibrating nature.

Modified dendrimers of this example consisted of (1) bis-MPA-OH or 2,2-bis(hydroxymethyl)propionic acid dendrimers having a trimethylol propane core (generation1 and 2) or (2) a pentaerythritol core (generation1 and 2) or (3) a 1,1, 1-tris(hydroxyphenylethane) core or (4) an adamantane core as follows.

(1). bis-MPA-OH dendrimers having a trimethylol propane core (generation1 and 2):

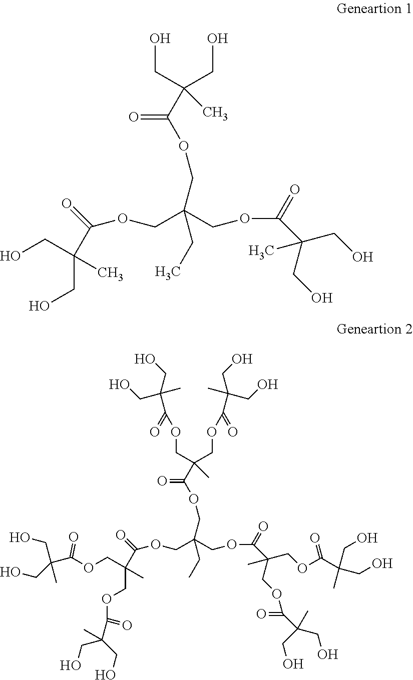 [SMILES: CCC(COC(=O)C(C)(CO)CO)(COC(=O)C(C)(CO)CO)COC(=O)C(C)(CO)CO ; CCC(COC(=O)C(C)(COC(=O)C(C)(CO)CO)COC(=O)C(C)(CO)CO)(COC(=O)C(C)(COC(=O)C(C)(CO)CO)COC(=O)C(C)(CO)CO)COC(=O)C(C)(COC(=O)C(C)(CO)CO)COC(=O)C(C)(CO)CO]

(2). bis-MPA-OH dendrimers having a pentaerythritol core (generation1 and 2):

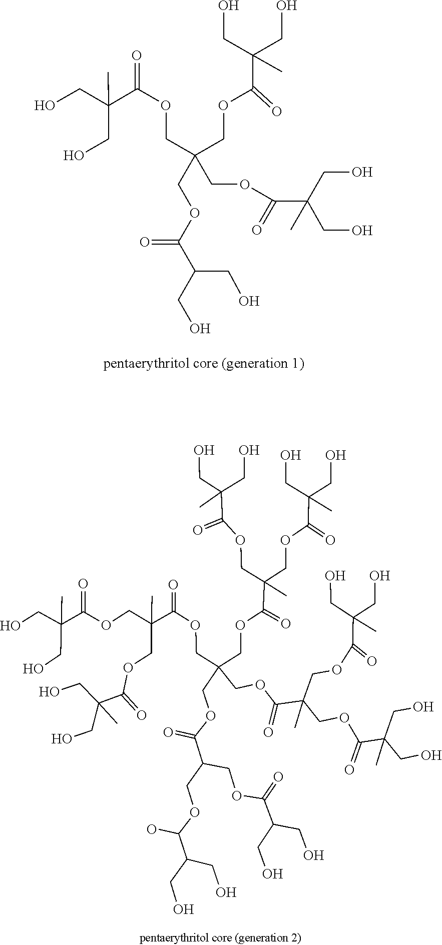

(3). bis-MPA-OH dendrimers having a 1,1,1-tris(hydroxyphenylethane) core (generation1 and 2):

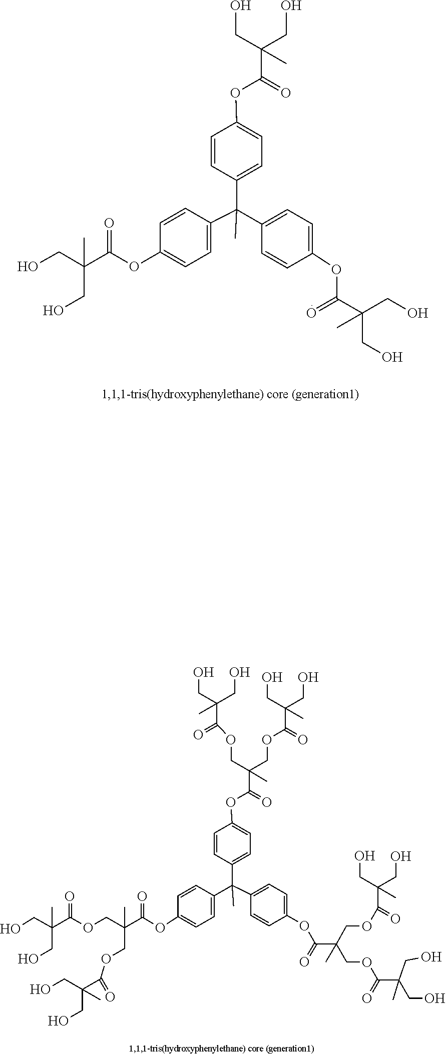

4). bis-MPA-OH dendrimers having an adamantane core (generation 1 and 2):

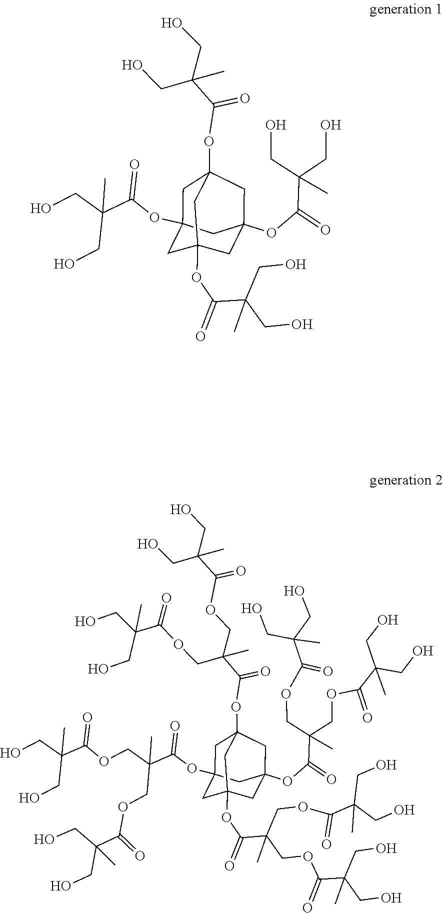 [SMILES: CC(CO)(CO)C(=O)OC12CC3(OC(=O)C(C)(CO)CO)CC(OC(=O)C(C)(CO)CO)(C1)CC(OC(=O)C(C)(CO)CO)(C2)C3 ; CC(CO)(CO)C(=O)OCC(C)(COC(=O)C(C)(CO)CO)C(=O)OC12CC3(OC(=O)C(C)(COC(=O)C(C)(CO)CO)COC(=O)C(C)(CO)CO)CC(OC(=O)C(C)(COC(=O)C(C)(CO)CO)COC(=O)C(C)(CO)CO)(C1)CC(OC(=O)C(C)(COC(=O)C(C)(CO)CO)COC(=O)C(C)(CO)CO)(C2)C3]

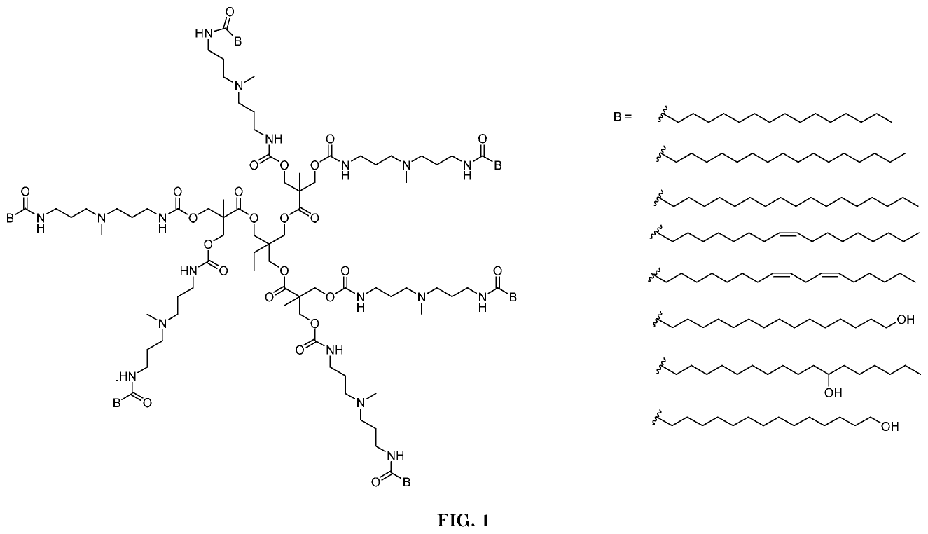

FIG. 1 is a schematic drawing of the generation 1 modified polyester dendrimer and fatty acid side chains (B) that can be used for modification. In this figure, the fatty acid side chain B can be selected from any one of C₄-C₂₈ fatty acids.

An example of the synthesis of PE-linoleic as follows:

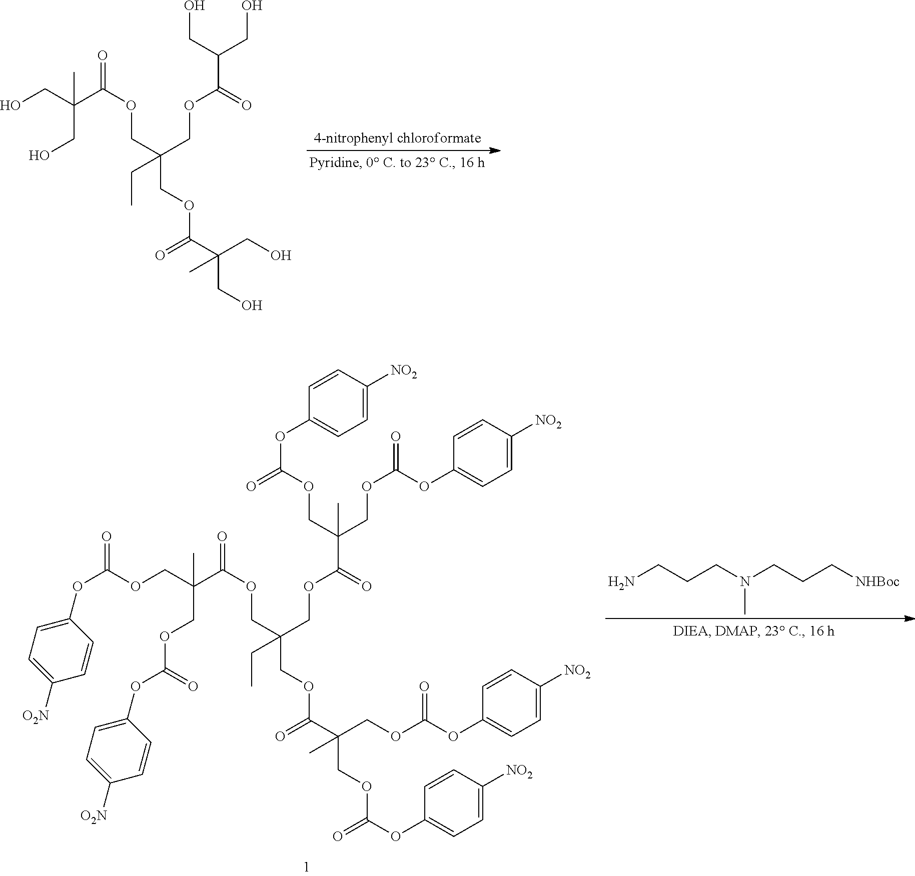 [SMILES: CCC(COC(=O)C(CO)CO)(COC(=O)C(C)(CO)CO)COC(=O)C(C)(CO)CO ; CCC(COC(=O)C(C)(COC(=O)Oc1ccc([N+](=O)[O-])cc1)COC(=O)Oc1ccc([N+](=O)[O-])cc1)(COC(=O)C(C)(COC(=O)Oc1ccc([N+](=O)[O-])cc1)COC(=O)Oc1ccc([N+](=O)[O-])cc1)COC(=O)C(C)(COC(=O)Oc1ccc([N+](=O)[O-])cc1)COC(=O)Oc1ccc([N+](=O)[O-])cc1 ; CN(CCCN)CCCNC(=O)OC(C)(C)C]

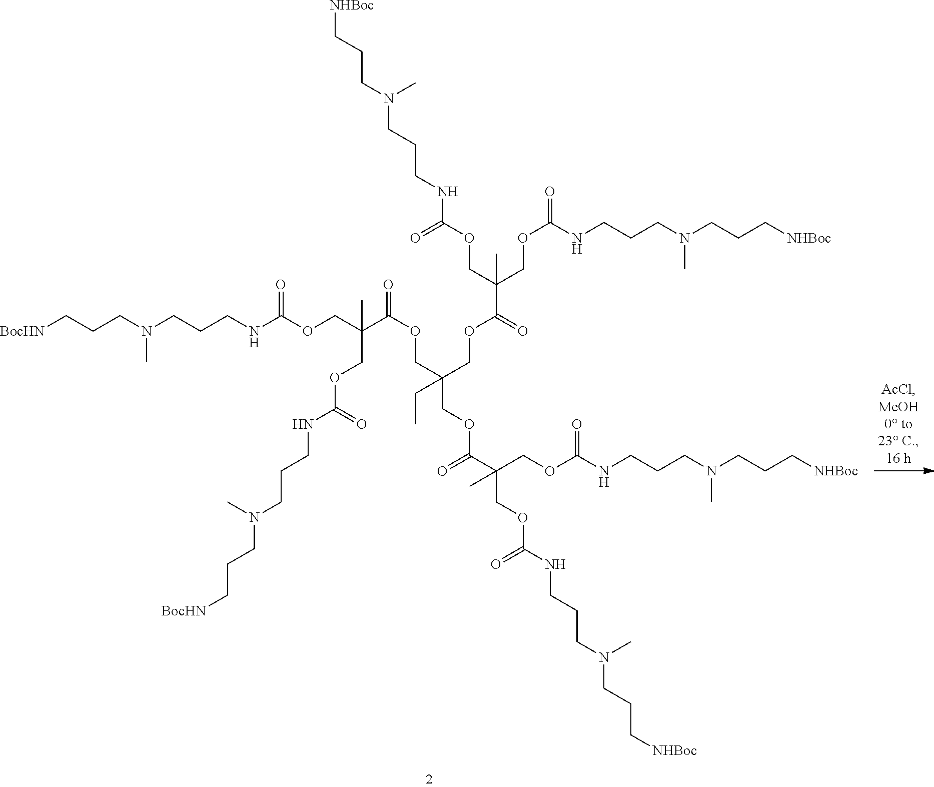 [SMILES: CCC(COC(=O)C(C)(COC(=O)NCCCN(C)CCCNC(=O)OC(C)(C)C)COC(=O)NCCCN(C)CCCNC(=O)OC(C)(C)C)(COC(=O)C(C)(COC(=O)NCCCN(C)CCCNC(=O)OC(C)(C)C)COC(=O)NCCCN(C)CCCNC(=O)OC(C)(C)C)COC(=O)C(C)(COC(=O)NCCCN(C)CCCNC(=O)OC(C)(C)C)COC(=O)NCCCN(C)CCCNC(=O)OC(C)(C)C]

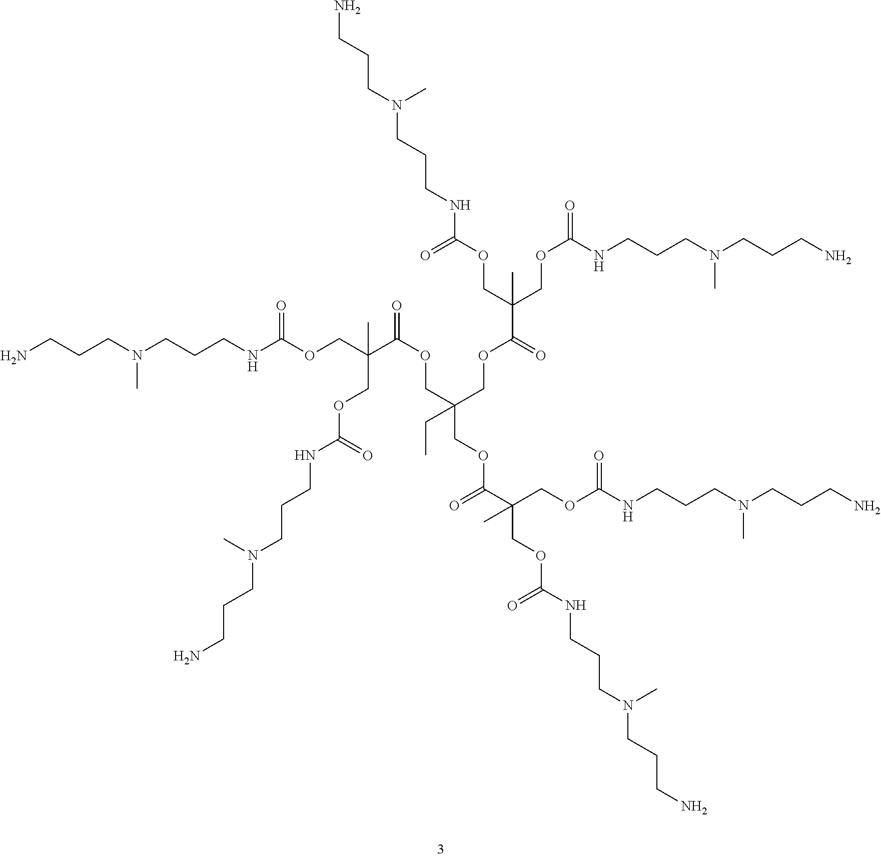 [SMILES: CCC(COC(=O)C(C)(COC(=O)NCCCN(C)CCCN)COC(=O)NCCCN(C)CCCN)(COC(=O)C(C)(COC(=O)NCCCN(C)CCCN)COC(=O)NCCCN(C)CCCN)COC(=O)C(C)(COC(=O)NCCCN(C)CCCN)COC(=O)NCCCN(C)CCCN]

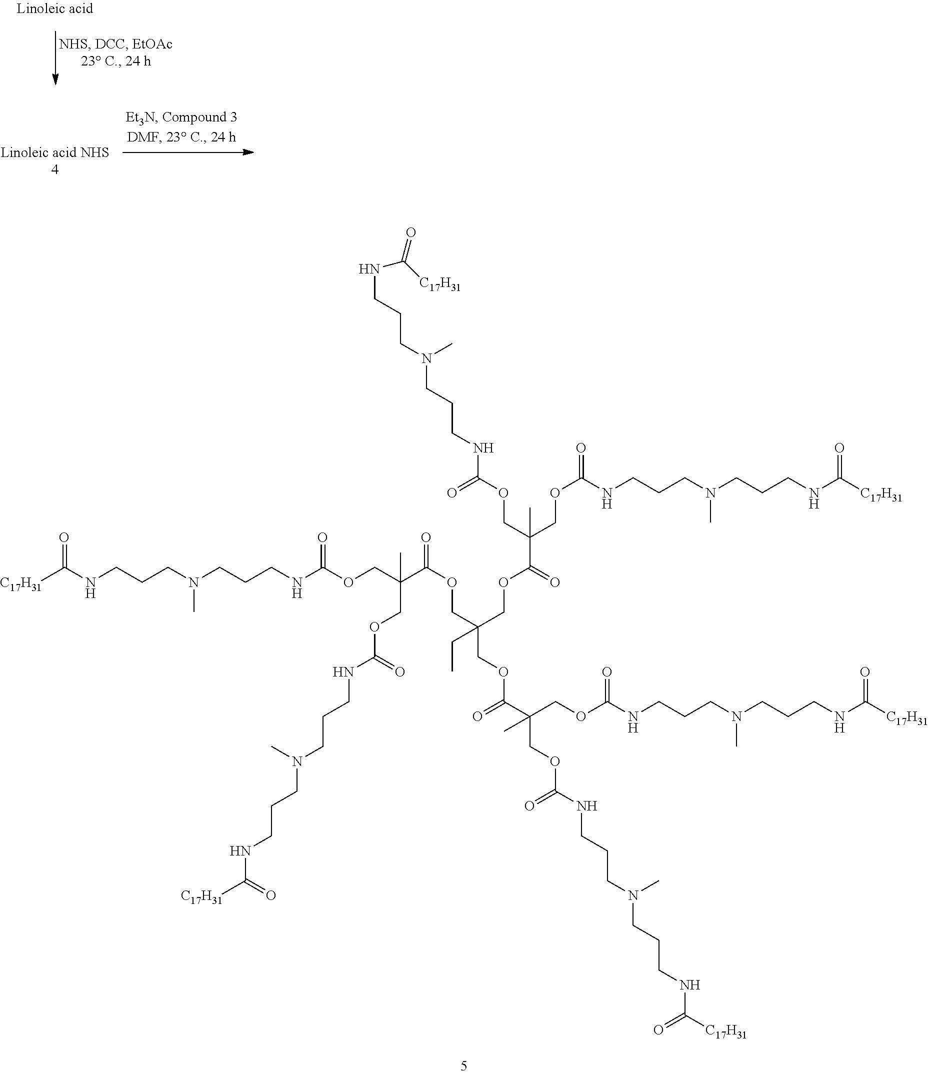

### [Synthesis Continued on Next Page]

### [Synthesis Continued on Next Page]

### [Synthesis Continued on Next Page]

Compound 1: Starting material, bis-MPA-OH dendrimer trimethylol propane core, generation 1, (300 mg, 0.62 mmol) was dissolved in dry DCM (6 mL) and pyridine (0.9 ml, 4.96 mmol) was added followed by p-nitrophenyl chloroformate (2.1 g, 10 mmol) dissolved in dry DCM (25 mL), the reaction mixture was stirred at 0° C. to room temperature (23° C.) overnight (16 hours). Next day TLC shows product formation. The reaction mixture was diluted with 1.33 M NaHSO4, and extracted with EtOAc. The organic layer was washed with brine and evaporated. Crude reaction mixture was loaded onto 40 g silica gel column. Loaded compound was then purified by flash chromatography with DCM/EtOAc. The compound started eluting at 8% EtOAc to yield the desired product as light yellow oil (870 mg, 65%). 1H NMR (301 MHz, CHLOROFORM-d) δ ppm 0.93-1.02 (m, 3H), 1.34-1.42 (m, 9H), 1.54-1.65 (m, 2H), 4.19-4.25 (m, 6H), 4.42-4.56 (m, 12H), 7.27-7.37 (m, 12H), 8.15-8.24 (m, 12H).

Compound 2: A solution of compound 1, PNP carbonate (450 mg, 0.31 mmol) dissolved in dry DCM (6 mL) was added to an excess of mono-Boc-DAPMA (453 mg, 1.85 mmol) dissolved in dry DCM (6 mL). A solution of DMAP (76 mg, 0.62 mmol) and DIPEA (0.32 ml, 1.86 mmol) in dry DCM (4 mL) was added and the reaction mixture was stirred overnight for 16 h at 23° C. under an argon atmosphere. TLC confirmed the reaction completed. The crude product was then purified by flash chromatography with mobile phase a (DCM)/mobile phase b (CH₂Cl₂/MeOH/NH₄OHaq). The compound started eluting at 40% mobile phase b (Rf=0.1 (1:1 mobile phase a/mobile phase b) to yield the desired product as light yellow oil (400 mg, 62%). 1H NMR (301 MHz, CHLOROFORM-d) 6 ppm 0.66-0.78 (m, 3H), 0.98-1.07 (m, 9H), 1.18-1.28 (m, 54H), 1.38-1.52 (m, 24H), 1.97-2.06 (m, 18H), 2.12-2.24 (m, 24H), 2.85-3.00 (m, 24H), 3.17-3.25 (m, 12H); ¹³C NMR (76 MHz, METHANOL-d4) δ ppm 17.92, 27.95, 28.04, 28.71, 39.58, 40.11, 42.18, 42.59, 48.01, 54.69, 56.01, 56.08, 64.76, 66.72, 79.66, 158.03, 158.25, 174.08.

Compound 5: 171 mg of compound 4 (0.082 mmol) was treated with 34 eq of AcCl (0.2 ml) after dissolving the compound in 3 ml MeOH, the reaction was stirred at 0° C. to 23° C. for 16 h, evaporated to dryness and dissolved in 2 ml DMF, added 0.1 ml Et₃N (0.73 mmol, 9 eq) followed by 365 mg of linoleic-NHS (as synthesized following published procedure: Talukder et al., Publication Number WO/2020/132196, which is incorporated herein by reference as if fully set forth) dissolved in 2 ml DMF. The reaction mixture was stirred at 23° C. for 24 h, concentrated under reduced pressure in Genevac, and purified via flash chromatography on silica column with gradient elution from 100% CH₂Cl₂ to 75:22:3 CH₂Cl₂/MeOH/NH₄OHaq (by volume) over 40 minutes. The desired product was eluted at 50:7:1 CH₂Cl₂/MeOH/NH₄OHaq. Fractions containing the product were combined, dried under ramping high vacuum for 12 hours to yield the desired product as light yellow oil (40 mg, 16%) and stored at 4° C. until used. ¹H NMR (300 MHz, CHLOROFORM-d) δ ppm 0.85-0.94 (m, 21H). 1.13-1.40 (m, 93H), 1.44-1.51 (m, 2H) 1.51-1.71 (m, 43H), 1.94-2.06 (m, 24H), 2.09-2.22 (m, 34H), 2.34-2.44 (m, 24H), 2.69-2.79 (m, 9H), 3.09-3.33 (m, 26H), 3.97-4.26 (m, 17H), 5.22-5.46 (m, 22H).

### Example 2. Compounds of Formula (I)

The following compounds may be prepared according to the procedures set forth above, with modifications where necessary of the starting materials to provide the desired product:

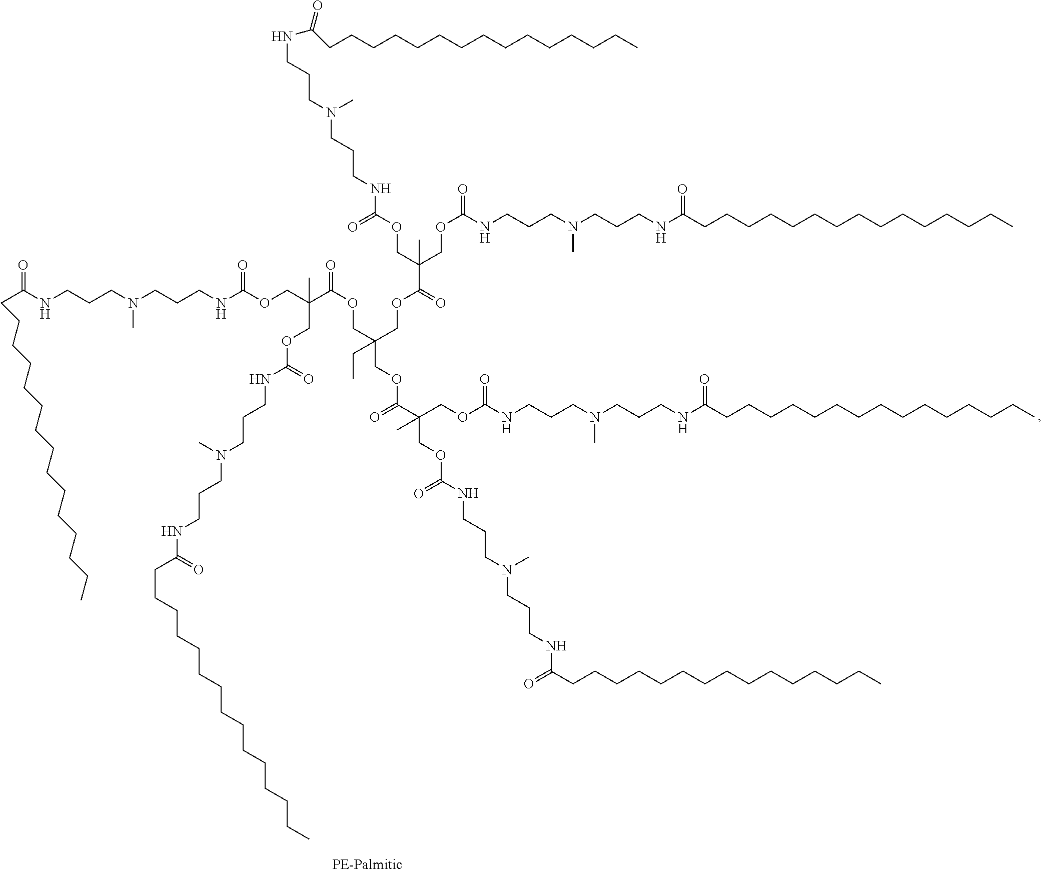 [SMILES: CCCCCCCCCCCCCCCC(=O)NCCCN(C)CCCNC(=O)OCC(C)(COC(=O)NCCCN(C)CCCNC(=O)CCCCCCCCCCCCCCC)C(=O)OCC(CC)(COC(=O)C(C)(COC(=O)NCCCN(C)CCCNC(=O)CCCCCCCCCCCCCCC)COC(=O)NCCCN(C)CCCNC(=O)CCCCCCCCCCCCCCC)COC(=O)C(C)(COC(=O)NCCCN(C)CCCNC(=O)CCCCCCCCCCCCCCC)COC(=O)NCCCN(C)CCCNC(=O)CCCCCCCCCCCCCCC]

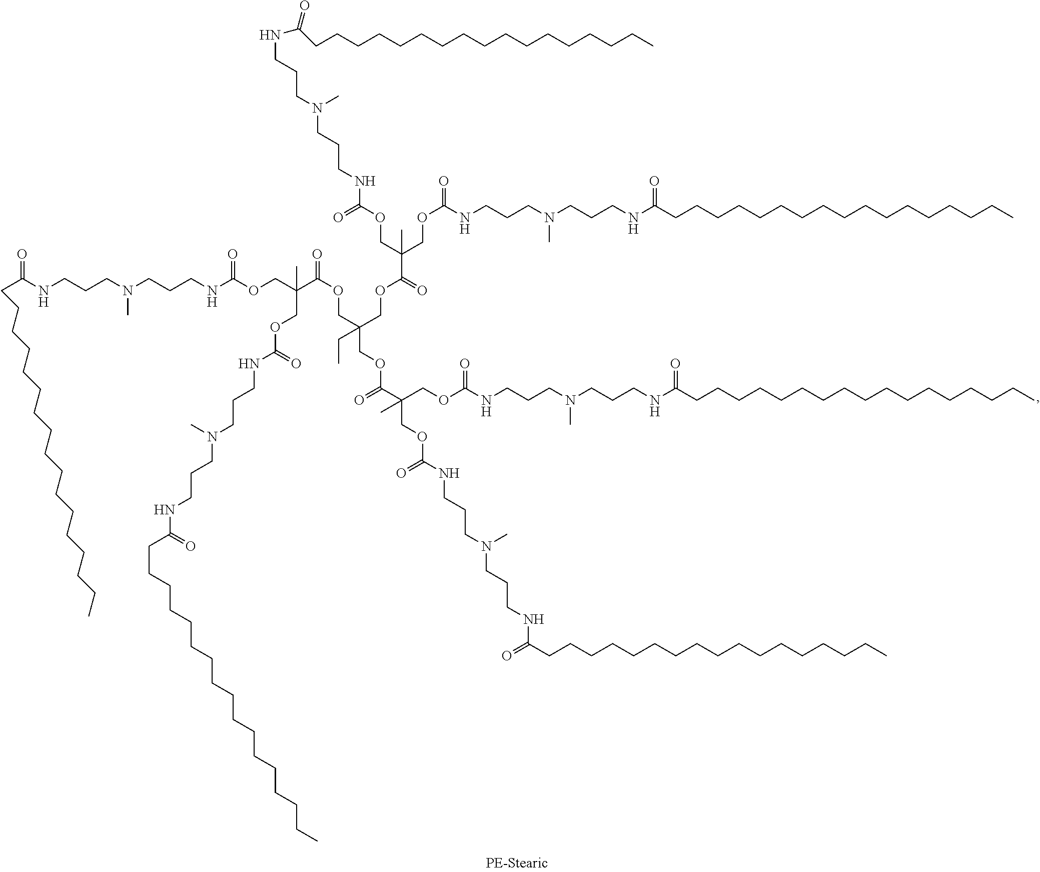 [SMILES: CCCCCCCCCCCCCCCCCC(=O)NCCCN(C)CCCNC(=O)OCC(C)(COC(=O)NCCCN(C)CCCNC(=O)CCCCCCCCCCCCCCCCC)C(=O)OCC(CC)(COC(=O)C(C)(COC(=O)NCCCN(C)CCCNC(=O)CCCCCCCCCCCCCCCCC)COC(=O)NCCCN(C)CCCNC(=O)CCCCCCCCCCCCCCCCC)COC(=O)C(C)(COC(=O)NCCCN(C)CCCNC(=O)CCCCCCCCCCCCCCCCC)COC(=O)NCCCN(C)CCCNC(=O)CCCCCCCCCCCCCCCCC]

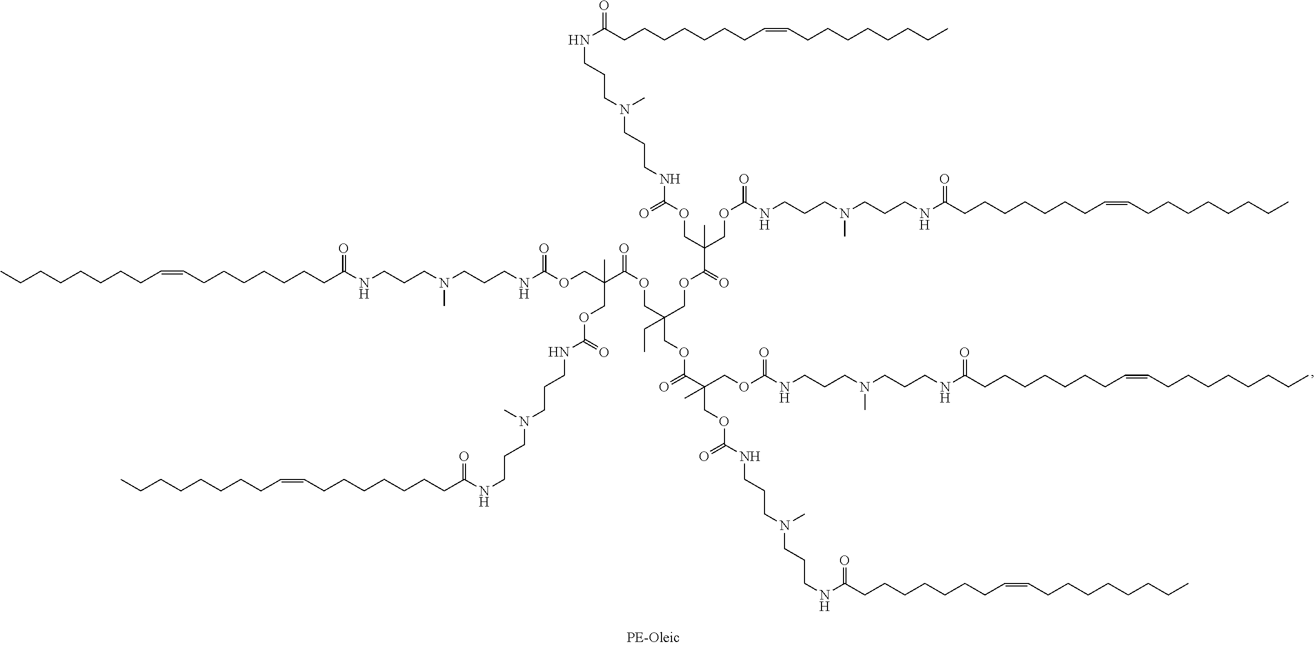 [SMILES: CCCCCCCC/C=C\CCCCCCCC(=O)NCCCN(C)CCCNC(=O)OCC(C)(COC(=O)NCCCN(C)CCCNC(=O)CCCCCCC/C=C\CCCCCCCC)C(=O)OCC(CC)(COC(=O)C(C)(COC(=O)NCCCN(C)CCCNC(=O)CCCCCCC/C=C\CCCCCCCC)COC(=O)NCCCN(C)CCCNC(=O)CCCCCCC/C=C\CCCCCCCC)COC(=O)C(C)(COC(=O)NCCCN(C)CCCNC(=O)CCCCCCC/C=C\CCCCCCCC)COC(=O)NCCCN(C)CCCNC(=O)CCCCCCC/C=C\CCCCCCCC]

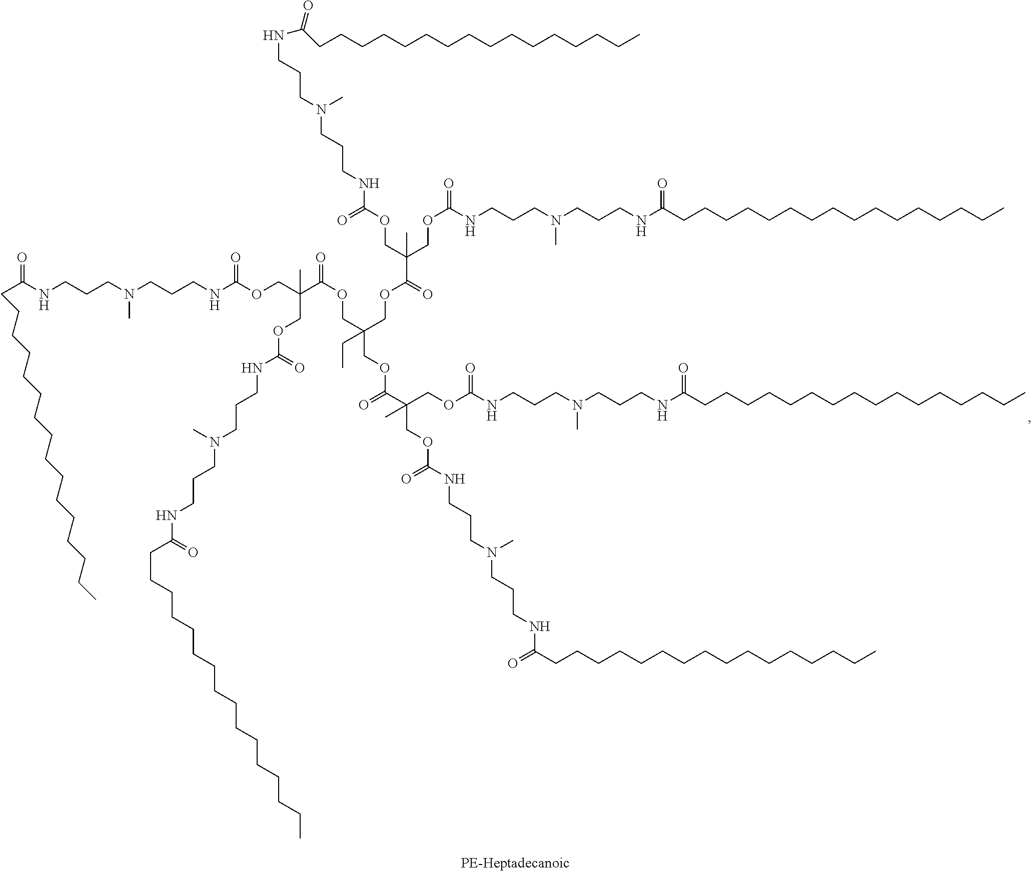 [SMILES: CCCCCCCCCCCCCCCCC(=O)NCCCN(C)CCCNC(=O)OCC(C)(COC(=O)NCCCN(C)CCCNC(=O)CCCCCCCCCCCCCCCC)C(=O)OCC(CC)(COC(=O)C(C)(COC(=O)NCCCN(C)CCCNC(=O)CCCCCCCCCCCCCCCC)COC(=O)NCCCN(C)CCCNC(=O)CCCCCCCCCCCCCCCC)COC(=O)C(C)(COC(=O)NCCCN(C)CCCNC(=O)CCCCCCCCCCCCCCCC)COC(=O)NCCCN(C)CCCNC(=O)CCCCCCCCCCCCCCCC]

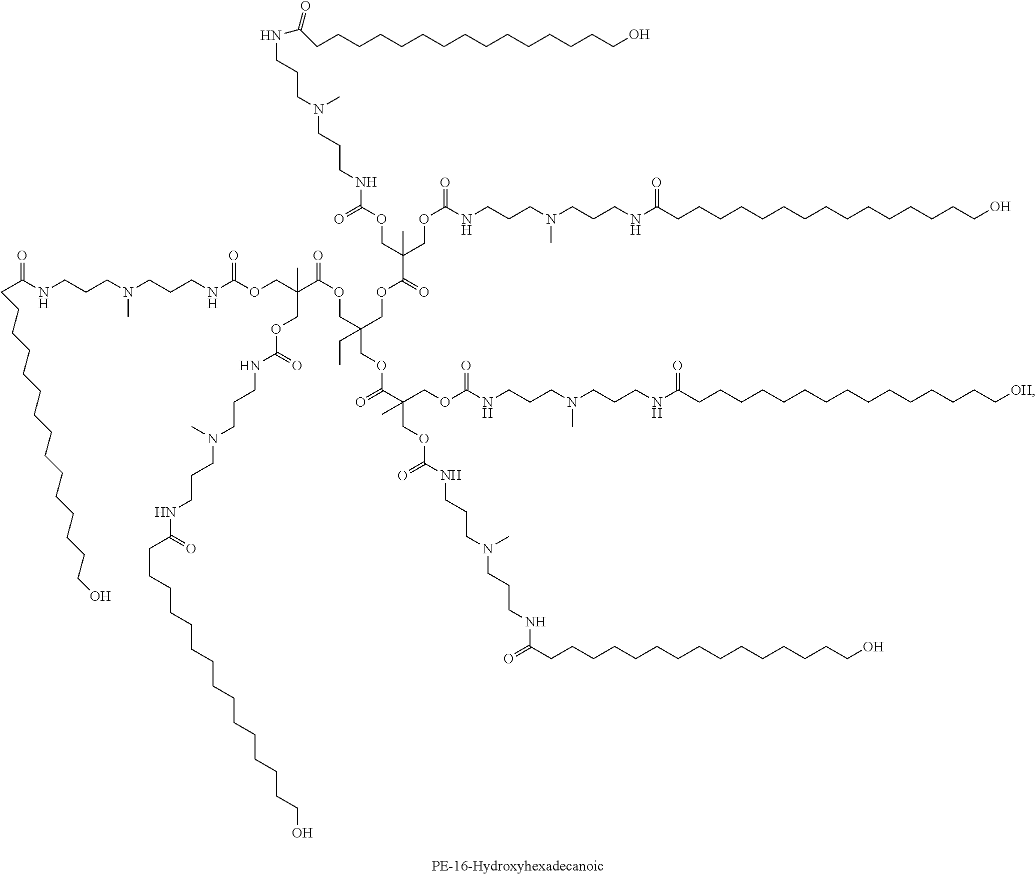 [SMILES: CCC(COC(=O)C(C)(COC(=O)NCCCN(C)CCCNC(=O)CCCCCCCCCCCCCCCO)COC(=O)NCCCN(C)CCCNC(=O)CCCCCCCCCCCCCCCO)(COC(=O)C(C)(COC(=O)NCCCN(C)CCCNC(=O)CCCCCCCCCCCCCCCO)COC(=O)NCCCN(C)CCCNC(=O)CCCCCCCCCCCCCCCO)COC(=O)C(C)(COC(=O)NCCCN(C)CCCNC(=O)CCCCCCCCCCCCCCCO)COC(=O)NCCCN(C)CCCNC(=O)CCCCCCCCCCCCCCCO]

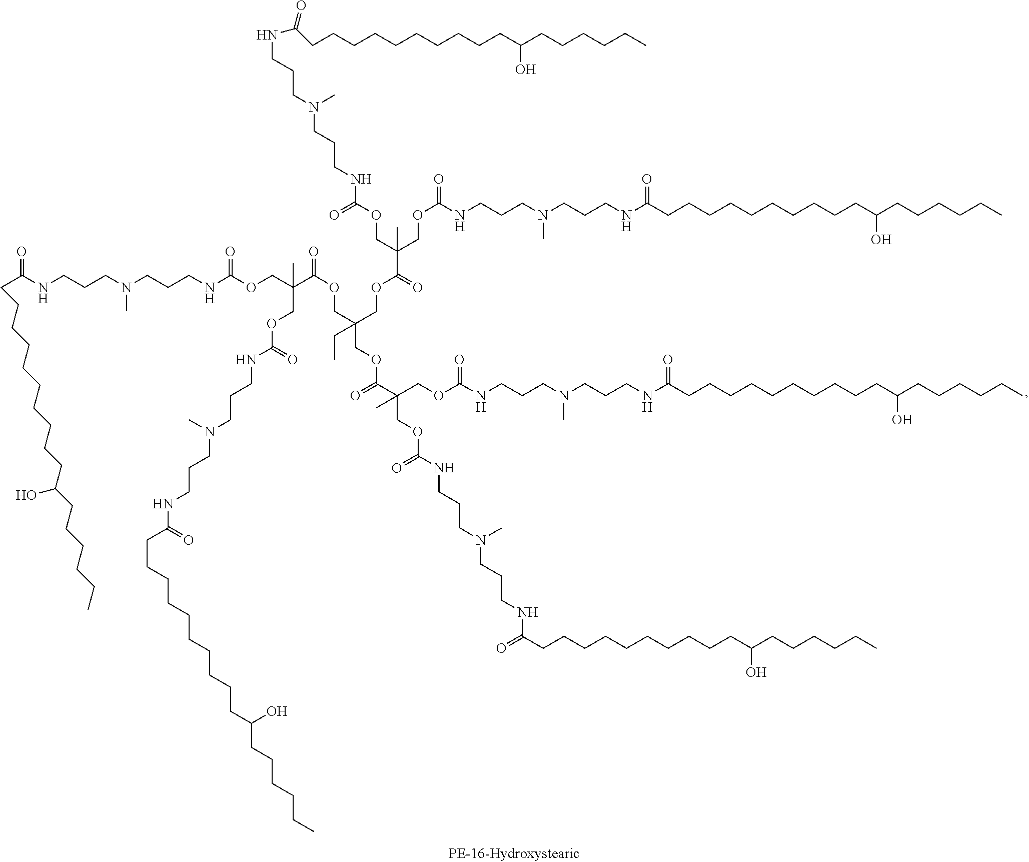 [SMILES: CCCCCCC(O)CCCCCCCCCCC(=O)NCCCN(C)CCCNC(=O)OCC(C)(COC(=O)NCCCN(C)CCCNC(=O)CCCCCCCCCCC(O)CCCCCC)C(=O)OCC(CC)(COC(=O)C(C)(COC(=O)NCCCN(C)CCCNC(=O)CCCCCCCCCCC(O)CCCCCC)COC(=O)NCCCN(C)CCCNC(=O)CCCCCCCCCCC(O)CCCCCC)COC(=O)C(C)(COC(=O)NCCCN(C)CCCNC(=O)CCCCCCCCCCC(O)CCCCCC)COC(=O)NCCCN(C)CCCNC(=O)CCCCCCCCCCC(O)CCCCCC]

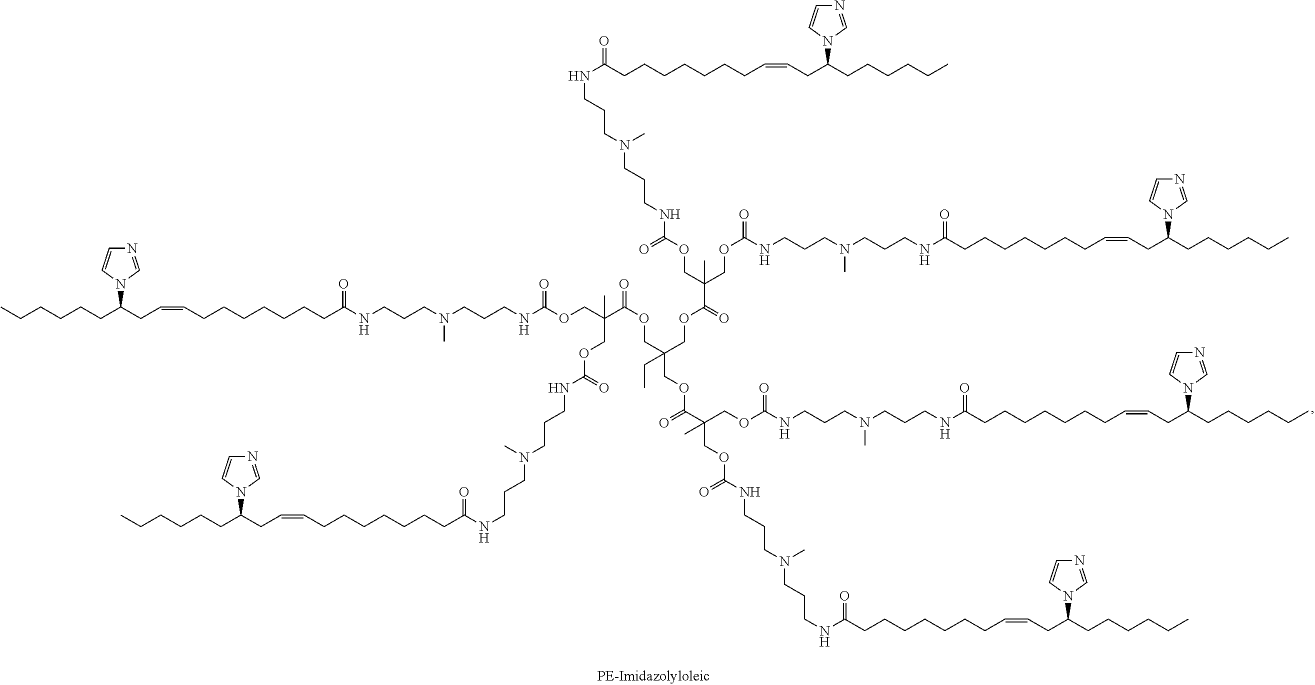 [SMILES: CCCCCC[C@@H](C/C=C\CCCCCCCC(=O)NCCCN(C)CCCNC(=O)OCC(C)(COC(=O)NCCCN(C)CCCNC(=O)CCCCCCC/C=C\C[C@H](CCCCCC)n1ccnc1)C(=O)OCC(CC)(COC(=O)C(C)(COC(=O)NCCCN(C)CCCNC(=O)CCCCCCC/C=C\C[C@H](CCCCCC)n1ccnc1)COC(=O)NCCCN(C)CCCNC(=O)CCCCCCC/C=C\C[C@H](CCCCCC)n1ccnc1)COC(=O)C(C)(COC(=O)NCCCN(C)CCCNC(=O)CCCCCCC/C=C\C[C@@H](CCCCCC)n1ccnc1)COC(=O)NCCCN(C)CCCNC(=O)CCCCCCC/C=C\C[C@@H](CCCCCC)n1ccnc1)n1ccnc1]

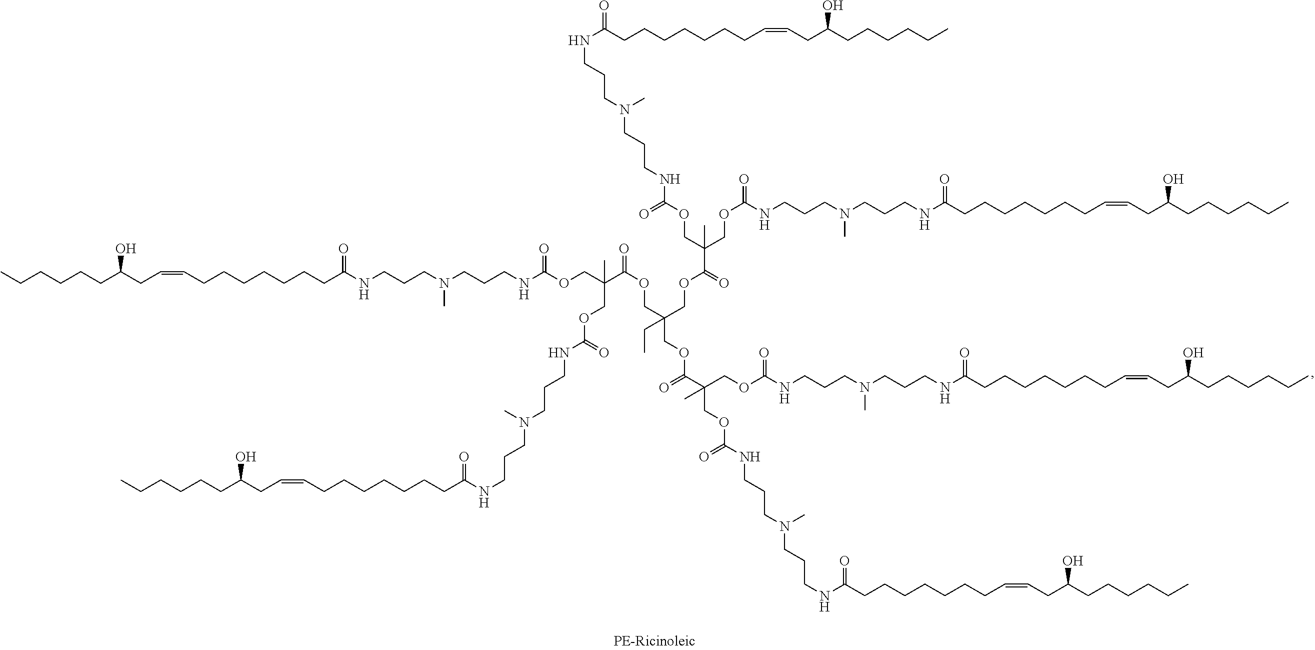 [SMILES: CCCCCC[C@H](O)C/C=C\CCCCCCCC(=O)NCCCN(C)CCCNC(=O)OCC(C)(COC(=O)NCCCN(C)CCCNC(=O)CCCCCCC/C=C\C[C@@H](O)CCCCCC)C(=O)OCC(CC)(COC(=O)C(C)(COC(=O)NCCCN(C)CCCNC(=O)CCCCCCC/C=C\C[C@@H](O)CCCCCC)COC(=O)NCCCN(C)CCCNC(=O)CCCCCCC/C=C\C[C@@H](O)CCCCCC)COC(=O)C(C)(COC(=O)NCCCN(C)CCCNC(=O)CCCCCCC/C=C\C[C@H](O)CCCCCC)COC(=O)NCCCN(C)CCCNC(=O)CCCCCCC/C=C\C[C@H](O)CCCCCC]

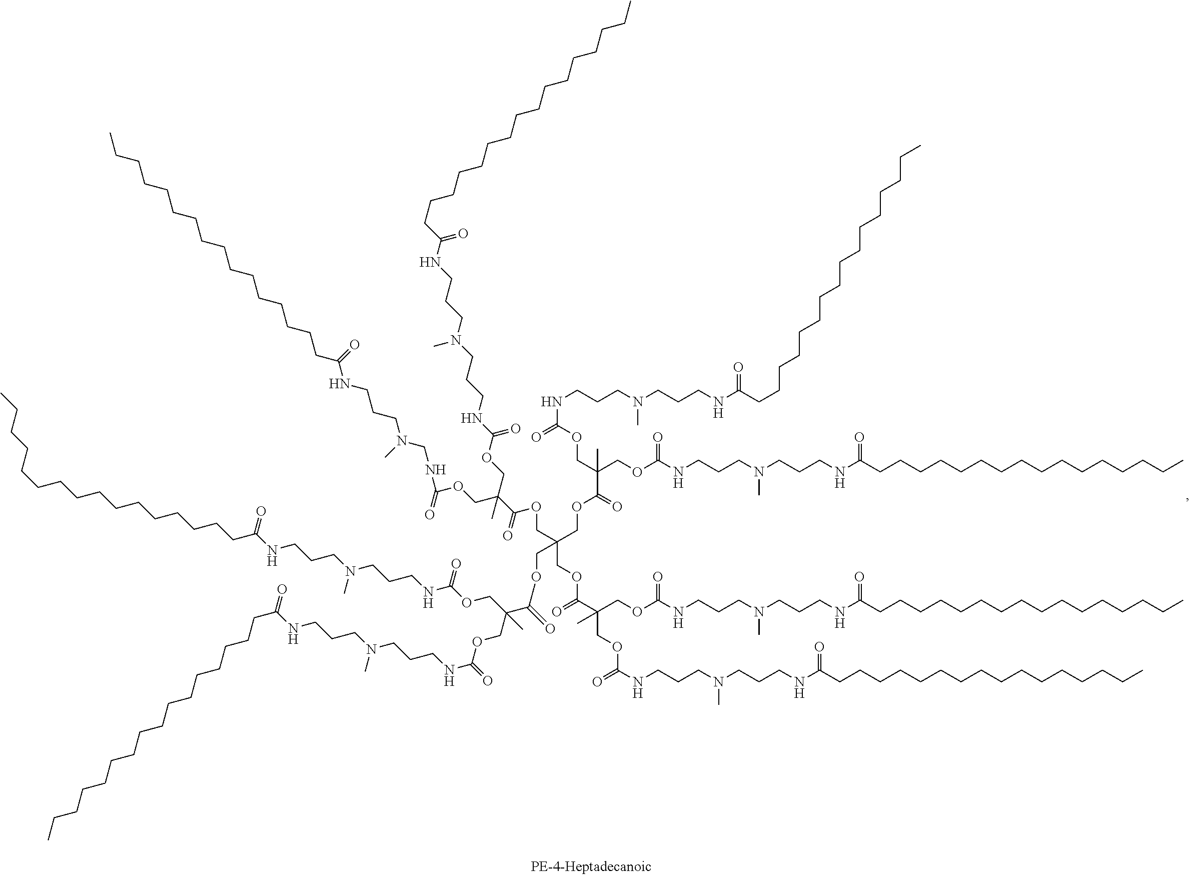 [SMILES: CCCCCCCCCCCCCCCCC(=O)NCCCN(C)CCCNC(=O)OCC(C)(COC(=O)NCCCN(C)CCCNC(=O)CCCCCCCCCCCCCCCC)C(=O)OCC(COC(=O)C(C)(COC(=O)NCCCN(C)CCCNC(=O)CCCCCCCCCCCCCCCC)COC(=O)NCCCN(C)CCCNC(=O)CCCCCCCCCCCCCCCC)(COC(=O)C(C)(COC(=O)NCCCN(C)CCCNC(=O)CCCCCCCCCCCCCCCC)COC(=O)NCCCN(C)CCCNC(=O)CCCCCCCCCCCCCCCC)COC(=O)C(C)(COC(=O)NCCCN(C)CCCNC(=O)CCCCCCCCCCCCCCCC)COC(=O)NCN(C)CCCNC(=O)CCCCCCCCCCCCCCCC]

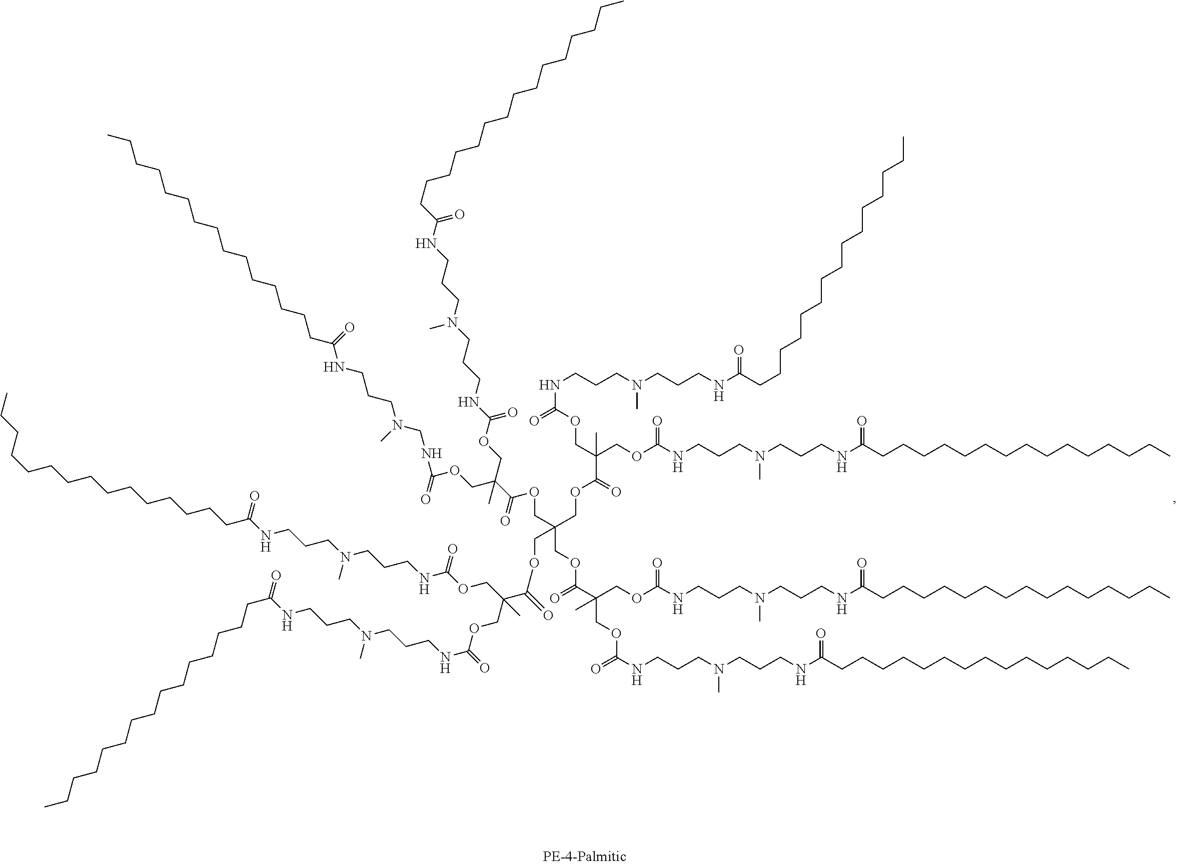 [SMILES: CCCCCCCCCCCCCCCC(=O)NCCCN(C)CCCNC(=O)OCC(C)(COC(=O)NCCCN(C)CCCNC(=O)CCCCCCCCCCCCCCC)C(=O)OCC(COC(=O)C(C)(COC(=O)NCCCN(C)CCCNC(=O)CCCCCCCCCCCCCCC)COC(=O)NCCCN(C)CCCNC(=O)CCCCCCCCCCCCCCC)(COC(=O)C(C)(COC(=O)NCCCN(C)CCCNC(=O)CCCCCCCCCCCCCCC)COC(=O)NCCCN(C)CCCNC(=O)CCCCCCCCCCCCCCC)COC(=O)C(C)(COC(=O)NCCCN(C)CCCNC(=O)CCCCCCCCCCCCCCC)COC(=O)NCN(C)CCCNC(=O)CCCCCCCCCCCCCCC]

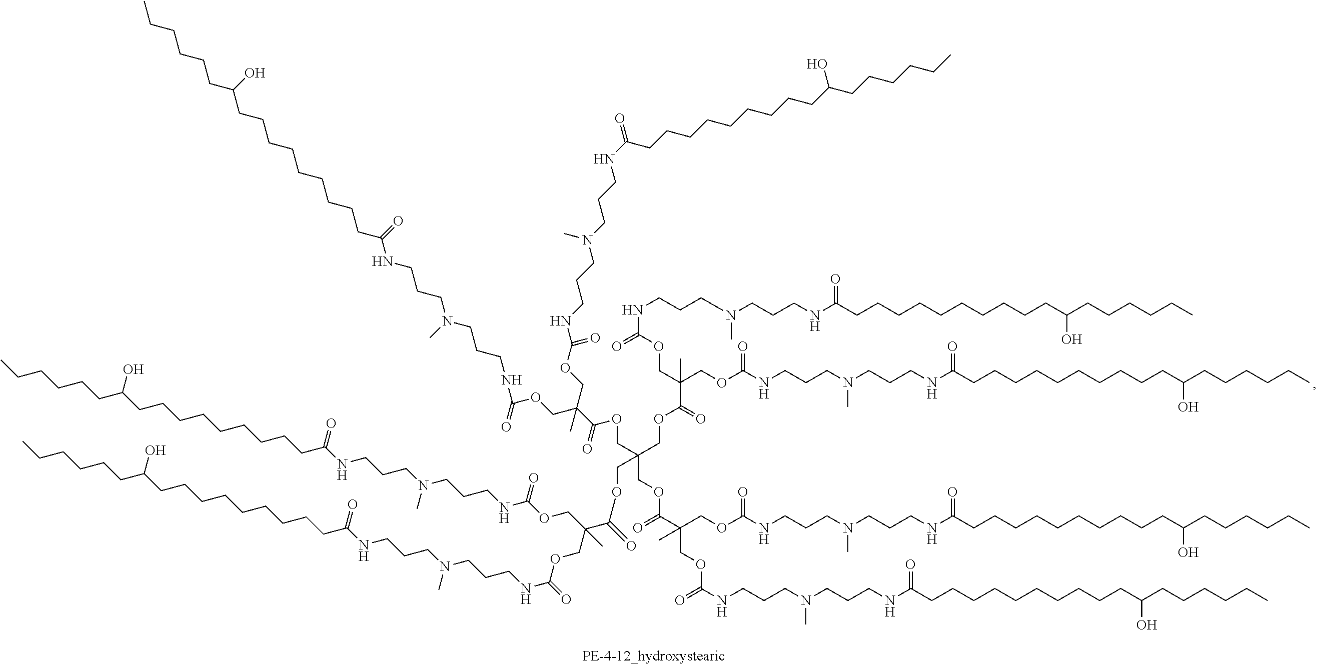 [SMILES: CCCCCCC(O)CCCCCCCCCCC(=O)NCCCN(C)CCCNC(=O)OCC(C)(COC(=O)NCCCN(C)CCCNC(=O)CCCCCCCCCCC(O)CCCCCC)C(=O)OCC(COC(=O)C(C)(COC(=O)NCCCN(C)CCCNC(=O)CCCCCCCCCCC(O)CCCCCC)COC(=O)NCCCN(C)CCCNC(=O)CCCCCCCCCCC(O)CCCCCC)(COC(=O)C(C)(COC(=O)NCCCN(C)CCCNC(=O)CCCCCCCCCC(O)CCCCCC)COC(=O)NCCCN(C)CCCNC(=O)CCCCCCCCCC(O)CCCCCC)COC(=O)C(C)(COC(=O)NCCCN(C)CCCNC(=O)CCCCCCCCCC(O)CCCCCC)COC(=O)NCCCN(C)CCCNC(=O)CCCCCCCCCC(O)CCCCCC]

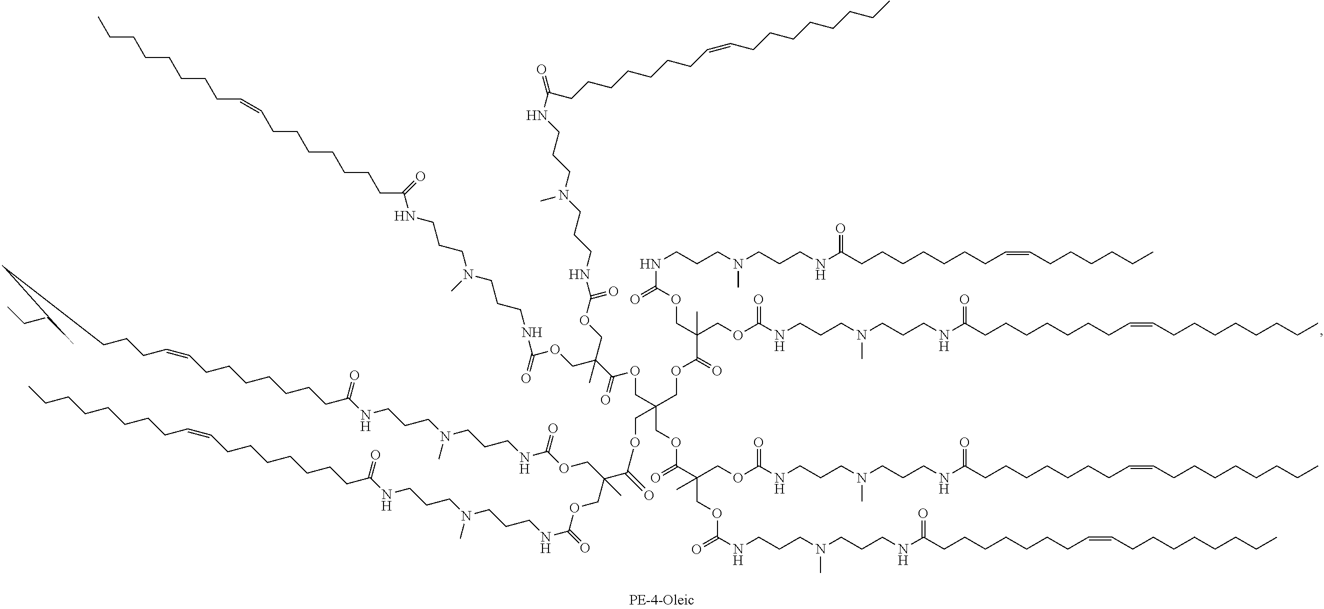 [SMILES: CCCCCC/C=C\CCCCCCCC(=O)NCCCN(C)CCCNC(=O)OCC(C)(COC(=O)NCCCN(C)CCCNC(=O)CCCCCCC/C=C\CCCCCCCC)C(=O)OCC(COC(=O)C(C)(COC(=O)NCCCN(C)CCCNC(=O)CCCCCCC/C=C\CCCCCCCC)COC(=O)NCCCN(C)CCCNC(=O)CCCCCCC/C=C\CCCCCCCC)(COC(=O)C(C)(COC(=O)NCCCN(C)CCCNC(=O)CCCCCCC/C=C\CCCCCCCC)COC(=O)NCCCN(C)CCCNC(=O)CCCCCCC/C=C\CCCCCCCC)COC(=O)C(C)(COC(=O)NCCCN(C)CCCNC(=O)CCCCCCC/C=C\CCCCCCCC)COC(=O)NCCCN(C)CCCNC(=O)CCCCCCC/C=C\CCCCCCCC]

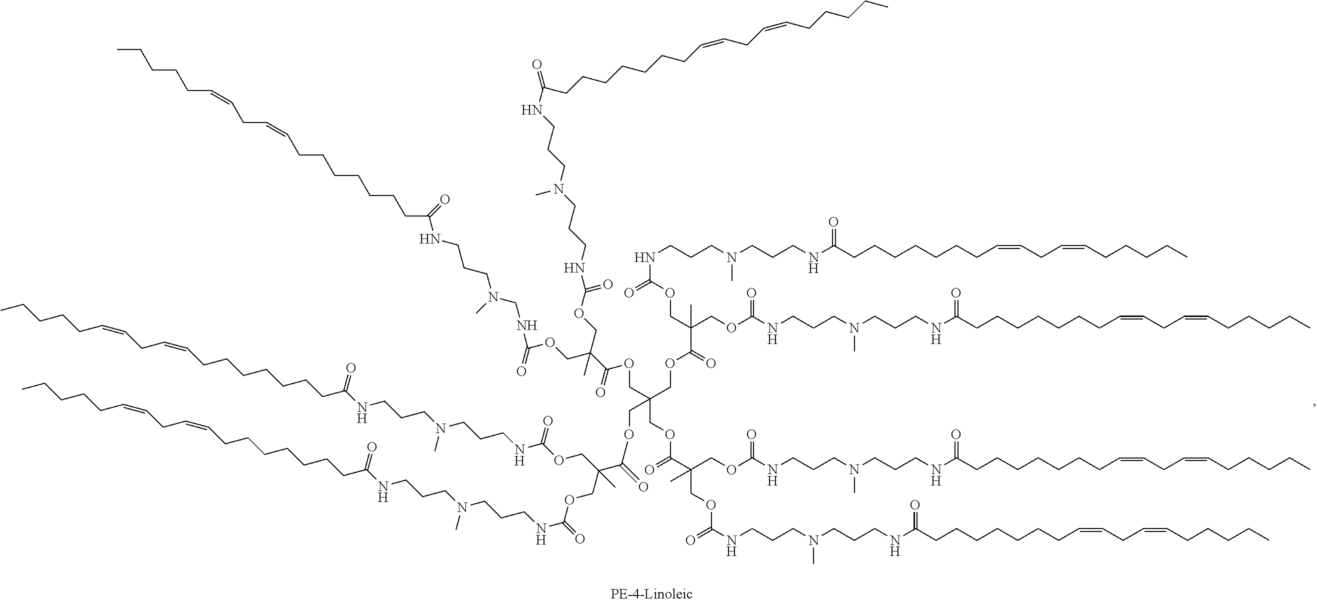 [SMILES: CCCCC/C=C\C/C=C\CCCCCCCC(=O)NCCCN(C)CCCNC(=O)OCC(C)(COC(=O)NCCCN(C)CCCNC(=O)CCCCCCC/C=C\C/C=C\CCCCC)C(=O)OCC(COC(=O)C(C)(COC(=O)NCCCN(C)CCCNC(=O)CCCCCCC/C=C\C/C=C\CCCCC)COC(=O)NCCCN(C)CCCNC(=O)CCCCCCC/C=C\C/C=C\CCCCC)(COC(=O)C(C)(COC(=O)NCCCN(C)CCCNC(=O)CCCCCCC/C=C\C/C=C\CCCCC)COC(=O)NCCCN(C)CCCNC(=O)CCCCCCC/C=C\C/C=C\CCCCC)COC(=O)C(C)(COC(=O)NCCCN(C)CCCNC(=O)CCCCCCC/C=C\C/C=C\CCCCC)COC(=O)NCN(C)CCCNC(=O)CCCCCCC/C=C\C/C=C\CCCCC]

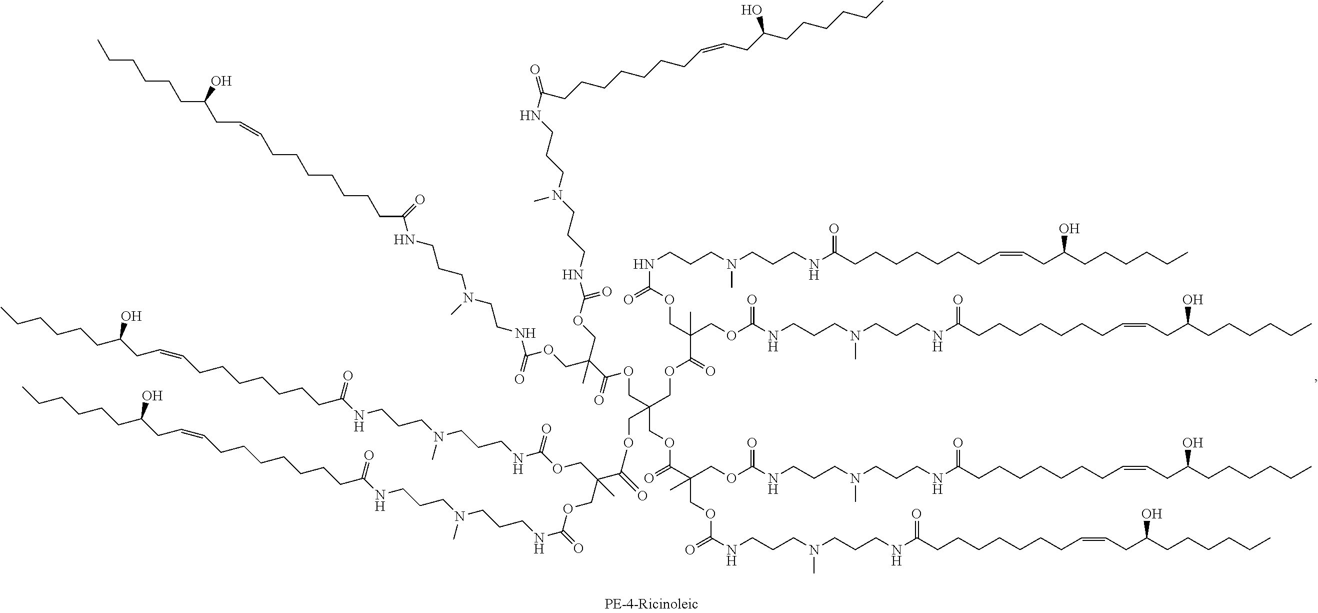 [SMILES: CCCCCC[C@H](O)C/C=C\CCCCCCCC(=O)NCCCN(C)CCCNC(=O)OCC(C)(COC(=O)NCCCN(C)CCCNC(=O)CCCCCCC/C=C\C[C@@H](O)CCCCCC)C(=O)OCC(COC(=O)C(C)(COC(=O)NCCCN(C)CCCNC(=O)CCCCCCC/C=C\C[C@@H](O)CCCCCC)COC(=O)NCCCN(C)CCCNC(=O)CCCCCCC/C=C\C[C@@H](O)CCCCCC)(COC(=O)C(C)(COC(=O)NCCCN(C)CCCNC(=O)CCCCCCC/C=C\C[C@H](O)CCCCCC)COC(=O)NCCCN(C)CCCNC(=O)CCCCCCC/C=C\C[C@H](O)CCCCCC)COC(=O)C(C)(COC(=O)NCCCN(C)CCCNC(=O)CCCCCCC/C=C\C[C@@H](O)CCCCCC)COC(=O)NCCN(C)CCCNC(=O)CCCCCCC/C=C\C[C@H](O)CCCCCC]

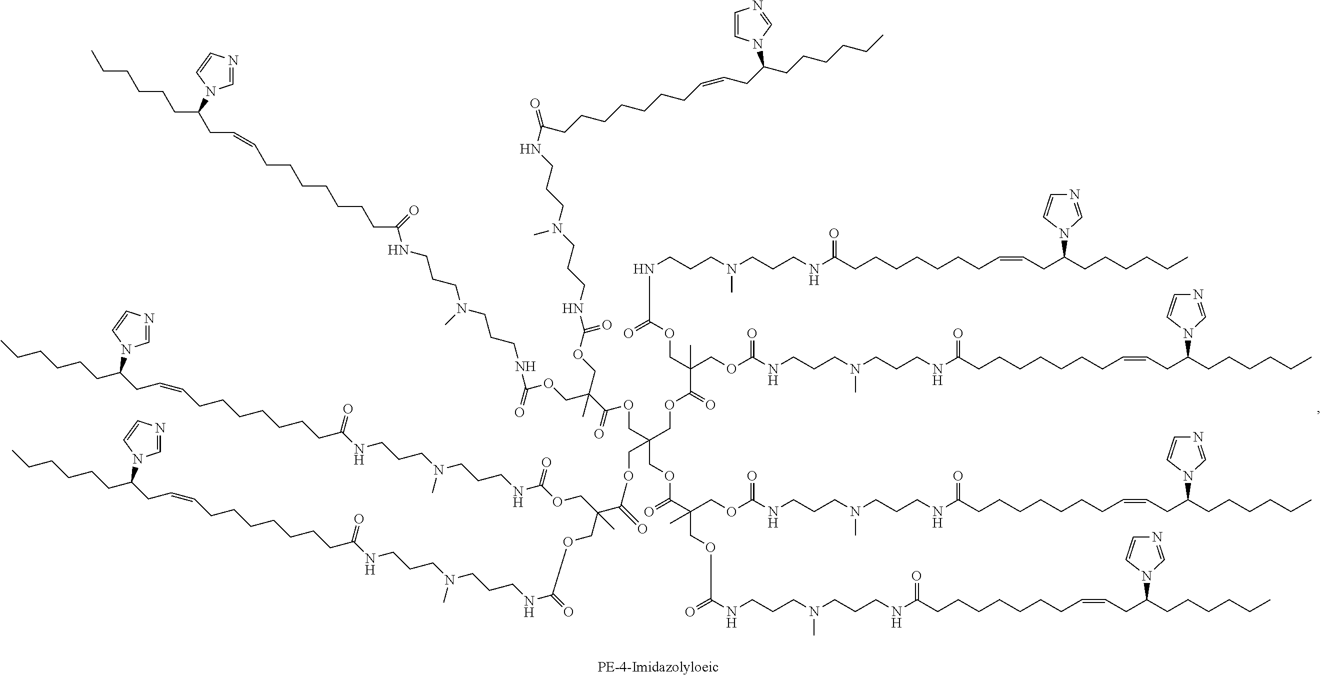

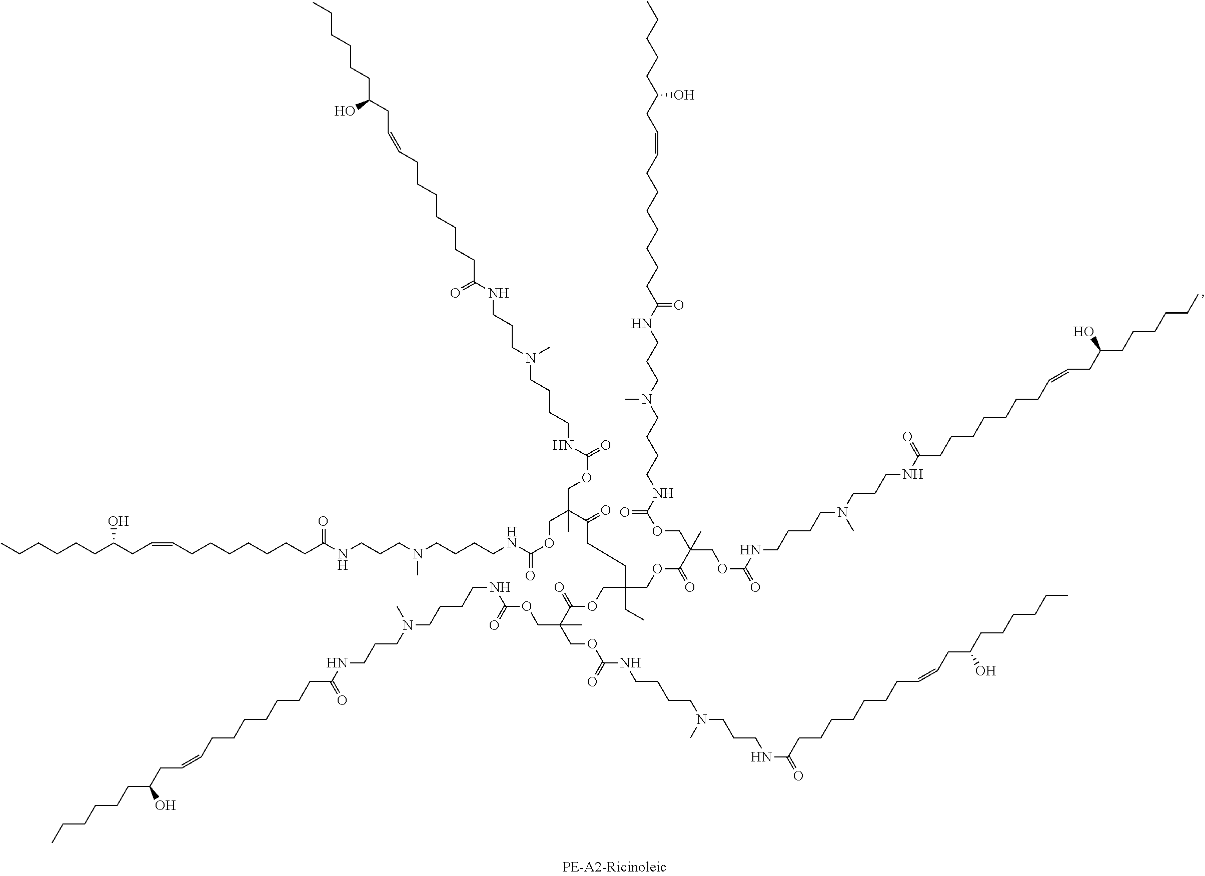 [SMILES: CCCCCC[C@H](O)C/C=C\CCCCCCCC(=O)NCCCN(C)CCCCNC(=O)OCC(C)(COC(=O)NCCCCN(C)CCCNC(=O)CCCCCCC/C=C\C[C@@H](O)CCCCCC)C(=O)CCC(CC)(COC(=O)C(C)(COC(=O)NCCCCN(C)CCCNC(=O)CCCCCCC/C=C\C[C@@H](O)CCCCC)COC(=O)NCCCCN(C)CCCNC(=O)CCCCCCC/C=C\C[C@@H](O)CCCCCC)COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC]

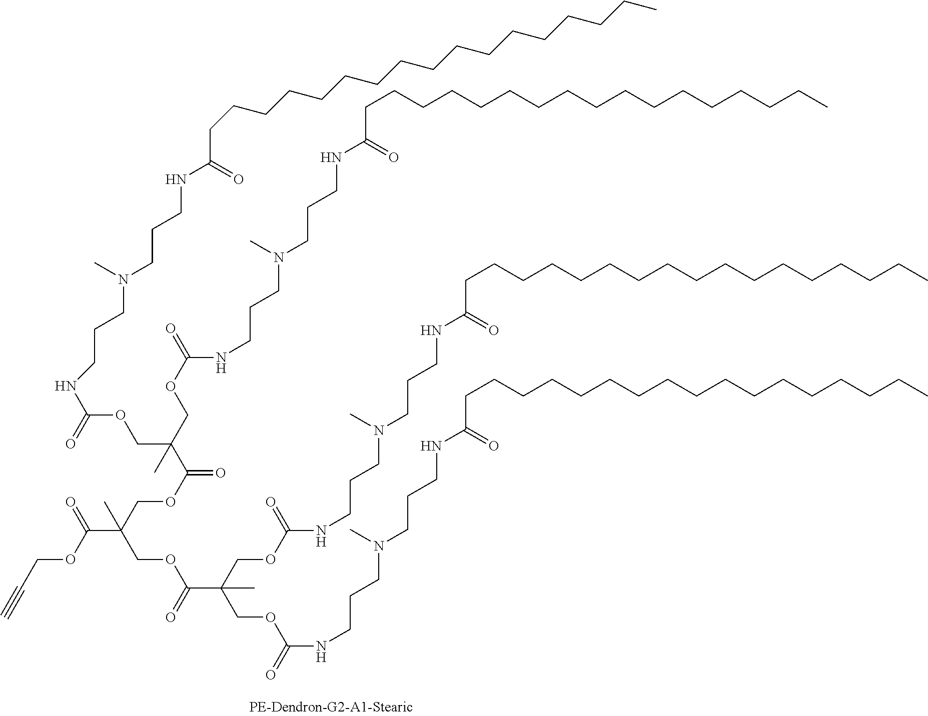 [SMILES: C#CCOC(=O)C(C)(COC(=O)C(C)(COC(=O)NCCCN(C)CCCNC(=O)CCCCCCCCCCCCCCCCC)COC(=O)NCCCN(C)CCCNC(=O)CCCCCCCCCCCCCCCCC)COC(=O)C(C)(COC(=O)NCCCN(C)CCCNC(=O)CCCCCCCCCCCCCCCCC)COC(=O)NCCCN(C)CCCNC(=O)CCCCCCCCCCCCCCCCC]

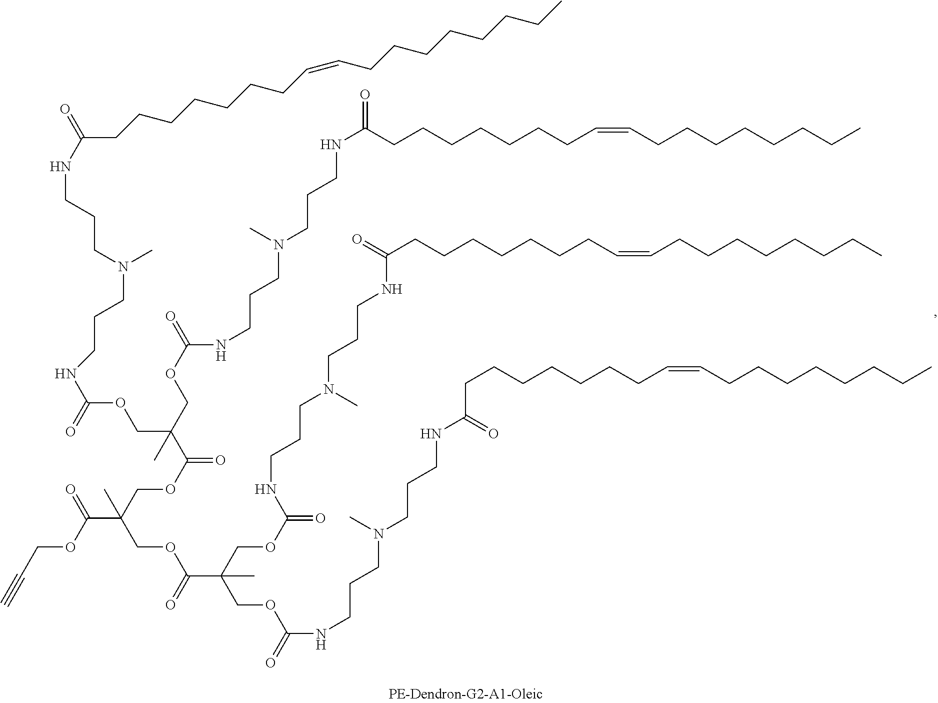 [SMILES: C#CCOC(=O)C(C)(COC(=O)C(C)(COC(=O)NCCCN(C)CCCNC(=O)CCCCCCC/C=C\CCCCCCCC)COC(=O)NCCCN(C)CCCNC(=O)CCCCCCC/C=C\CCCCCCCC)COC(=O)C(C)(COC(=O)NCCCN(C)CCCNC(=O)CCCCCCC/C=C\CCCCCCCC)COC(=O)NCCCN(C)CCCNC(=O)CCCCCCC/C=C\CCCCCCCC]

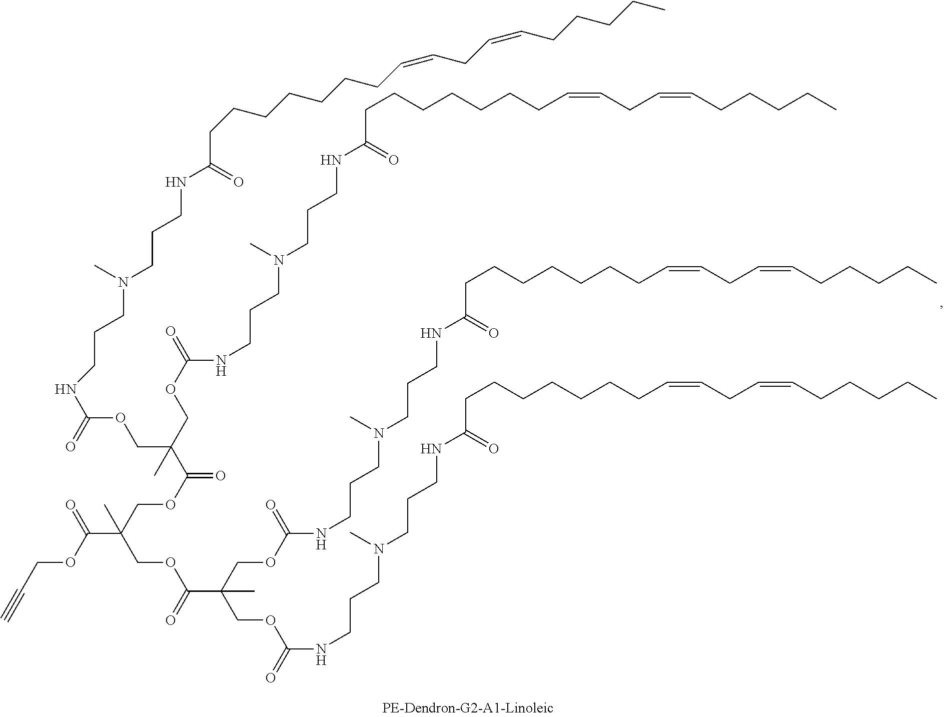 [SMILES: C#CCOC(=O)C(C)(COC(=O)C(C)(COC(=O)NCCCN(C)CCCNC(=O)CCCCCCC/C=C\C/C=C\CCCCC)COC(=O)NCCCN(C)CCCNC(=O)CCCCCCC/C=C\C/C=C\CCCCC)COC(=O)C(C)(COC(=O)NCCCN(C)CCCNC(=O)CCCCCCC/C=C\C/C=C\CCCCC)COC(=O)NCCCN(C)CCCNC(=O)CCCCCCC/C=C\C/C=C\CCCCC]

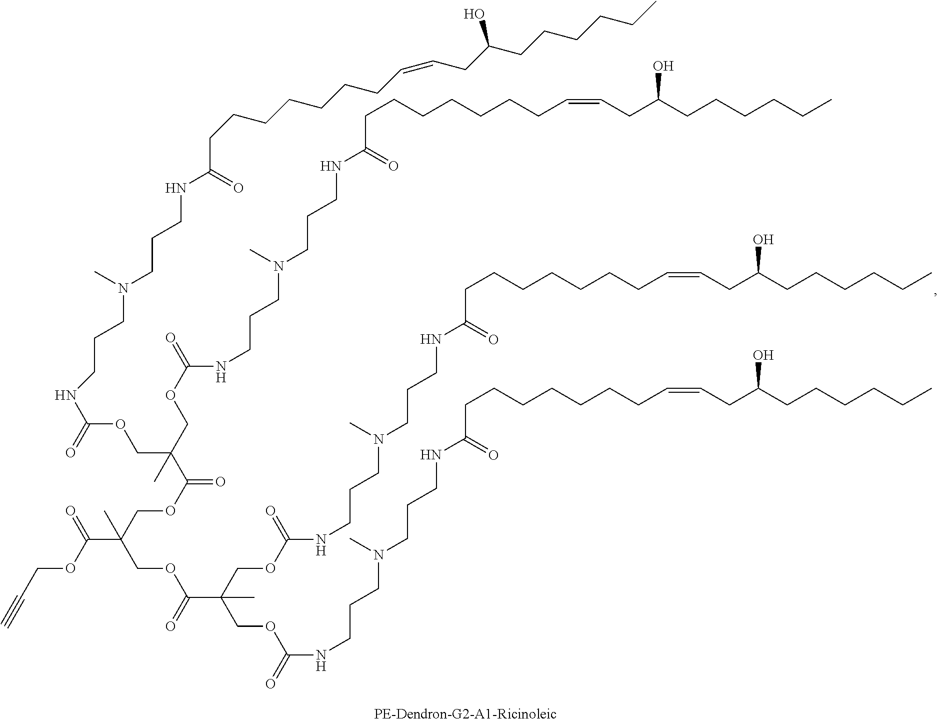 [SMILES: C#CCOC(=O)C(C)(COC(=O)C(C)(COC(=O)NCCCN(C)CCCNC(=O)CCCCCCC/C=C\C[C@@H](O)CCCCCC)COC(=O)NCCCN(C)CCCNC(=O)CCCCCCC/C=C\C[C@@H](O)CCCCCC)COC(=O)C(C)(COC(=O)NCCCN(C)CCCNC(=O)CCCCCCC/C=C\C[C@@H](O)CCCCCC)COC(=O)NCCCN(C)CCCNC(=O)CCCCCCC/C=C\C[C@@H](O)CCCCCC]

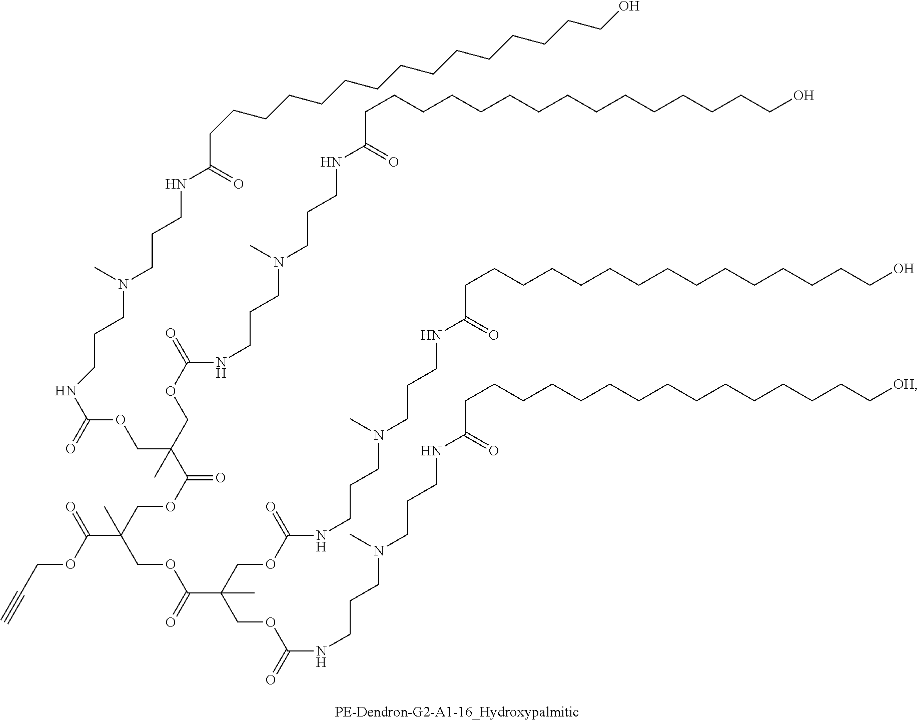 [SMILES: C#CCOC(=O)C(C)(COC(=O)C(C)(COC(=O)NCCCN(C)CCCNC(=O)CCCCCCCCCCCCCCCO)COC(=O)NCCCN(C)CCCNC(=O)CCCCCCCCCCCCCCCO)COC(=O)C(C)(COC(=O)NCCCN(C)CCCNC(=O)CCCCCCCCCCCCCCCO)COC(=O)NCCCN(C)CCCNC(=O)CCCCCCCCCCCCCCCO]

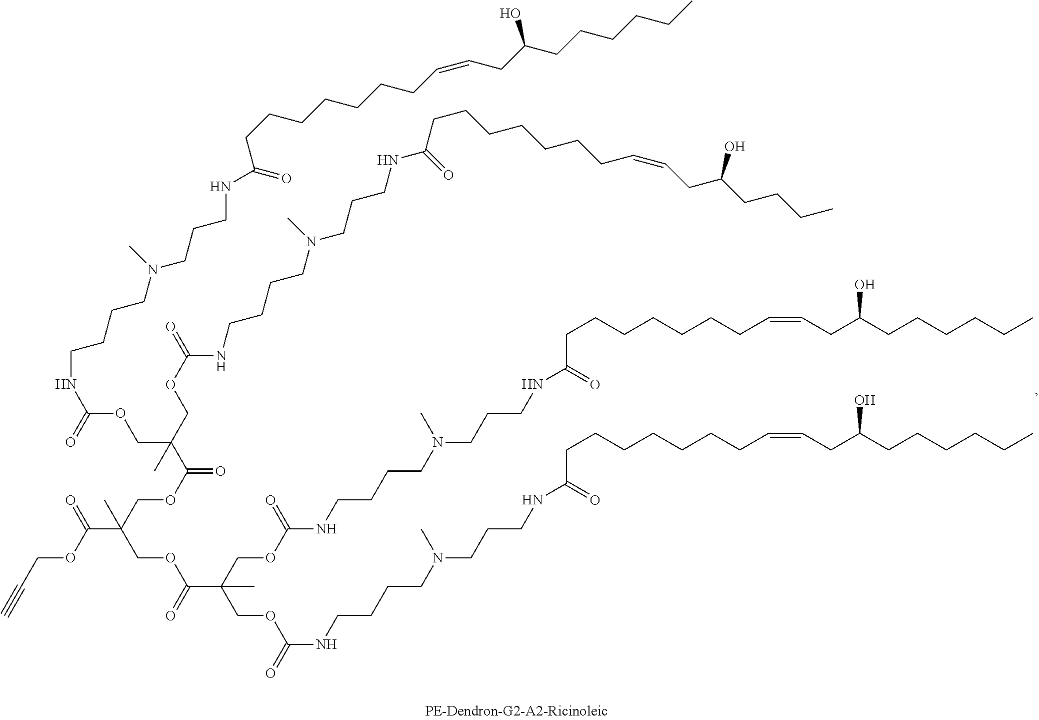 [SMILES: C#CCOC(=O)C(C)(COC(=O)C(C)(COC(=O)NCCCCN(C)CCCNC(=O)CCCCCCC/C=C\C[C@@H](O)CCCC)COC(=O)NCCCCN(C)CCCNC(=O)CCCCCCC/C=C\C[C@@H](O)CCCCCC)COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC]

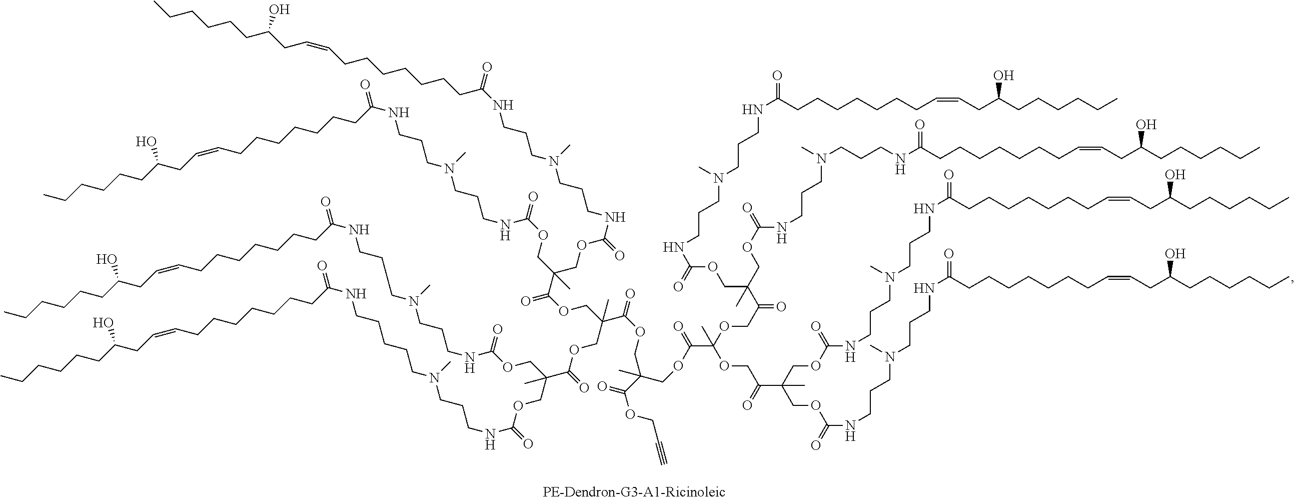

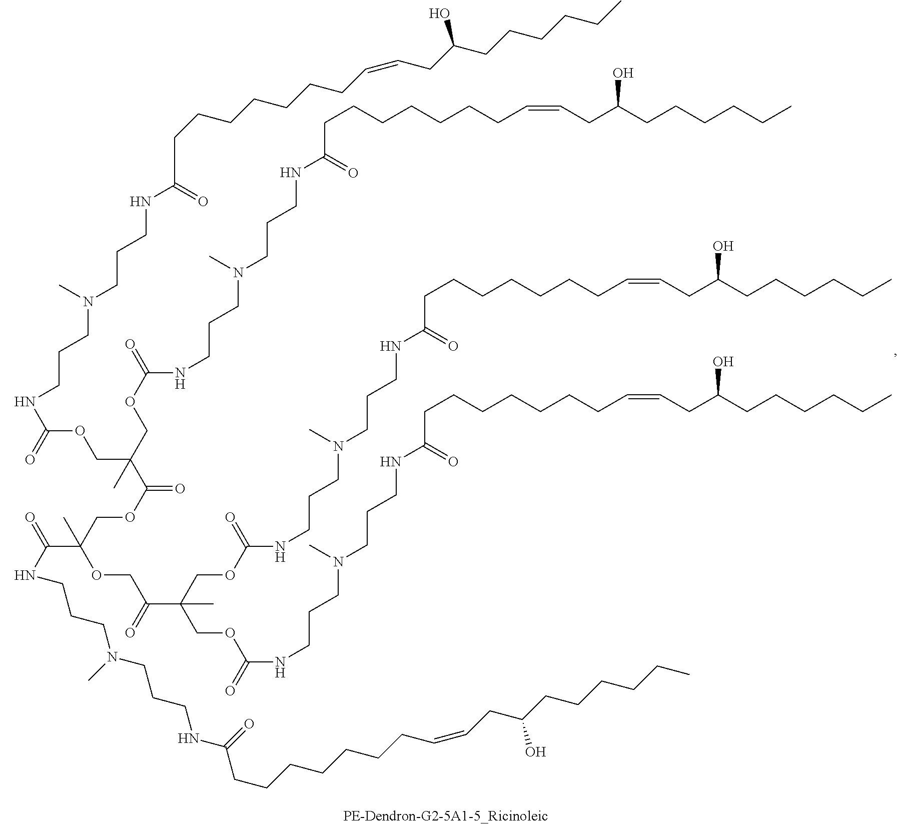

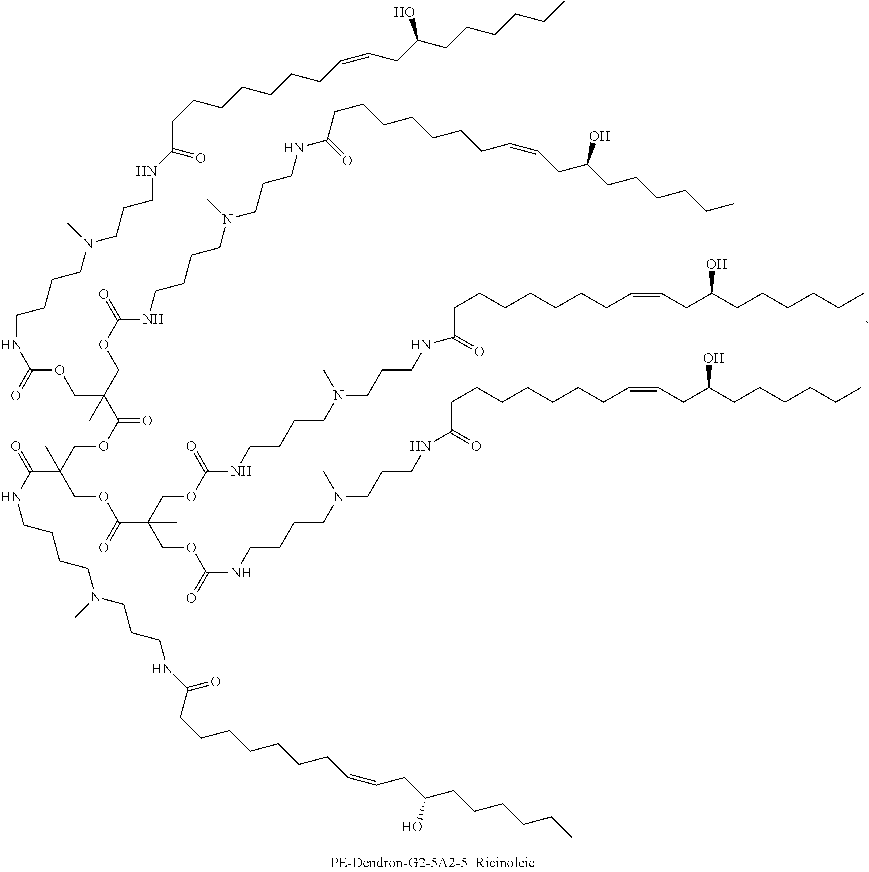

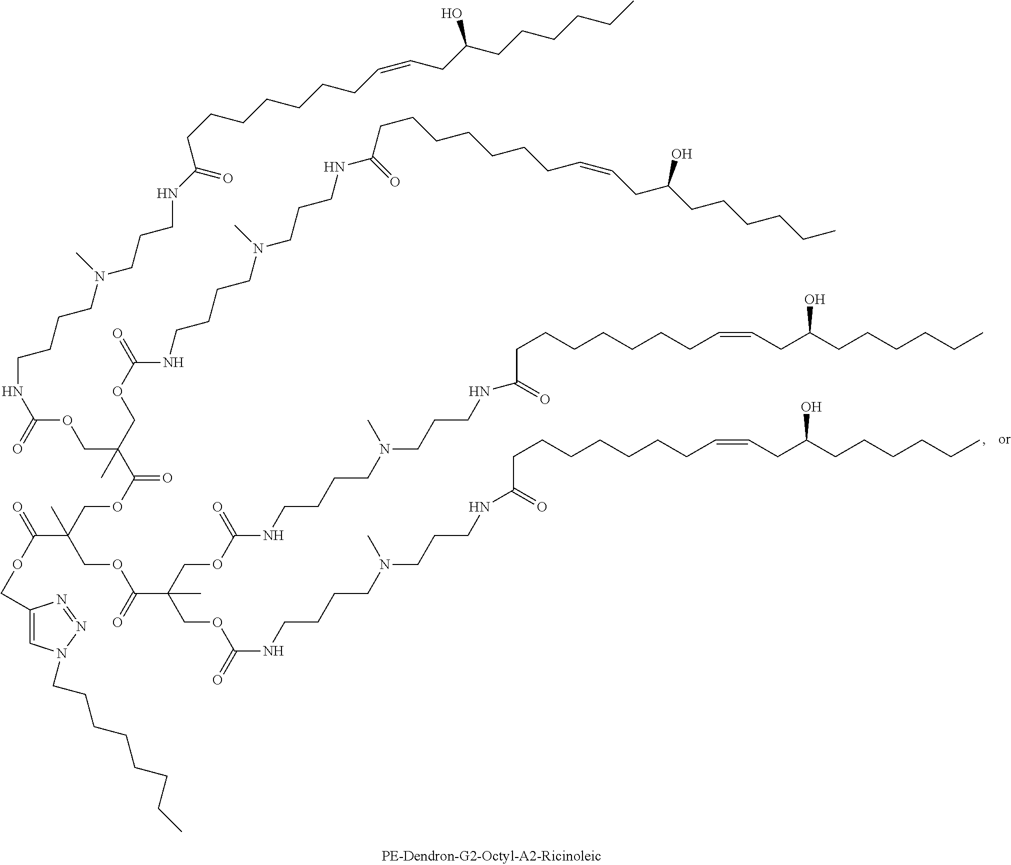 [SMILES: CCCCCCCCn1cc(COC(=O)C(C)(COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC)COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC)nn1]

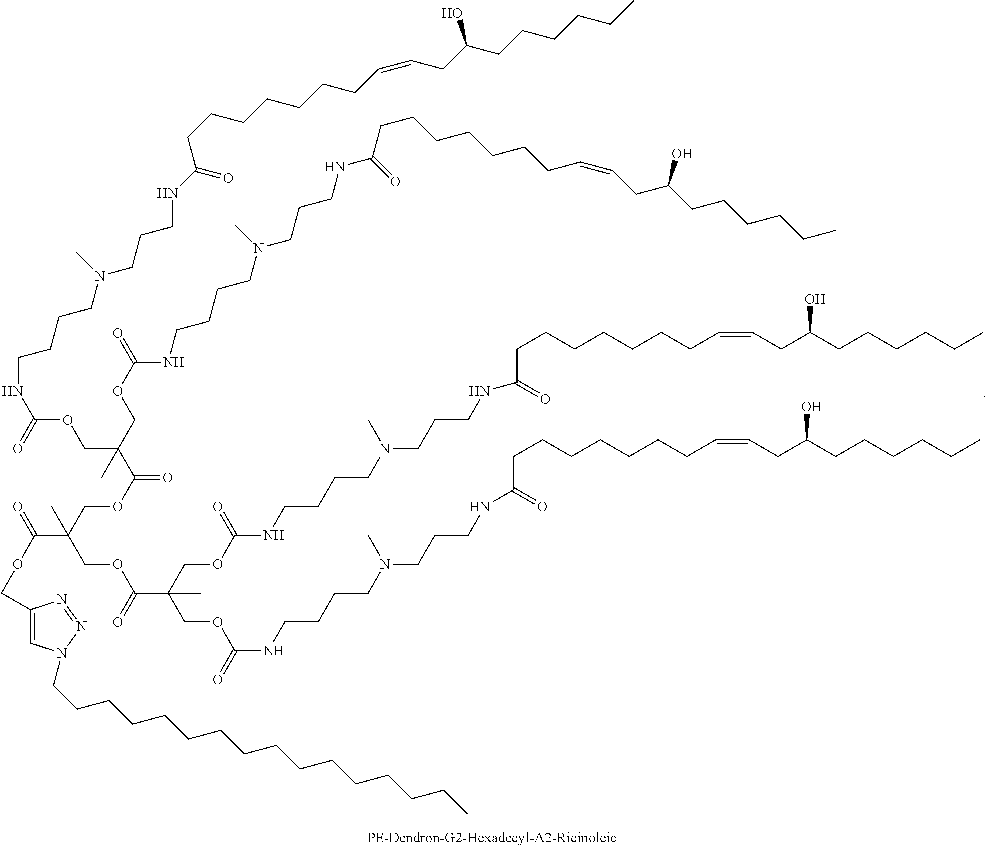 [SMILES: CCCCCCCCCCCCCCCCn1cc(COC(=O)C(C)(COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC)COC(=O)C(C)(COC(=O)NCCCCN(C)CCCNC(=O)CCCCCCC/C=C\C[C@@H](O)CCCCCC)COC(=O)NCCCCN(C)CCCNC(=O)CCCCCCC/C=C\C[C@@H](O)CCCCCC)nn1]

### Example 3. Nanoparticle Formulation

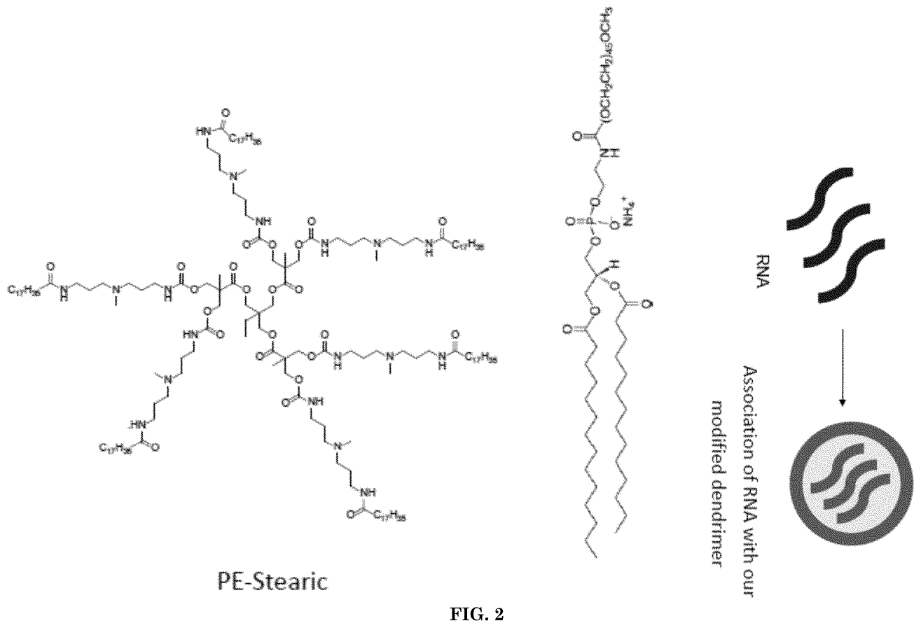

FIG. 2 illustrates a process for preparing a nanoparticle composition designed for improved self-assembly. Nanoparticles were formulated by direct mixture of 30 μl of an ethanol phase containing modified dendrimer (PE-Stearic) in combination with 1,2-dimyristoyl-sn-glycero-3-phosphoethanolamine-N-[methoxy(polyethyleneglycol)-2000] (PEG-lipid, Avanti Polar Lipids) with 90 μl of Luciferase mRNA diluted with ultraPure, DNase/RNase-free, endotoxin-free distilled water (Invitrogen) and sterile 100 mM (pH 5.0) QB Citrate Buffer (Teknova) to a final citrate concentration of 10 mM. The resulting nanoparticles contained an 6:1:2 mass ratio of modified dendrimer to PEG-lipid to RNA. Formulations were diluted 1000-fold for analysis of particle size distribution, Z-average, and derived count rate using a Zetasizer Nano ZS (Malvern Panalytical).

### Example 4. Hydrodynamic Size Measurement

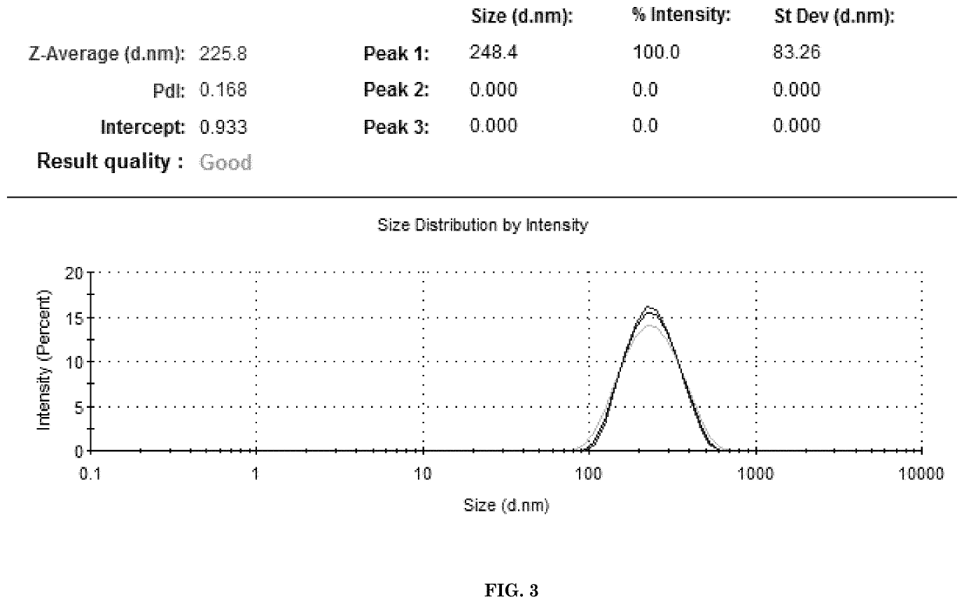

FIG. 3 illustrates distribution of the nanoparticle compositions measured as the intensity based on size (d.nm; diameter in nm) of the nanoparticles. Referring to FIG. 3, the “Z average” is the intensity weighted mean hydrodynamic size of the ensemble collection of particles measured by dynamic light scattering (DLS). Referring to FIG. 3, the strongest intensity was observed for the nanoparticles of 248.4 d.nm in size.

### Example 5. Gel Retardation Assays

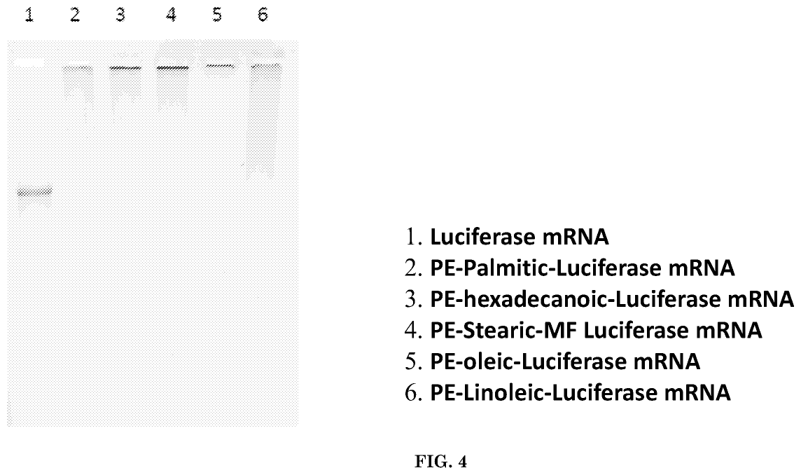

Agarose gel electrophoresis was performed to evaluate the binding of modified dendrimer with RNA according to the known method (Geall et al. 10.1073/pnas.1209367109, which is incorporated herein by reference as if fully set forth. FIG. 4 is a photograph of the agarose gel showing the binding of the modified dendrimer with RNA. The gels were stained with ethidium bromide (EB) and gel images were taken on a Syngene G Box imaging system (Syngene, USA). Referring to FIG. 4, lane 1 contained the unformulated Luciferase mRNA, lane 2 contained the product of formulation of the PE-palmitic dendrimer and Luciferase mRNA, lane 3 contained the product of formulation of the PE-heptadecanoic and Luciferase mRNA, lane 4 contained the product of formulation of the PE-stearic and Luciferase mRNA, lane 5 contained the product of formulation of the PE-oleic and Luciferase mRNA, lane 6 contained the product of formulation of the PE-linoleic and Luciferase mRNA. Before loading, the samples were incubated with formaldehyde loading dye, denatured for 10 min at 65° C. and cooled to room temperature. The gel was run at 90 V and gel images were taken on a Syngene G Box imaging system (Syngene, USA). For RNA detection, the gel was stained with ethidium bromide. Referring to FIG. 4, the lower band corresponds to the small size free RNA (lane 1) and the top bands represent the large size nanoparticles formed by binding of the RNA to the dendrimer carriers.

### Example 6. Material Stability

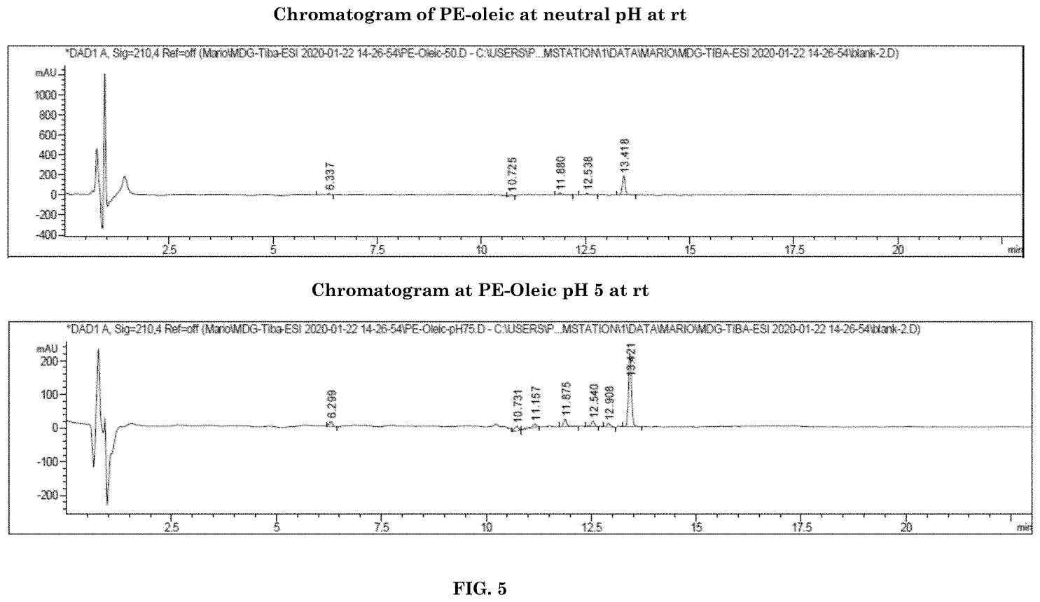

Polyester dendrimers constitute unique class of materials because they degrade easily because of the hydrolytic susceptibility of the ester bonds. However an ideal nucleic acid carrier should be stable at the formulation pH as well as the storage pH. Therefore, the stability of the dendrimer materials was checked in different pH. FIG. 5 shows the LCMS chromatograms of dendrimers at neutral pH and pH 5. The chromatogram showing single peak (elution time 13.42 min) at both neutral pH and pH 5 at room temperature indicates that the polyester dendrimers structures should be intact while formulating RNA at pH 5.

### Example 7. Colloidal Stability of Nanoparticle Compositions Containing Polyester Dendrimers Modified with Fatty Acids

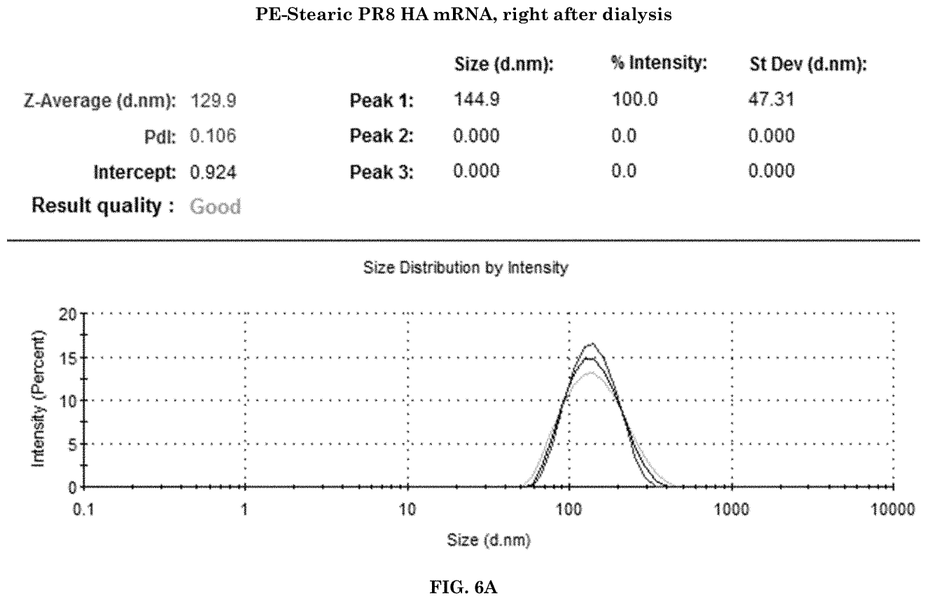

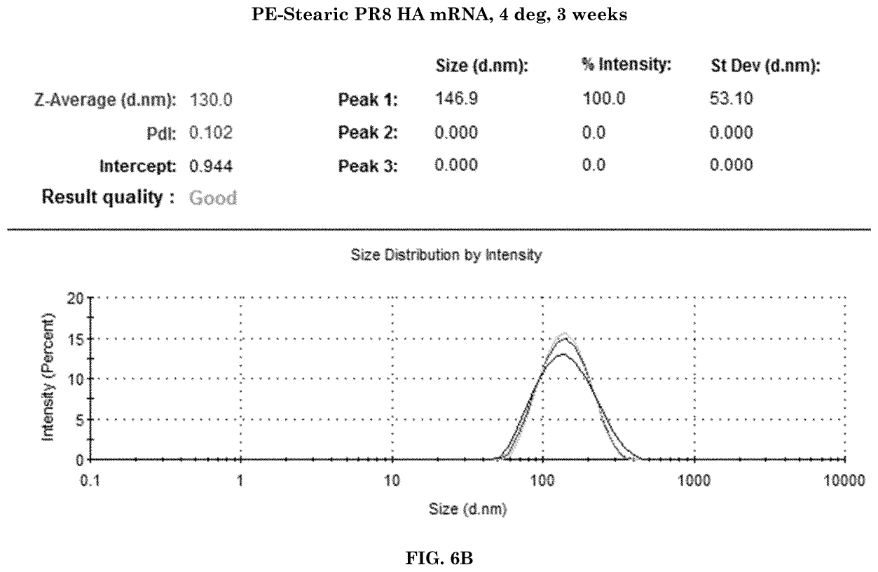

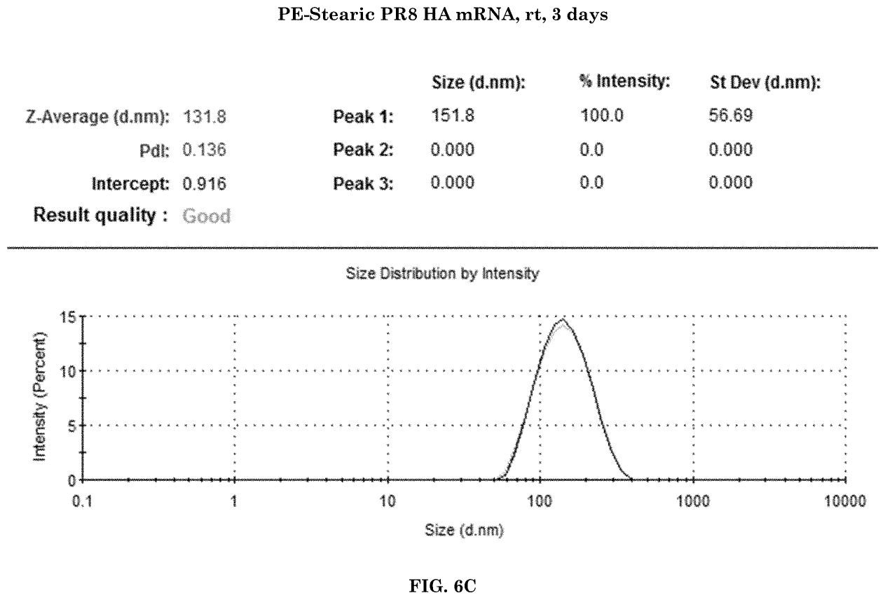

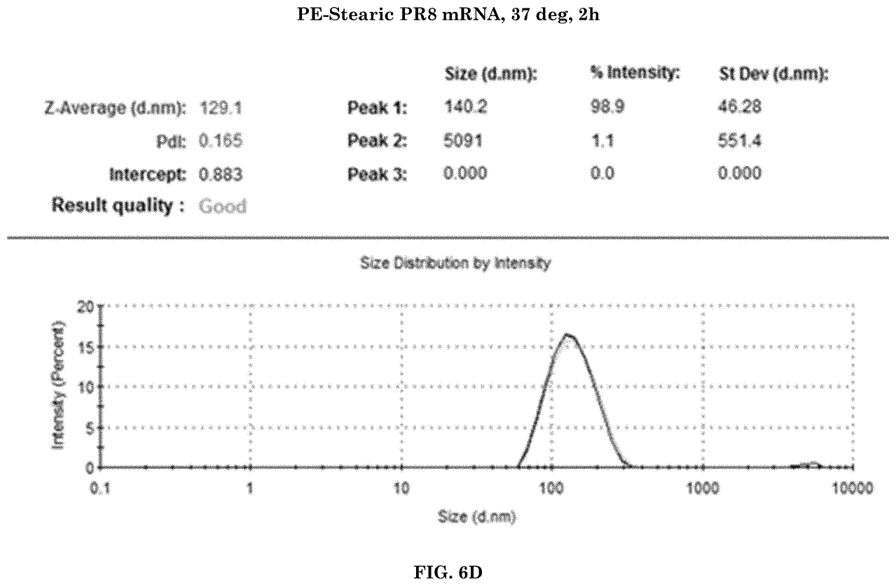

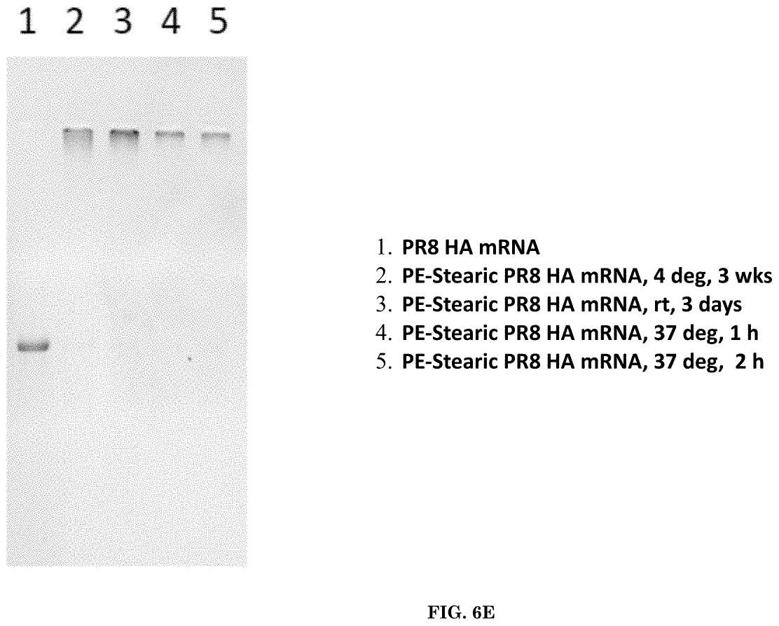

The stability of the nanoparticles was determined by comparing the particle size distribution 21 days after formulation and storage at 4° C. with the particle size distribution measured immediately after the 2 hrs dialysis. The same polyester dendrimer displayed good stability in PBS at room temperature as well as at 37° C. without sufficient aggregation. FIGS. 6A-6E show the stability of RNA-PE stearic nanoparticles in PBS as measured by DLS and by agarose gel electrophoresis. FIG. 6A illustrates the distribution of the nanoparticle compositions after dialysis measured as the intensity based on size (d) of the nanoparticles. FIG. 6B shows the stability of PE-Stearic PR8 HA mRNA as measured by DLS following storage for 3 weeks at 4° C. FIG. 6C shows the stability of PE-Stearic PR8 HA mRNA as measured by DLS following storage for 3 days at room temperature (RT). FIG. 6D shows the stability of PE-Stearic PR8 HA mRNA as measured by DLS following storage for 2 hours at 37° C. FIG. 6E shows the stability results based on the gel retention assay: lane 1-PR8 HA mRNA, lane 2—PE-Stearic PR8 HA mRNA, 4 deg, 3 weeks, lane 3-PE-Stearic PR8 HA mRNA, rt, 3 days, lane 4—PE-Stearic PR8 HA mRNA, 37° C., 1 hour, and lane 5—PE-Stearic PR8 HA mRNA, 37° C., 2 hours. It was observed that the particle size distributions were the same, indicating stability under these storage conditions.

### Example 8. Cell-Based Luciferase mRNA Expression Analysis

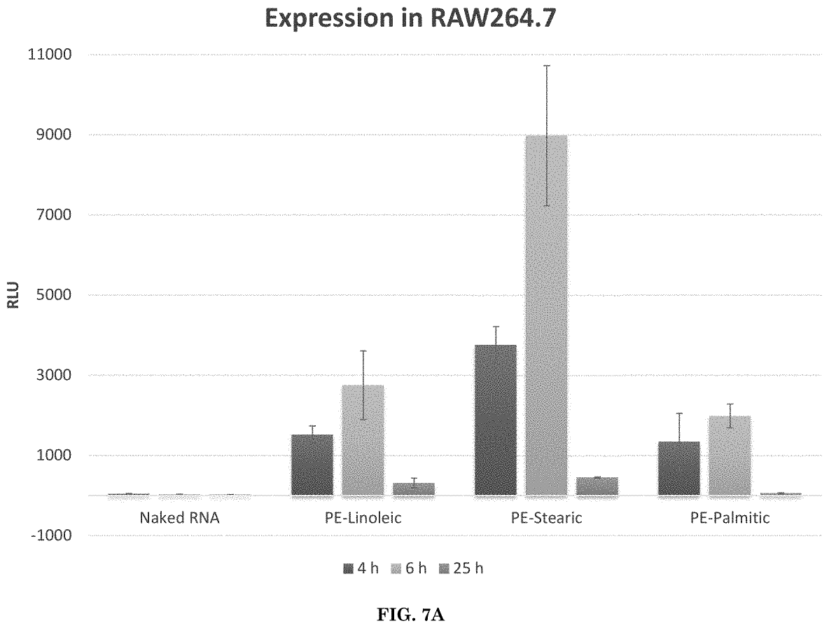

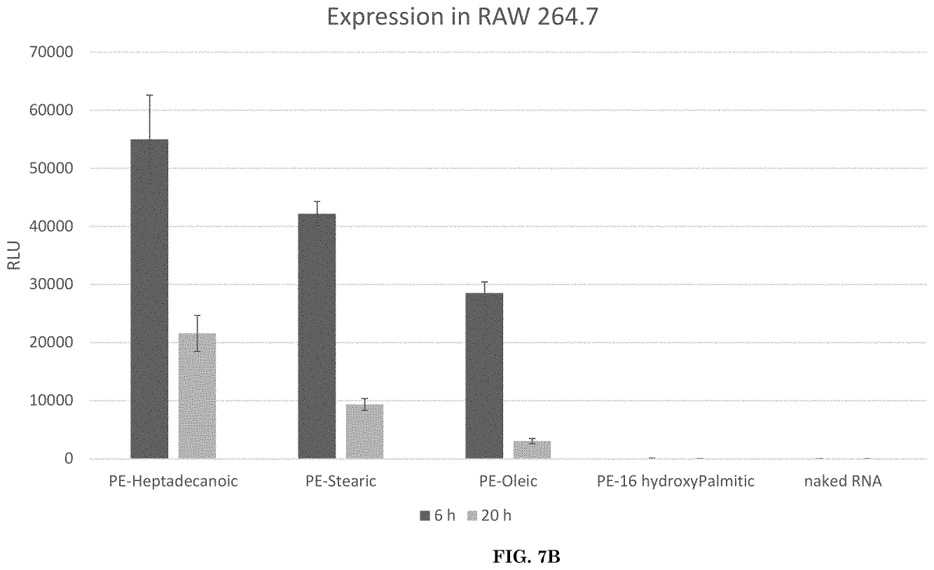

A luciferase cDNA expression cassette was designed consisting of a 5′UTR, luciferase ORF, a 3′UTR, and polyA tail. This cassette was synthesized and cloned into the pcDNA3.1 vector at NheI/KpnI sites downstream of the T7 promoter by GenScript. The derived expression vector was used as the DNA template for synthesis of luciferase mRNA by in vitro T7-based transcription (Hongene, #ON-040) and vaccinia capping system (Hongene, #ON-028). Following purification by lithium chloride precipitation, the luciferase mRNAs was formulated with polyester dendrimers to form nanoparticles for further analysis. Acquired from ATCC, the RAW264.7 (ATCC, TIB-71) and A549 (ATCC, CCL-185) cells were grown and maintained according to the protocols suggested by ATCC. To test newly formulated nanoparticles for their ability to deliver mRNA into mammalian cells resulting in protein expression, monolayers of RAW264.7 or A549 cells grown in 96-well plate were transfected with 50 ng of luciferase mRNA nanoparticle per well in 50 μl of PBS. Following incubation at 37° C. for 1 hour, growth medium was added at 50 μl per well. At designated time points post-transfection, cells were lysed and luciferase activity was measured using luciferase one-step glow assay kit (ThermoFisher, #88263). FIGS. 7A and 7B show luciferase expression in cell culture from luciferase mRNA delivered into mammalian cells in modified polyester dendrimer-based nanoparticles and measured by quantification of intracellular luciferase activity using a luminescence assay. FIG. 7A illustrates cell culture expression of luciferase mRNA delivered in nanoparticles containing PE-linoleic, PE-stearic, PE-palmitic compared to the naked luciferase mRNA. FIG. 7B illustrates cell culture expression of luciferase mRNA delivered in nanoparticles containing PE-heptadecanoic, PE-stearic, PE-oleic, and PE-16-hydroxypalmitic compared to the naked luciferase mRNA. Referring to these figures, it was observed that all the RNA nanoparticles were able to be taken up by RAW264.7 cells leading to gene expression

### Example 9. Western Blot Analysis of HA Expression

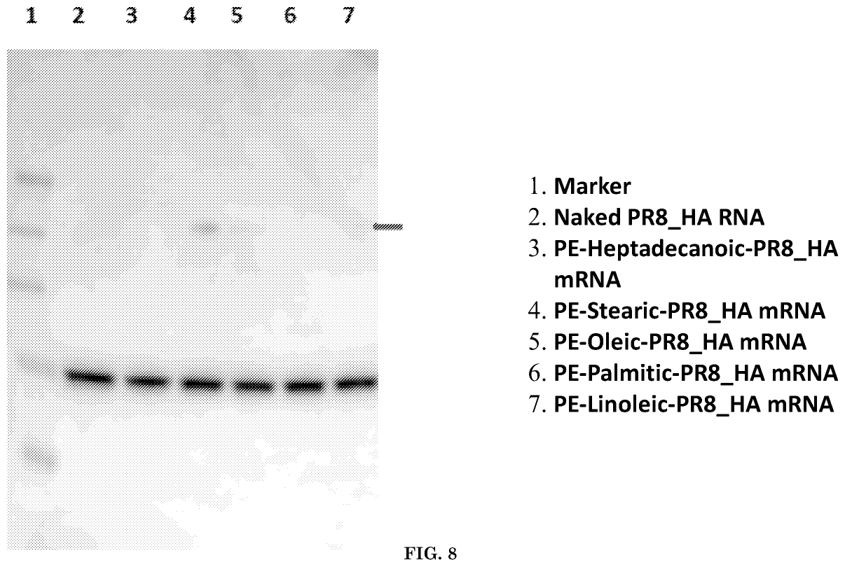

The influenza HA expression cassette consisting of the full-length HA ORF (PR8 HA) flanked by a 5′UTR and a 3′UTR followed by a polyA tail, was synthesized and cloned into the pcDNA3.1 vector downstream of the T7 promoter. The HA expression vector was used as the DNA template for synthesis of HA mRNA by in vitro T7-based transcription (Hongene, #ON-040) and vaccinia capping system (Hongene, #ON-028). Following purification by lithium chloride precipitation, the HA mRNA was formulated to form dendrimer-based nanoparticles. For western blot analysis, A549 cells grown in 12-well plate were transfected with 2 μg of HA mRNA nanoparticle per well in 1 ml of PBS. Following incubation at 37° C. for 1 hour, 1 ml of growth medium was added to each well. At 24 h post-transfection, cells were collected and lysed with RIPA buffer (Biovision, #2114) according to manufacturer's protocol. The cell lysate was mixed with Laemmli sample buffer, analysed by SDS-PAGE (10% Bis-Tris Plus Gels, ThermoFisher, #NW00100BOX) and electroblotted onto PVDF transfer membranes (ThermoFisher, #IB24002). After blocking in 5% non-fat dry milk and 0.1% Tween-20 in PBS overnight, membranes were incubated with Influenza A virus H1N1 HA antibody (GeneTex, #GTX127357) diluted in the same buffer for 1 h at room temperature. FIG. 8 illustrates the Western blot analysis of HA expression. The HA protein (FIG. 8, shown by an arrow) was visualized on a Chemi-XRS system (SynGene) using sheep anti-rabbit IgG (H+L) secondary antibody conjugated to HRP (ThermoFisher, #A12172) and a chemiluminescence detection system (ProSignal® Femto ECL Reagent, Prometheus #20-302). Referring to FIG. 8, it was observed that some of the RNA nanoparticles were able to be taken up by A549 cells leading to gene expression.

### Example 10. Hemagglutinin Inhibition Assay (HAI)

HAI tests were performed on vaccinated animal sera according to the standard recommended WHO protocol [World Health Organization. (2002). WHO manual on animal influenza diagnosis and surveillance 2002.5 Rev. 1. 2002]. Sera were treated with receptor destroying enzyme (Denka Seiken Co. Ltd, Tokyo, Japan) at 37° C. overnight then heat inactivated for 30 minutes at 56° C. After cooling down to RT, those serum samples were mixed with 2 volume of 25% suspension of turkey red blood cells (RBC, Rockland Immunochemicals) and incubated at room temperature for 2 hours followed by centrifugation to remove RBC. The treated sera, now considered to be at a 1:10 dilution, were serially diluted and incubated with 4 HA units of inactivated Influenza A virus (PR/8/34, Virusys #IAV210) for 30 minutes at room temperature. An equal volume of 0.5% turkey RBC was added to each well and incubated for 30 minutes at room temperature. The HAI titer was read as the highest dilution of serum that completely inhibited hemagglutination.

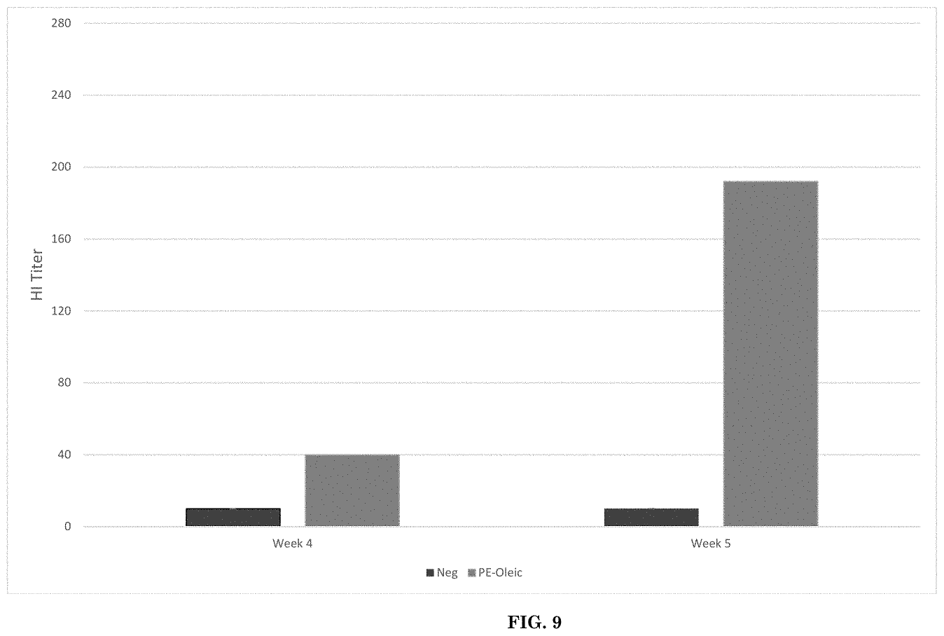

After prime vaccination, humoral immune responses as measured by HI titers were observed in HA mRNA group immunized with the 5 μg of nanoparticle vaccine formulated using PE-Oleic (FIG. 9). At week 4, the average HI titer in this group was 40 and continued to rise to an average of 192 at week 5, one week after boost immunization with the same vaccine dosage. This is in contrast to the group immunized with naked mRNA where the HI titer remained at base line of 10 at all time points.

### Example 11. Nanoparticle Composition Containing DNA

SEAP DNA was produced by cloning the SEAP sequence into pcDNA3 plasmid. This plasmid contains the necessary origin of replication and ampicillin resistance genes necessary for maintenance and propagation in bacterial culture, the mammalian CMV promoter upstream of the gene cloning site to drive expression in mammalian cells in tissue culture, and the bacteriophage T7 transcription promoter downstream of the CMV promoter to allow in vitro transcription of mRNA encoding the cloned genetic sequence that terminates with a BspQI restriction site. The In-Fusion (Clontech Laboratories) cloning kit was used to construct the plasmid from commercially-sourced DNA fragments.

PE-Linoleic-SEAP DNA Nanoparticles were formulated by direct mixture of 20 μl of an ethanol phase containing modified dendrimer (PE-Linoleic) in combination with 1,2-dimyristoyl-sn-glycero-3-phosphoethanolamine-N-[methoxy(polyethylene glycol)-2000] (PEG-lipid, Avanti Polar Lipids) with 60 μL of SEAP DNA diluted with ultrapure, DNAase/RNase-free, endotoxin-free distilled water (Invitrogen) and sterile 100 mM (pH 5.0) QB Citrate Buffer (Teknova) to a final citrate concentration of 10 mM, and a final DNA concentration of 0.38 mg/mL. The ethanol and citrate streams were mixed at a 1:3 ethanol volume to citrate volume ratio to produce nanoparticles. The resulting nanoparticles contained an 6:1:2 mass ratio of modified dendrimer to PEG-lipid to DNA. Formulations were diluted 1000-fold for analysis of particle size distribution, Z-average, and derived count rate using a Zetasizer Nano ZS (Malvern Panalytical).

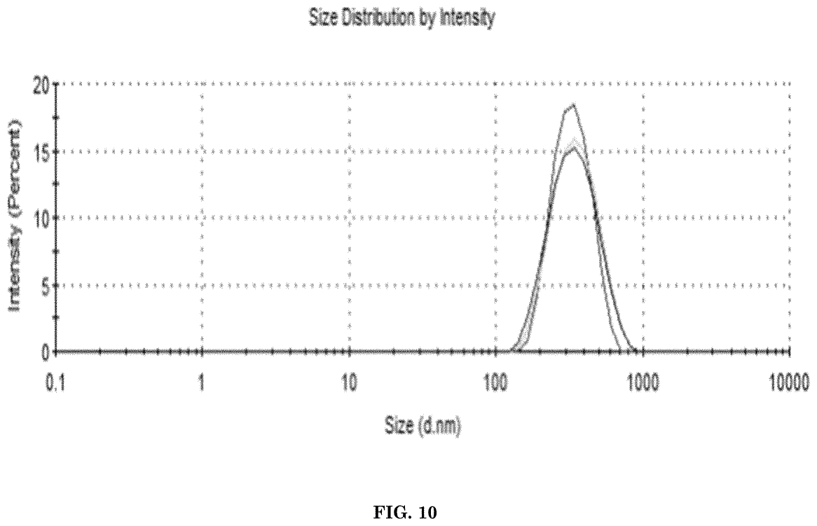

FIG. 10 illustrates particle size distribution of nanoparticles generated by mixture of PE-Linoleic modified dendrimer and SEAP DNA.

TABLE 1
DLS analysis of nanoparticles containing
modified dendrimer and SEAP DNA

| | | Polydispersity | | Derived |
|---|---|---|---|---|
| Sample | Z-Ave | Index | | Count Rate |
| Name | (d · nm) | (PdI) | Y-Intercept | (kcps) |
| PE-Linoleic | 324.4 | 0.130 | 0.821 | 206.6 |

To test the ability of these nanoparticles to express SEAP in vitro, 293T cells were treated with nanoparticles. Each well of a 96 well dish of 293 Ts was treated with 10 μL (approximately 0.25 μg) of each formulation product diluted into a final volume of 200 μL with a 1:1 Optimem:PBS mix. The nontreated wells had 200 μL of 50/50 PBS/OptiMEM. Twenty four hours post-treatment or transfection, conditioned media from each culture was collected. Media was analyzed using the QuantiBlue assay (InvivoGen). 180 μL QuantiBlue reagent was combined with 50 μL of media from wells. Readings were taken after 40 minutes by measuring absorbance at 650 nm as directed by the manufacturer's protocol. For the no treatment Negative Control, 50 μL of media from a well not treated with nanoparticles was added to 180 μL QuantiBlue Reagent. These samples were treated the same way as all other samples in all steps of the assay.

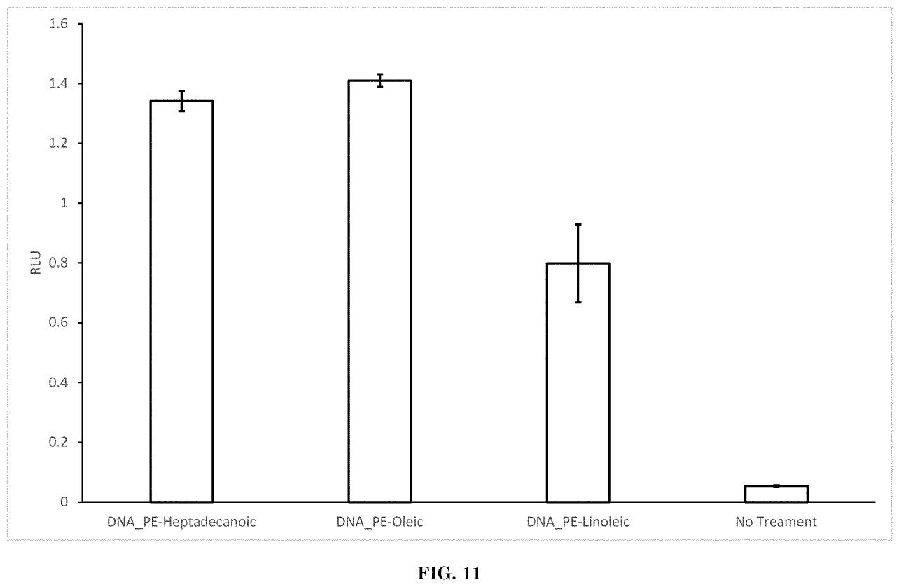

FIG. 11 illustrates in vitro SEAP expression of nanoparticle formulations with modified dendrimers used to treat 293T cells. SEAP DNA formulated with PE-Heptadecanoic, PE-Oleic and PE-Linoleic produced nanoparticles that resulted in SEAP expression.

### Example 12. Nanoparticle Compositions Containing Polyester Dendrons Modified with Fatty Acids

Nanoparticles containing the PE Dendron modified to include fatty acid tails in its terminal (e.g PE Dendron_G2-A1-Ricinoleic):DSPC:cholesterol:DMG-PEG2k at molar ratios of 1:0.5:2.4:0.035 were formulated using NanoAssemblr Benchtop (Precision NanoSystems Inc, Vancouver, BC, Canada)). RNA was diluted with DNase/RNase-Free, endotoxin free distilled water and sterile citrate buffer to a final desired pH. Total flow rate was maintained at 12 mL per min at a 3:1 ratio of aqueous to organic phase for formulating on the Benchtop. Using glassware washed for 24 hours in 1.0 M NaOH for endotoxin removal and sterilized in a steam autoclave, or depyrogenated by heating at 250° C. for 24 hour, nanoparticles were dialyzed against sterile, endotoxin-free PBS using 20,000 molecular weight cutoff dialysis. Dialyzed nanoparticles were sterile filtered using 0.2 micron poly(ether sulfone) filters and characterized with a Zetasizer NanoZS machine (Malvern). The size distributions were characterized by a single peak with a low polydispersity index, indicating a relatively monodisperse size. Encapsulation efficiency was measured to be 95% for the nanoparticle composition containing PE Dendron_G2-A1-Ricinoleic and SEAP Replicon RNA (formulated at pH 5) using Ribogreen® assay (Geall et al. 10.1073/pnas.1209367109 which is incorporated herein by reference as if fully set forth).

To test formulations of modified PE Dendron, the secreted embryonic alkaline phosphatase SEAP reporter system was used. For in vivo tests, mice were injected with nanoparticles at a dose of 5 ug of SEAP RepliconRNA, and 16 hrs later, serum was collected from the mice. The amount of quantified using the Invitrogen NovaBright™ Phospha-Light™ EXP Assay kits for SEAP detection according to the manufacturer's protocol. The amount of SEAP in the mouse serum samples are reported in Arbitrary Units (A.U.) as measured in a BioTek Synergy HTX microplate reader. Error bars are ±S.E.M. Referring to FIG. 12, it was observed that the SEAP amount was higher with the pH 5.0 formulation, as compared to pH 4.0 because the RNA was released sooner from the pH 5.0 formulation due to weaker binding.

### Example 13. Western Blot Analysis of Spike Expression

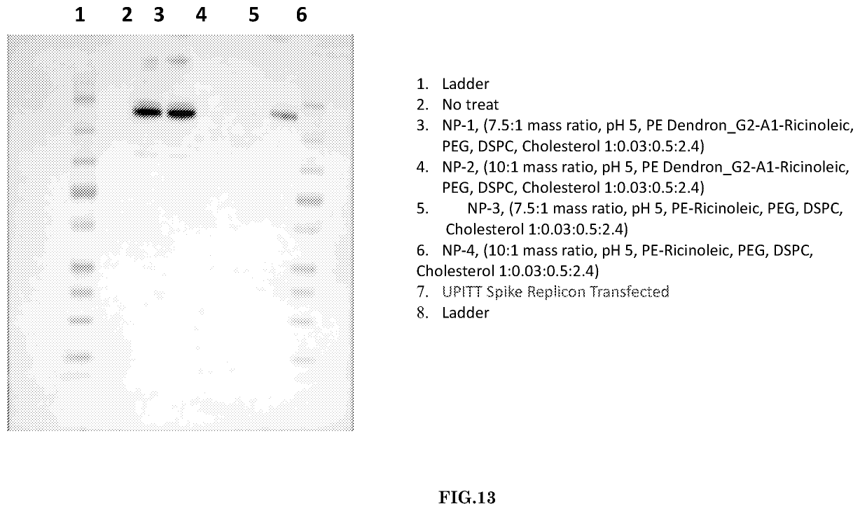

BHK cells at ˜80% confluency in 12 well dishes were treated with Spike Replicon RNA formulated with Polyester dendron and dendrimer molecules using the method described in Example 12. Media was removed and the cells were treated with 5 μg of nanoparticles. Cells were incubated at 37° C. overnight, and harvested ˜16 hours post treatment by scraping. Cell pellets were centrifuged at 13000 rpm for 3 minutes, and resuspended in 100 uL RIPA (supplemented with HALT™ protease and phosphatase inhibitor cocktail and Pierce™ Universal Nuclease), followed by the addition of 25 μL 6×SDS Laemmli buffer. Samples were boiled for 10 minutes and centrifuged to remove particulates. 20 μL of each sample was separated by electrophoresis on a 10-well Bolt Bis-Tris SDS-PAGE gel. A sample from an unrelated experiment that had previously been validated for Spike expression was included as a positive control. Protein was transferred from the gel to a PVDF membrane using an iBlot2 dry transfer system. After transfer, the membrane was blocked with TBST+10% milk for 30 minutes prior to the addition of Rabbit-anti-Spike antibody at a 1:1000 dilution in TBST+10% milk. The membrane was incubated at room temperature for 45 minutes, followed by three washes with TBST. The membrane was then incubated in TBST+10% milk with sheep anti-rabbit HRP antibody at a 1:2000 dilution for 30 minutes. The membrane was washed three times with TBST and then developed using Prometheus™ ProSignal™ Dura chemiluminescent substrate. The chemiluminescent reaction was visualized using a GeneSys Imaging system. Referring to FIG. 13, it was observed that RNA nanoparticles formulated using PE Dendron_G2-A1-Ricinoleic were able to be taken up by BHK cells leading to gene expression.

### Example 14. COVID-19 Spike Trimer Direct Serum ELISA

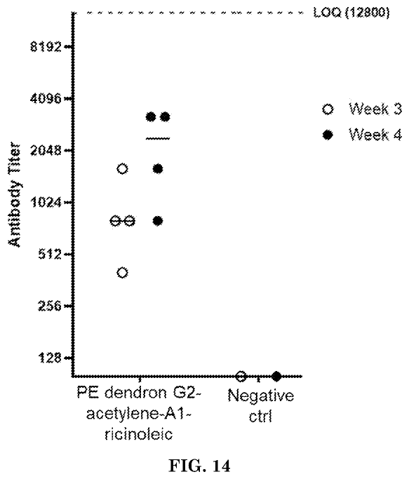

Mice (BALB/c) were vaccinated by IM injection bilaterally in the leg muscle with 10 μg of Spike Replicon RNA formulated with PE Dendron-G2-Ricinoleic using the method described in Example 12. (in a total volume of 100 μL PBS). Mice were bled both 21 days and 28 days post injection, and serum was isolated from whole blood by centrifuging coagulated samples at 10000 RCF for 1.5 minutes. This serum was assayed for anti-Spike antibody titer by direct ELISA. Nunc MaxiSorp ELISA plates were coated overnight with pH 9.5 bicarbonate coating buffer containing recombinant Spike trimer protein at 4° C. Wells were blocked with PBS+1% BSA, and then serum was added to the wells starting at a 1:100 dilution and with serial 1:2 solutions up to 1:12800 in PBS+1% BSA. Samples were incubated at RT for 1 hour, washed 3 times with PBST, and then goat anti-mouse IgG HRP was added at a 1:3000 dilution in PBS+1% BSA and incubated for 1 hour. Plates were again washed 5 times with PBST and developed using the chromogenic HRP substrate 3,3′,5,5′-Tetramethylbenzidine (TMB). The reaction was stopped by addition of H2SO4 and absorbance was measured at 450 nm and 570 nm. Endpoint titer was designated as the highest dilution at which the value of Absorbance at 450-Absorbance at 570 was ≥0.08. At week 3, the endpoint dilution titer in the group immunized with 10 μg of nanoparticle vaccine formulated using PE Dendron_G2-A1-Ricinoleic (FIG. 14) was between 512 and 1024, and continued to rise to an average exceeding 2048 at week 4. This is in contrast to the seronegative group not immunized wherein the titer remained at the baseline of 100 at all time points.

### Example 15. RNA Delivery to Heart and Spleen Tissue

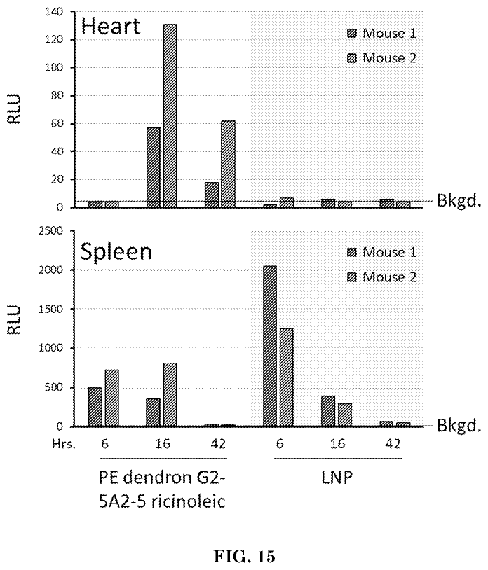

Six BALB/c mice per experimental group were administered the indicated nanoparticle formulations of replicon RNA encoding Luciferase reporter gene by intravenous injection. For the DlinMC₃DMA LNP control formulation, 31.4 μg of RNA was injected, and for the PE dendron G2-5A2-5 ricinoleic formulation, 7.2 μg of RNA was injected. At 6, 16, and 42 hours post-injection two mice were sacrificed and the heart and spleen removed and stored in liquid nitrogen until all timepoints had been collected. To quantify Luciferase gene expression in the isolated hearts and spleens, each whole organ was homogenized in 1 ml of PBS using a VWR® Mini Bead Mill Homogenizer for 30 seconds, and the raw homogenate was centrifuged at 4° C. at 16 000 RCF for 10 minutes. After centrifugation, 150 μL of each supernatant (clarified homogenate) sample was placed in white-walled 96-well assay plates and 150 μL of Pierce™ Firefly Luc One-Step Glow Assay reagent was added. The relative light units (RLU) from each well was measured in a BioTek Synergy HTX microplate reader. The background signal value was defined as the RLU measured for organs of control mice of identical genotype and age that had received no RNA injection. The imaging data show high luciferase expression in heart and spleen for intravenously injected nanoparticles (FIG. 15).

### Example 16. Nanoparticle Compositions Containing Nucleic Acid Carrier of Formula [I]b

Dendritic-linear block-copolymers are hybrids that are typically linear chains that are end-functionalized with dendritic segments. Several groups have reported on benign synthetic approaches for the delivery of hybrid structures that are obtained in excellent yields and contain a bis-MPA dendritic part together (PE) with a linear polymer (P). These hybrids based on PEG have successfully been constructed via CuAAC click chemistries. For these materials, the PEG end-groups containing primary azido intermediate was used for convergent coupling reactions to dendrons, comprising single complementary click groups in the core. The representative structure of these hybrids shown below.

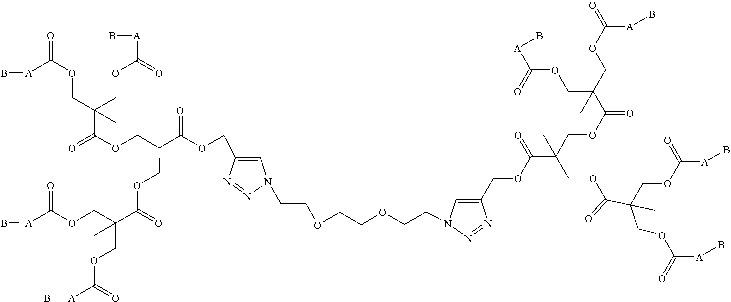

A is an amine linker, B is a hydrophobic unit, and PEG200 is the linker connecting two polyester dendrons.

An example of the synthesis of PE dendron G2 A1 Rici)2 PEG 200 as follows.

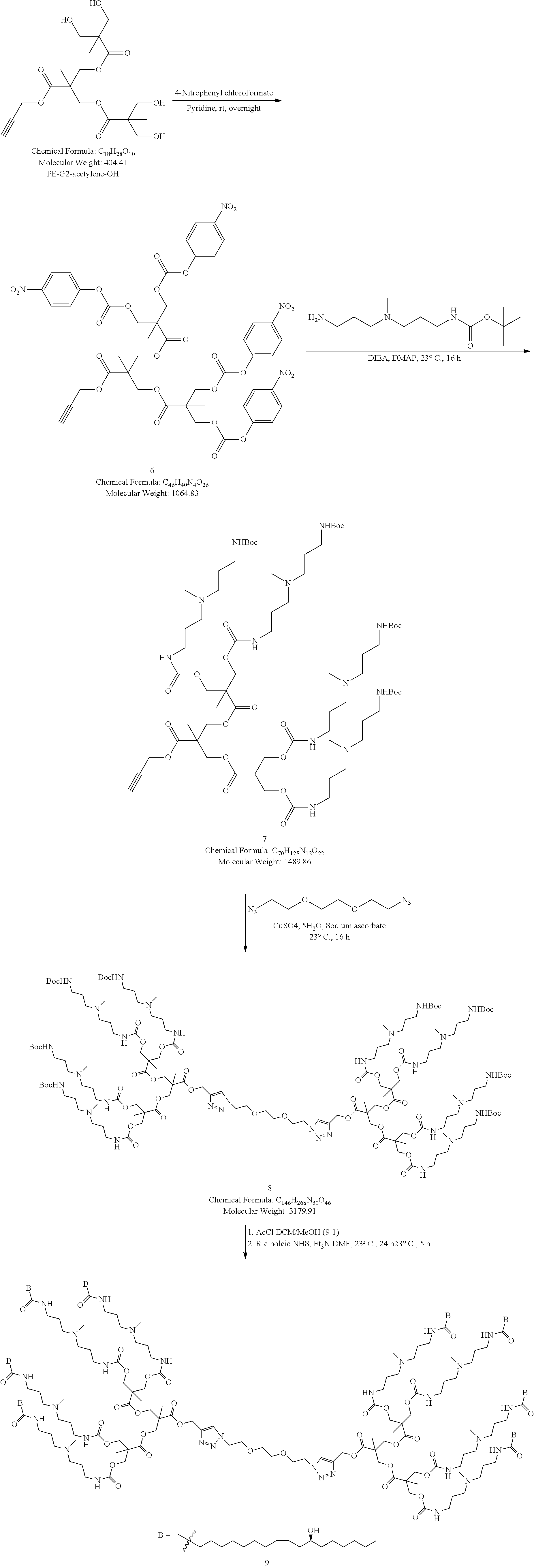

Compound 6: Starting material, PE Dendron G2 acetylene-OH, (300 mg, 0.62 mmol) was dissolved in dry DCM (6 mL) and pyridine (0.9 ml, 4.96 mmol) was added followed by p-nitrophenyl chloroformate (2.1 g, 10 mmol) dissolved in dry DCM (15 mL), the reaction mixture was stirred at 0° C. to 23° C. for 16 h, Next day TLC shows product formation. The reaction mixture was diluted with 1.33 M NaHSO4, extracted with EtOAc. The organic layer was washed with brine and evaporated in Rotavap. The crude was then purified by flash chromatography with DCM/EtOAc. The compound started eluting at 45% EtOAc (R_{f}=0.9 in 1:9 EtOAc/DCM) to yield the desired product as light yellow oil (553 mg, 70%).

Compound 7: A solution of compound 6 (438 mg, 0.41 mmol) dissolved in dry DCM (6 mL) was added to an excess of mono-Boc-DAPMA (0.45 ML, 1.64 mmol) dissolved in dry DCM (6 mL). A solution of DMAP (100 mg, 0.82 mmol) and DIPEA (0.29 ml, 1.64 mmol) in dry DCM (1 mL) was added and the reaction mixture was stirred at 23° C. for 16 h under an argon atmosphere. TLC confirmed the reaction completed. The crude product was concentrated under reduced pressure in Rotavap, and purified via flash chromatography on silica column with gradient elution from 100% CH2Cl2 (mobile phase a) to 75:22:3 CH₂Cl₂/MeOH/NH₄OHaq (by volume, mobile phase b) over 40 minutes. The desired product eluted at 45% mobile phase b. (R_{f}=0.4 in 1:1 mobile phase a/mobile phase b) to yield the desired product as light yellow oil (404 mg, 66%). 1H NMR (301 MHz, CHLOROFORM-d) δ ppm 1.10-1.27 (m, 9H) 1.33-1.43 (m, 36H) 1.52-1.65 (m, 15H) 2.08-2.16 (m, 12H) 2.26-2.37 (m, 16H) 2.51-2.54 (m, 1H) 3.02-3.20 (m, 15H) 3.36-3.42 (m, 3H) 4.01-4.26 (m, 10H) 4.66-4.70 (m, 2H) 5.24-5.27 (m, 3H) 5.28-5.39 (m, 3H) 5.88-5.97 (m, 3H).

Compound 8: PEG-200-Azide (MW: 200, 11.4 mg, 57 μmol) was taken in 50 ml RBF, then compound 7 (MW: 1490, 170 mg, 114 μmol) dissolved in THF (0.6 mL) was added along with CuSO4.5H2O (3 mg, 11.4 μmol, 10 mol %, MW 249.69) and sodium ascorbate (4.5 mg, 22.8 μmol, 20 mol %, MW 198.11), and degassed THF: H₂O (2 mL, 1:1), The reaction mixture was stirred at 23° C. for 16 h. Next day TLC confirmed the reaction completed. The reaction mixture was purified via flash chromatography on silica column with gradient elution from 100% CH2Cl2 (mobile phase a) to 75:22:3 CH₂Cl₂/MeOH/NH₄OHaq (by volume, mobile phase b) over 40 minutes. The desired product eluted at 76% mobile phase b. (R_{f}=0.65 in 75:22:3CH₂Cl₂/MeOH/NH₄OHaq) to yield the desired product as yellow oil (71 mg, 20%). MS (ESI) calcd for C₁₄₆H₂₆₈N₃₀O₄₆ [M+4H]4+m/z 795.5, found 794.9; [M+3H]3+m/z 1059.96, found 1060.2.

Compound 9: 70 mg of compound 8 (0.022 mmol) was treated with 20 eq of AcCl (0.03 ml, 0.44 mmol) after dissolving the compound in 3 ml MeOH, the reaction was stirred at 23° C. for 5 hr, evaporated to dryness and dissolved in 2 ml DMF, added 0.06 ml Et3N (0.44 mmol) followed by 104 mg of Ricinoleic-NHS (synthesized following published protocol: Talukder et al., Publication Number WO/2020/132196, which is incorporated herein by reference as if fully set forth) dissolved in 2 ml DMF. The reaction mixture was stirred at 23° C. for 24 hr, purified via flash chromatography on silica column with gradient elution from 100% CH2Cl2 (mobile phase a) to 75:22:3 CH₂Cl₂/MeOH/NH₄OHaq (by volume, mobile phase b) over 40 minutes. The desired product eluted at 55% mobile phase b. (R_{f}=0.85 (70:24:6 CH₂Cl₂/MeOH/NH₄OHaq) to yield the desired product as yellow oil (60 mg, 43%). 1H NMR (301 MHz, CHLOROFORM-d) δ ppm 0.72-1.00 (m, 28H) 1.03-1.22 (m, 26H) 1.22-1.46 (m, 148H) 1.51-1.59 (m, 13H) 1.91-2.07 (m, 20H) 2.08-2.25 (m, 60H) 2.30-2.46 (m, 33H) 3.11-3.34 (m, 33H) 3.45 (s, 3H) 3.52-3.72 (m, 17H) 3.87 (br s, 4H) 3.98-4.32 (m, 25H) 4.54 (br s, 3H) 5.22 (s, 3H) 5.33-5.58 (m, 16H) 6.12-6.36 (m, 7H) 6.86 (br s, 7H) 7.83 (s, 2H).

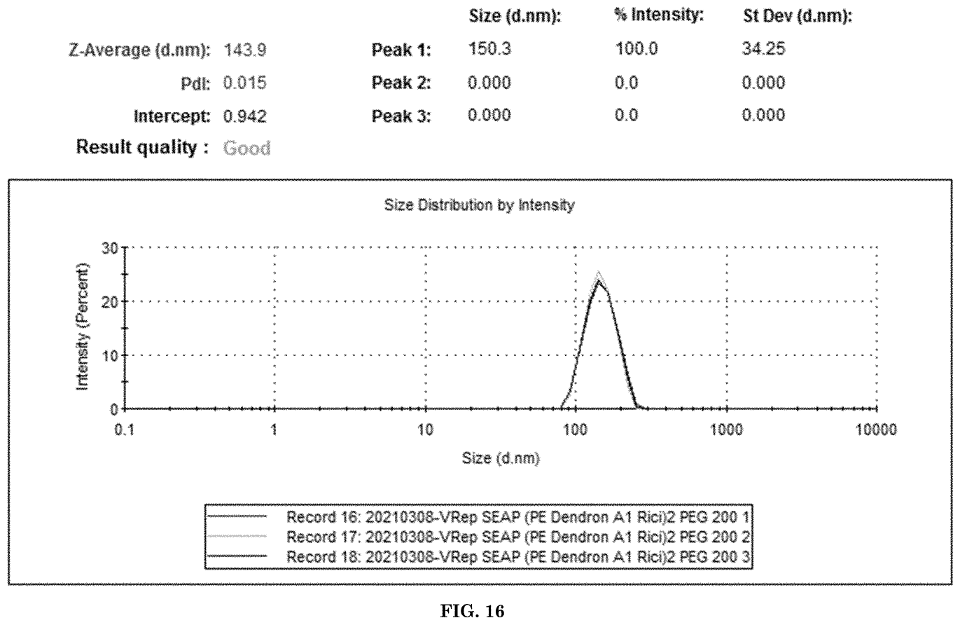

Nanoparticles containing nucleic acid carrier of formula Ib (e.g [PE Dendron_G2-A1-Ricinoleic]2 PEG 200):DSPC:cholesterol:DMG-PEG2k at molar ratios of 1:0.5:2.4:0.035 were formulated using NanoAssemblr Benchtop (Precision NanoSystems Inc, Vancouver, BC, Canada)). RNA was diluted with DNase/RNase-Free, endotoxin free distilled water and sterile citrate buffer to a final desired pH. Total flow rate was maintained at 12 mL per min at a 3:1 ratio of aqueous to organic phase for formulating on the Benchtop. Using glassware washed for 24 hours in 1.0 M NaOH for endotoxin removal and sterilized in a steam autoclave, or depyrogenated by heating at 250° C. for 24 hour, nanoparticles were dialyzed against sterile, endotoxin-free PBS using 20,000 molecular weight cutoff dialysis. Dialyzed nanoparticles were sterile filtered using 0.2 micron poly(ether sulfone) filters and characterized with a Zetasizer NanoZS machine (Malvern). The size distributions were characterized by a single peak with a low polydispersity index, indicating a relatively monodisperse size (FIG. 16).

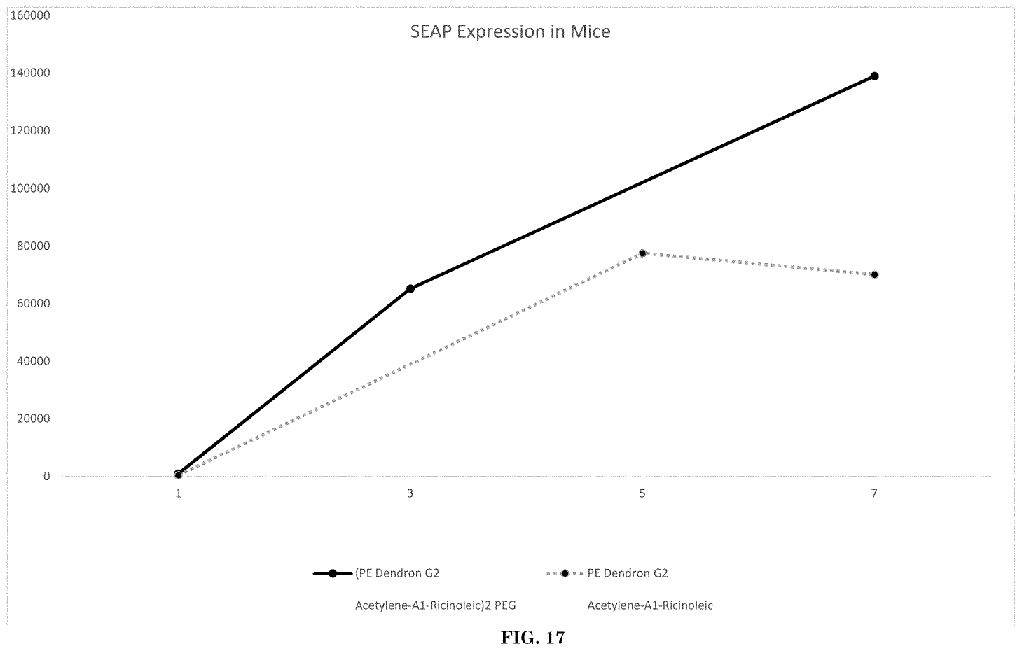

To test formulation of PE dendron G2 A1 Rici)2 PEG 200, the secreted embryonic alkaline phosphatase SEAP reporter system was used. For in vivo tests, mice were injected with nanoparticles at a dose of 2.5 ug of SEAP Replicon RNA, and 1 day, 3 days, 5 days and 7 days later, serum was collected from the mice. The amount of quantified using the Invitrogen NovaBright™ Phospha-Light™ EXP Assay kits for SEAP detection according to the manufacturer's protocol. The amount of SEAP in the mouse serum samples are reported in Arbitrary Units (A.U.) as measured in a BioTek Synergy HTX microplate reader. Error bars are ±S.E.M. Referring to FIG. 17, it was observed that the SEAP amount was higher with the dimer PE dendron G2 A1 Rici)2 PEG 200 than the monomer, PE Dendron_G2-A1-Ricinoleic.

### Example 17. Tracking of the Nanoparticle Compositions

To facilitate tracking of the delivery material in vitro and in vivo, modified dendrimers can have cores containing stable isotopes of carbon (C) or hydrogen (H), such as ¹³C or ²H. When the modified dendrimers are formulated into nanoparticles with nucleic acids, e.g., replicon RNA, they can be tracked in vitro and in vivo post-administration by any known technique, for example, mass spectroscopy or nuclear magnetic resonance imaging. The inclusion of the stable isotopes makes identification of the delivery molecules easier since they become different from the abundant ¹²C and ¹H isotopes that are dominantly found in tissues. Tracking can be useful for identifying biodistribution, material clearance and molecular stability of nanoparticles post-administration, and related issues.

### REFERENCES

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.
(1) (a) Jones, C. H., Chen, C.-K., Ravikrishnan, A., Rane, S., and Pfeifer, B. A. (2013) Overcoming nonviral gene delivery barriers: perspective and future. Mol. Pharmaceutics 10, 4082-4098. (b) Gomes, C. P., Lopes, C. D. F., Moreno, P. M. D., Varela-Moreira, A., Alonso, M. J., and Pêgo, A. P. (2014) Translating chitosan to clinical delivery of nucleic acid-based drugs. MRS Bull. 39, 60-70. (c) Mendes, L. P., Pan, J., Torchilin, V. P. (2017) Dendrimers as Nanocarriers for Nucleic Acid and Drug Delivery in Cancer Therapy. Molecules 22(9), 1401.
(2) Nishikawa, M., and Huang, L. (2001) Nonviral vectors in the new millennium: delivery barriers in gene transfer. Hum. Gene Ther. 12, 861-870
(3) Dufes, C., Uchegbu, I., and Schatzlein, A. (2005) Dendrimers in gene delivery. Adv. Drug Delivery Rev. 57, 2177-2202.
(4) Clare, E. T., Anja, E., and Mark, A. K. (2003) Progress and problems with the use of viral vectors for gene therapy. Nat. Rev. Genet. 4, 346-358.
(5) Mintzer, M. A., and Simanek, E. E. (2009) Nonviral Vectors for Gene Delivery. Chem. Rev. 109, 259-302.
(6) Mintzer, M. A., and Grinstaff, M. W. (2010) Biomedical applications of dendrimers: a tutorial. Chem. Soc. Rev. 40, 173-190.
(7) (a) Raviña, M., de la Fuente, M., Correa, J., Sousa-Herves, A., Pinto, J., Fernandez-Megia, E., Riguera, R., Sanchez, A., and Alonso, M. J. (2010) Core-shell dendriplexes with sterically induced stoichiometry for gene delivery. Macromolecules 43, 6953-6961.
(b) Nouri, A., Castro, R., Kairys, V., Santos, J. L., Rodrigues, J., Li, Y., and Tomás, H. (2012) Insight into the role of N,N-dimethylaminoethyl methacrylate (DMAEMA) conjugation onto poly(ethylenimine): cell viability and gene transfection studies. J. Mater. Sci. Mater. Med. 23, 2967-2980.
(c) Pandita, D., Santos, J. L., Rodrigues, J., Pêgo, A. P., Granja, P. L., and Tomás, H. (2011) Gene delivery into mesenchymal stem cells: a biomimetic approach using RGD nanoclusters based on poly-(amidoamine) dendrimers. Biomacromolecules 12, 472-481.
(d) Rodrigues, J., Jardim, M. G., Figueira, J., Gouveia, M., Tomás, H., and Rissanen, K. (2011) Poly(alkylidenamines) dendrimers as scaffolds for the preparation of low-generation ruthenium based metallodendrimers. New J. Chem. 35, 1938-1943.
(e) Santos, J. L., Oliveira, H., Pandita, D., Rodrigues, J., Pêgo, A. P., Granja, P. L., and Tomás, H. (2010) Functionalization of poly(amidoamine) dendrimers with hydrophobic chains for improved gene delivery in mesenchymal stem cells. J. Controlled Release 144, 55-64.
(f) Santos, J. L., Oramas, E., Pêgo, A. P., Granja, P. L., and Tomás, H. (2009) Osteogenic differentiation of mesenchymal stem cells using PAMAM dendrimers as gene delivery vectors. J. Controlled Release 134, 141-148.
(g) Santos, J. L., Pandita, D., Rodrigues, J., Pêgo, A. P., Granja, P. L., Balian, G., and Tomás, H. (2010) Receptor-mediated gene delivery using PAMAM dendrimers conjugated with peptides recognized by mesenchymal stem cells. Mol. Pharmaceutics 7, 763-774.
(8) (a) Duncan, R., and Izzo, L. (2005) Dendrimer biocompatibility and toxicity. Adv. Drug Delivery Rev. 57, 2215-2237.
(9) (a) Welsh, D. J., Jones, S. P., and Smith, D. K. (2009) “On-off” multivalent recognition: degradable dendrons for temporary high affinity DNA binding. Angew. Chem., Int. Ed. 48, 4047-4051.
(b) Barnard, A., Posocco, P., Pricl, S., Calderon, M., Haag, R., Hwang, M. E., Shum, V. W., Pack, D. W., and Smith, D. K. (2011) Degradable self-assembling dendrons for gene delivery: experimental and theoretical insights into the barriers to cellular uptake. J. Am. Chem. Soc. 133, 20288-20300.
(10) M. A. Carnahan, M. W. Grinstaff, J. Am. Chem. Soc. 123 (2001) 2905. M. A. Carnahan, M. W. Grinstaff, Macromolecules 34 (2001) 7648. M. A. Carnahan, M. W. Grinstaff, Macromolecules 39 (2006) 609.
(11) O. Boussif, F. Lezoualc'h, M. A. Zanta, M. D. Mergny, D. Scherman, B. Demeneix, J. Behr, Proc. Natl. Acad. Sci. U.S.A. 92 (1995) 7297.
(12) LUNDBERG, A., KULKARNI, S., KLEIN, L., PADMANABHAN, H. K. ARATYN, Y. S. COMPOSITIONS AND METHODS FOR GENE EDITING, WO/2018/154387.
(13) Geall A J, Verma A, Otten G R, Shaw C A, Hekele A, Banerjee K, Cu Y, Beard C W, Brito L A, Krucker T, O'Hagan D T, Singh M, Mason P W, Valiante N M, Dormitzer P R, Barnett S W, Rappuoli R, Ulmer J B, Mandl C W. Nonviral delivery of self-amplifying RNA vaccines. Proc Natl Acad Sci USA. 2012 Sep. 4; 109(36):14604-9. doi: 10.1073/pnas.1209367109. Epub 2012 Aug. 20. PMID: 22908294; PMCID: PMC3437863.
(14) World Health Organization. (2002). WHO manual on animal influenza diagnosis and surveillance 2002.5 Rev. 1. 2002.
(15) Poulami Talukder, Jasdave S. Chahal, Justine S. McPartlan, Omar Khan, Karl Ruping. Nanoparticle Compositions for Efficient Nucleic Acid Delivery and Methods of Making and Using the Same. PCT/US19/67402.

It is understood, therefore, that this invention is not limited to the particular embodiments disclosed, but is intended to cover all modifications which are within the spirit and scope of the invention as defined by the appended claims; the above description; and/or shown in the attached drawings.

1. A nucleic acid carrier having a structure of formula Ia or formula Ib: 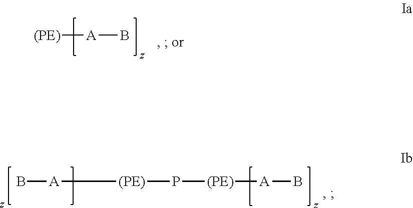
wherein PE is a polyester dendrimer or dendron which includes a core and a plurality of monomeric polyester units that form one or more generations, A is an amine linker, B is a hydrophobic unit, and z is the number of surface groups.

2. The nucleic acid carrier of claim 1, wherein PE has the Formula II: 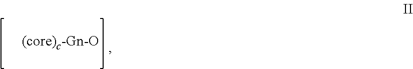
wherein c is the core multiplicity or number of wedges originating from the core, whose values independently range from 1 to 6, G is a layer or generation of dendrimer or dendron and n is a generation number and is in a range from 1 to 10.

3. The nucleic acid carrier of claim 1, wherein the monomeric polyester unit of the plurality is 2,2-bis(hydroxymethyl) propionic acid or 2,2-bis(hydroxymethyl)butyric acid.

4. The nucleic acid carrier of claim 1, wherein z has Formula III:
z=cbⁿ, III
wherein bis branch point multiplicity, or number of branches at each branching point; c is the core multiplicity or number of wedges originating from the core and is in range from 1 to 6, and n is a generation number and is in a range from 1 to 10.

5. The nucleic acid carrier of claim 2, wherein c is 1, and the core is a unidirectional core.

6. The nucleic acid carrier of claim 5, wherein the unidirectional core a carboxylic acid or derivative thereof.

7. The nucleic acid carrier of claim 5, wherein the core is selected from the group consisting of: 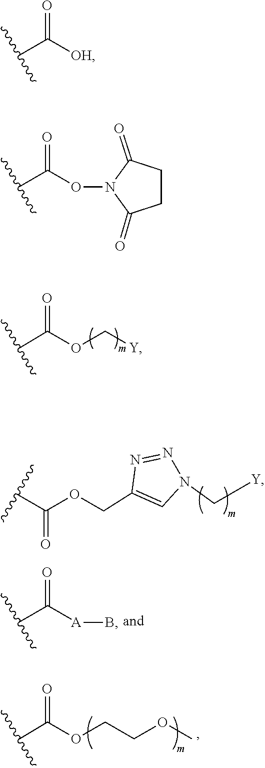
wherein Y is selected from methyl, iso-propyl, sec-butyl, iso-butyl, tert-butyl, isopentyl, neopentyl, 3-methylhexyl, 2,2-dimethylpentyl, 2,3-dimethylpentyl, azide (N3), halogen (Cl, Br, or I), acetylene (C₂H₂), hydroxyl (—OH), or thiol (—SH), -pyranosyl, cycloalkyl, aryl, heteroaryl, and heterocycle; A is an amine linker; B is a hydrophobic unit; and m is 1 to 20.

8. The nucleic acid of claim 7, wherein the cycloalkyl, aryl, heteroaryl, and heterocycle are substituted with at least one group selected from halogen, hydroxyl (—OH) and alkyl group.

9. The nucleic acid carrier of claim 2, wherein c is 3, and the core is a three directional core.

10. The nucleic acid carrier of claim 9, wherein the three directional core is trimethylol propane, or 1,1, 1-tris(hydroxyphenylethane), and has the structure of: 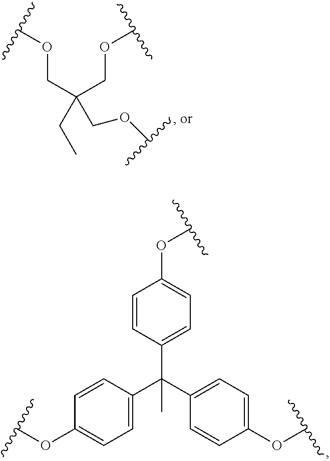 [SMILES: *OCC(CC)(CO*)CO* ; *Oc1ccc(C(C)(c2ccc(O*)cc2)c2ccc(O*)cc2)cc1]
respectively.

11. The nucleic acid carrier of claim 2, wherein cis 4, and the core is a four directional core.

12. The nucleic acid carrier of claim 11, wherein the four directional core is selected from the group consisting of: pentaerythritol, adamantane-1,3,5,7-tetraol, or 5,10,15,20-Tetrakis(4-hydroxyphenyl)-21H,23H-porphine, [1,1′-biphenyl]-3,3′,5,5′-tetraol, 2,3,6,7-tetrahydroxy-9,10-dimethyl-anthracene, 3. 9,10-dimethyl-9,10-dihydro-9,10-ethanoanthracene-2,3,6,7-tetraol, 4. 6,13-dihydro-pentacene-5,7,12, 14-tetraol, Hexahydro-[1,4]dioxino[2,3-b][1,4]dioxine-2,3,6,7-tetraol, Anthracene-1,4,9,10-tetraol, pyrene-1,3,6,8-tetraol, and 3,3,3′,3′-tetramethyl-2,2′,3,3′-tetrahydro-1,1′-spirobi[indene]-5,5′,6,6′-tetrol, and has the structure of: 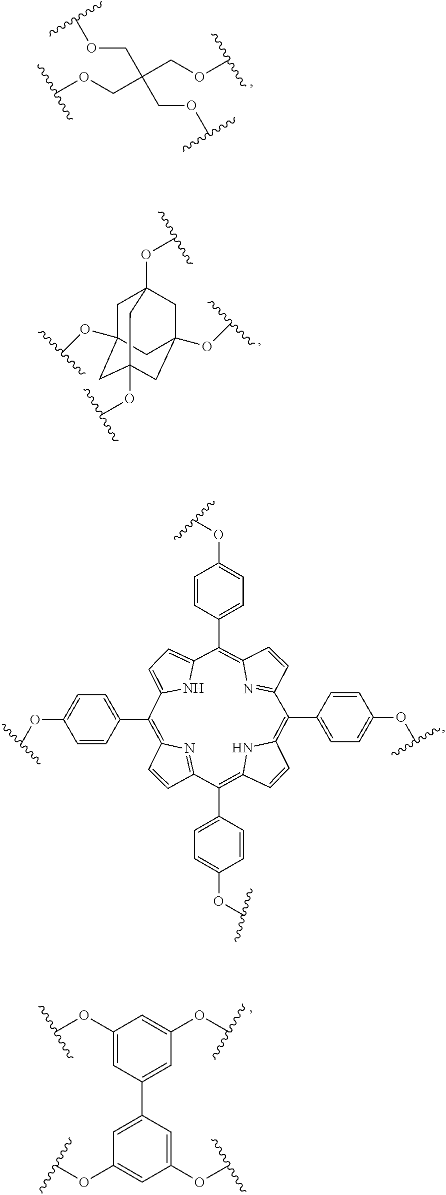 [SMILES: *OCC(CO*)(CO*)CO* ; *OC12CC3(O*)CC(O*)(C1)CC(O*)(C2)C3 ; *Oc1ccc(-c2c3nc(c(-c4ccc(O*)cc4)c4ccc([nH]4)c(-c4ccc(O*)cc4)c4nc(c(-c5ccc(O*)cc5)c5ccc2[nH]5)C=C4)C=C3)cc1 ; *Oc1cc(O*)cc(-c2cc(O*)cc(O*)c2)c1] 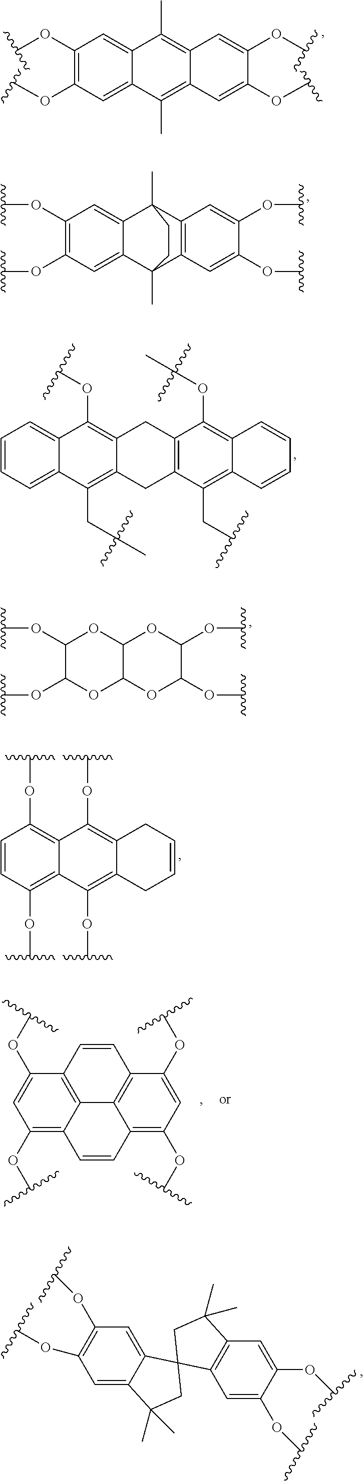
respectively.

13. The nucleic acid carrier of claim 1, wherein A is derived from the group consisting of: N1-(2-aminoethyl)ethane-1,2-diamine. N1-(2-aminoethyl) propane-1,3-diamine, N1-(3-aminopropyl)propane-1,3-diamine, N1,N1′-(ethane-1,2-diyl)bis(ethane-1,2-diamine), N1,N1′-(ethane-1,2-diyl)bis(N2-(2-aminoethyl)ethane-1,2-diamine), N1-(2-(4-(2-aminoethyl)piperazin-1-yl)ethyl)ethane-1,2-diamine, N1-(2-aminoethyl)-N1-methylethane-1,2-diamine, N1-(3-aminopropyl)-N1-methylpropane-1,3-diamine, N1-(3-aminopropyl)-N1-ethylpropane-1,3-diamine, 3-((3-aminopropyl) (methyl)amino)propan-1-ol, 3,3′-(methylazanediyl)bis(propan-1-ol), N1-(3-aminopropyl)-N1-methylbutane-1,4-diamine, 4-((3-aminopropyl)(methyl)amino) butan-1-ol, 4-((3-hydroxypropyl)(methyl)amino)butan-1-ol, 4-((3-hydroxypropyl) (methyl)amino)butan-1-ol, N1-(4-aminobutyl)-N1-methylbutane-1,4-diamine, 4-((4-aminobutyl)(methyl)amino)butan-1-ol, 4,4′-(methylazanediyl)bis(butan-1-ol), 3-((3-aminopropyl)(ethyl)amino)propan-1-ol, 3,3′-(ethylazanediyl)bis(propan-1-ol), N1-(3-aminopropyl)-N1-ethylbutane-1,4-diamine, 4-((3-aminopropyl)(ethyl)amino)butan-1-ol, 4-(ethyl(3-hydroxyprop yl)amino)butan-1-ol, N1-(2-aminoethyl)-N1-methylpropane-1,3-diamine, N1-(4-aminobutyl)-N1-ethylbutane-1,4-diamine, 4,4′-(ethylazanediyl) bis(butan-1-ol), 3-((3-aminopropyl)amino)propan-1-ol, N1-(3-aminopropyl)butane-1,4-diamine, 4-((3-hydroxypropyl)amino)butan-1-ol, N1-(4-aminobutyl)butane-1,4-diamine, 3,3′-azanediylbis(propan-1-ol), 4-((3-aminopropyl)amino)butan-1-ol, 4,4′-azanediylbis(butan-1-ol), and N1,N1′-(butane-1,4-diyl)bis(propane-1,3-diamine); and has the structure of: 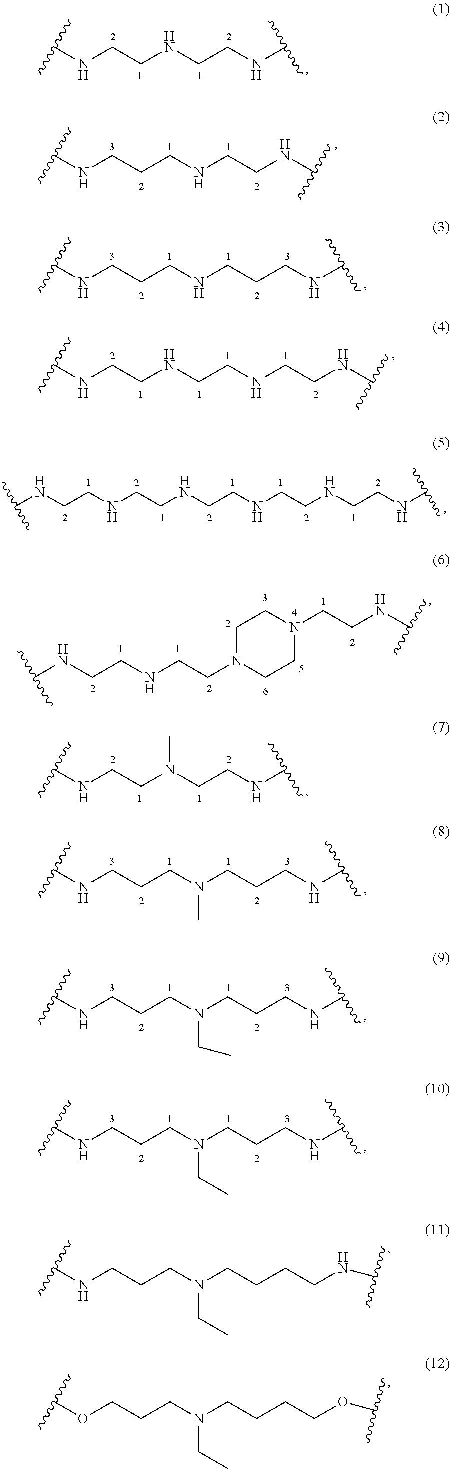 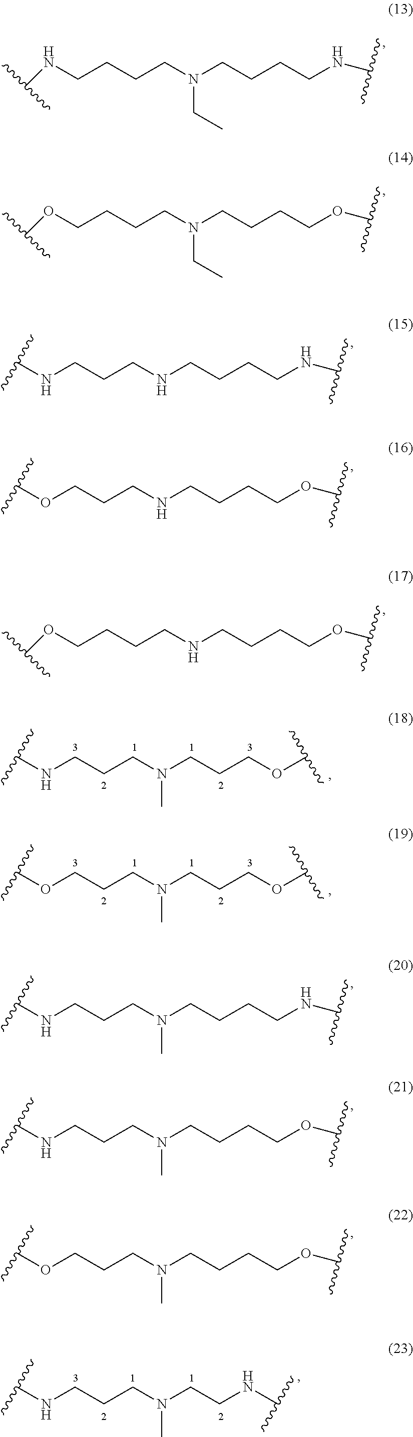 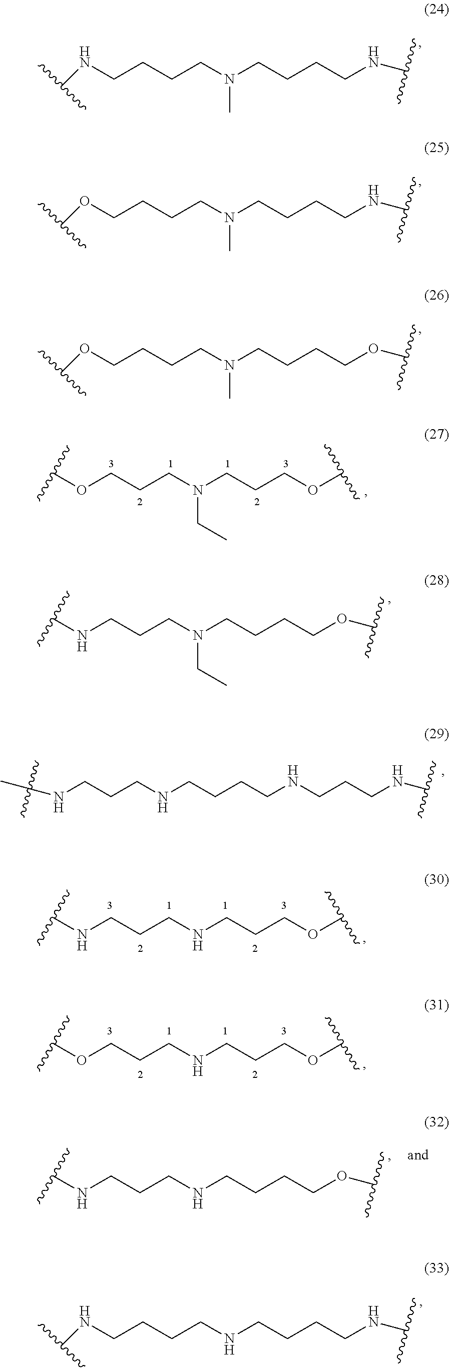
respectively.

14. The nucleic acid carrier of claim 1, wherein B is a C₁-C₂₂ alkyl or C₂-C₂₂ alkenyl group.

15.-17. (canceled)

18. The nucleic acid carrier of claim 1, wherein B is an unsaturated alkyl group.

19.-27. (canceled)

28. The nucleic acid carrier of claim 1, wherein P is homobifunctional linker with two azide groups, and has the structure of Formula IV: 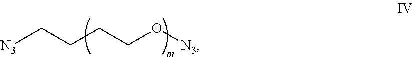
where m is the number ranging from 1 to 20.

29. A nanoparticle composition comprising the nucleic acid carrier of claim 1, and a therapeutic or immunogenic nucleic acid agent enclosed therein.

30. The nanoparticle composition of claim 29, wherein the therapeutic or immunogenic nucleic acid agent is selected from the group consisting of: a polynucleotide, oligonucleotide, DNA, cDNA, RNA, repRNA, siRNA, miRNA, sgRNA, and mRNA.

31.-39. (canceled)

40. A method for treating or preventing a disease or condition in a subject comprising: administering a therapeutically effective amount of the nanoparticle composition of claim 29 to a subject in need thereof.

41. The method of claim 40, wherein the therapeutically effective amount of the nanoparticle composition comprises the therapeutic or immunogenic nucleic acid agent in a range from 0.01 mg nucleic acid to 10 mg nucleic acid per kg body weight of the subject.

42. The method of claim 40, wherein the subject is a mammal.

43. The method of claim 40, wherein the subject is selected from the group consisting of: a chicken, a rodent, a canine, a primate, an equine, a high value agricultural animal, and a human.
